SEQUENCE LISTING (2 sequences)

SEQ ID NO: 1 - protein, 6 aa, Artificial Sequence
  Description of Artificial Sequence Synthetic peptide
KKKEEE

SEQ ID NO: 2 - protein, 6 aa, Artificial Sequence
  Description of Artificial Sequence Synthetic peptide
KKKEEE

(19) United States
(12) Patent Application Publication
(10) Pub. No.: US 2009/0221684 A1
(43) Pub. Date: Sep. 3, 2009

(54) Molecules for Gene Delivery and Gene Therapy, and Methods of Use Thereof

(75) Inventors: Mark W. Grinstaff, Brookline, MA (US); Carla A.H. Prata, Boston, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Trustees of Boston University, Boston, MA (US)

(21) Appl. No.: 12/158,842

(22) PCT Filed: Dec. 20, 2006
(86) PCT No.: PCT/US2006/048693

§ 371 Date: Apr. 21, 2009

Related U.S. Application Data
(60) Provisional application No. 60/752,925, filed on Dec. 22, 2005.

(51) Int. Cl. A61K 48/00; C07F 9/06; C07K 7/06; C12N 15/00; C12N 15/85; C12N 15/82; A61K 31/7052; A61P 43/00

(52) U.S. Cl. 514/44 R; 558/169; 530/329; 435/440; 435/455; 435/468

(57) ABSTRACT

One aspect of the present invention relates to a synthetic non-viral vector composition for gene therapy. Another aspect of the invention relates to the use of the composition for in vitro, ex vivo and/or in vivo transfer of genetic material. The invention also encompasses a pharmaceutical composition (useful for delivery of nucleic acids to a cell), containing a non-cationic amphiphilic molecule or macro-molecule; or a cationic amphiphilic molecule or macromolecule that transforms from a cationic entity to an anionic, neutral, or zwitterionic entity upon a chemical, photochemical, or biological reaction. Another aspect of the invention relates to multicationic compounds that are composed of three or more amino acids. The present invention also relates to the use of the pharmaceutical composition for delivery of nucleic acids to a cell. Moreover, the invention encompasses the non-viral vector compositions tethered to a surface. The surface-tethered compositions are useful for the delivery of nucleic acids to cells in contact with the surface. An additional embodiment of the invention relates to a hydrogel comprising a composition of the invention, and methods of using same for the delivery of genetic material to a cell.

### RELATED APPLICATIONS

This application claims the benefit of the filing date of U.S. Provisional Patent Application Ser. No. 60/752,925, filed Dec. 22, 2005.

### BACKGROUND OF THE INVENTION

In 1972, Friedmann outlined far-reaching opportunities for human gene therapy. Friedmann, T.; Roblin, R. Science 1972, 175, 949-955. Chromosomal deficiencies and/or anomalies, e.g., mutation and aberrant expression, cause many hereditary and non-hereditary diseases. Conventional medicine remains unable to treat many of these diseases; gene therapy may be an effective therapeutic option by either adding, replacing, or removing relevant genes. See Kay, M. A.; Liu, D.; Hoogergrugge, P. M. Proc. Natl. Acad. Sci. 1997, 94, 12744-12746 and Huang, L.; Hung, M.; Wagner, E., Eds. Nonviral Vectors for Gene Therapy; Academic Press: New York, 1999.

Currently few organs or cells can be specifically targeted for gene delivery. There are established protocols for transferring genes into cells, including calcium phosphate precipitation, electroporation, particle bombardment, liposomal delivery, viral-vector delivery, and receptor-mediated gene-delivery. However, a main obstacle to the penetration of a nucleic acid into a cell or target organ lies in the size and polyanionic nature of nucleic acids, both of which militate against their passage across cell membranes. Two strategies currently being explored for delivery of nucleic acids are viral and synthetic non-viral vectors, i.e., cationic molecules and polymers. A brief discussion of viral vectors, cationic lipids, and cationic polymers and their utility in gene therapy is presented below.

### Viral Vectors

Viral vectors are viruses. Viruses, such as adenoviruses, herpes viruses, retroviruses and adeno-associated viruses, are currently under investigation. To date, viral vectors, e.g., adenoviruses and adeno-associated viruses, have exhibited the highest levels of transfection efficiency compared to synthetic vectors, i.e., cationic lipids and polymers. Viral vectors suffer from major disadvantages, such as risks associated with endogenous virus recombination, oncogenic effects, and inflammatory or immunologic reactions. Consequently, the use of viral vectors for human gene therapy is limited. For additional discussion, see Walther, W.; Stein, U. Viral Vectors for Gene Transfer-A Review of their use in the Treatment of Human Diseases Drugs 2000, 60, 249-271; Smith, E. A. Viral Vectors in Gene Therapy Annu. Rev. Microbiol. 1995, 49, 807-838; Drumm, M. L.; Pope, H. A.; Cliff, W. H.; Rommens, J. M.; Marvin, S. A.; Tsui, L. C.; Collins, F. S.; Frizzell, R. A.; Wilson, J. M. Correction of the Cystic-fibrosis Defect in Vitro by Retrovirus-Mediated Gene Transfer Cell 1990, 1990, 1227-1233; Rosenfeld, M. A.; Yoshimura, K.; Trapnell, B. C.; Yoneyama, K.; Rosenthal, E. R.; Dalemans, W.; Fukayama, M.; Bargon, J.; Stier, L. E.; Stratfordperdcaudet, L.; Perricaudet, M.; Guggino, W. B.; Pavirani, A.; Lecocq, J. P.; Crystal, R. G. In vivo Transfer of the Human Cystic-Fibrosis Transmembrane Conductance Regulator Gene to the Airway Epithelium Cell 1992, 68, 143-155; Muzyczka, N. Use of Adenoassociated Virus as a General Transduction Vector for Mammalian Cells Curr. Top. Microbiol. Immuno. 1992, 158, 97-129; Robbins, P. D.; Tahara, H.; Ghivizzani, S. C. Viral Vectors for Gene Therapy Trends Biotechnol 1998, 16, 35-40; and oss, G.; Erickson, R.; Knorr, D.; Motulsky, A. G.; Parkman, R.; Samulski, J.; Straus, S. E.; Smith, B. R. Gene Therapy in the United States: A Five-Year Status Report Hum. Gene Ther. 1996, 14, 1781-1790.

In particular, because this method infects an individual cell with a viral carrier, a potentially life threatening immune response to the treatment can develop. Summerford reviews gene therapy with Adeno-associated viral vectors. For additional details, see: Marshall, E. Clinical Research—FDA Halts All Gene Therapy Trials at Penn Science 2000, 287, 565-567; and Summerford, C.; Samulski, R. J. Adeno-associated Viral Vectors for Gene Therapy Biogenic Amines 1998, 14, 451-475. Several examples of viral vectors used for gene delivery are described below. In U.S. Pat. No. 5,585,362, Wilson et al. described an improved adenovirus vector and methods for making and using such vectors. Likewise, U.S. Pat. No. 6,268,213 to Samulski et al. describes an adeno-associated virus vector and cis-acting regulatory and promoter elements capable of expressing at least one gene and method of using the viral vector for gene therapy. Although the transfection efficiency is high with viral vectors, there are a number of complications associated with the use of viral vectors.

### Cationic Lipids

The second strategy consists of using non-viral agents capable of promoting the transfer and expression of DNA in cells. Since the first report by Felgner, this area has been actively investigated. These cationic non-viral agents bind to polyanionic DNA. Following endocytosis, the nucleic acid must escape from the delivery agent as well as the endosomal compartment so that the genetic material is incorporated within the new host. The mechanism of nucleic acid transfer from endosomes to cytoplasm and/or nuclear targets is still unclear. Possible mechanisms are simple diffusion, transient membrane destabilization, or simple leakage during a fusion event in which endosomes fuse with other vesicles. See Felgner, P. L. Nonviral Strategies for Gene Therapy Sci. Am. 1997, 276, 102-106; Felgner, P. L.; Gadek, T. R.; Holm, M.; Roman, R.; Chan, H. W.; Wenz, M.; Northrop, J. P.; Ringgold, G. M.; Danielsen, M. Lipofectin: A highly efficient, lipid mediated DNA-transfection procedure Proc. Natl. Acad. Sci. USA 1987, 84, 7413-7417; Felgner, P. L.; Kumar, R.; Basava, C.; Border, R. C.; Hwang-Felgner, J. In; Vical, Inc. San Diego, Calif.: U.S. Pat. No. 5,264,618; Felgner, J. H.; Kumar, R.; Sridhar, C. N.; Wheeler, C. J.; Tsai, Y. J.; Border, R.; Ramsey, P.; Martin, M.; Felgner, P. L. Enhanced Gene Delivery and Mechanism Studies with a Novel Series of Cationic Formulations J. Biol. Chem. 1994, 269, 2550-2561; Freidmann, T. Sci. Am. 1997, 276, 96-101; Behr, J. P. Gene Transfer with Synthetic Cationic Amphiphiles: Prospects for Gene Delivery Bioconjugate Chem. 1994, 5, 382-389; Cotton, M.; Wagner, E. Non-viral Approaches to Gene Therapy Curr. Op. Biotech. 1993, 4, 705-710; Miller, A. D. Cationic Liposomes for Gene Therapy Angew. Chem. Int. 1998, 37, 1768-1785; Scherman, D.; Bessodes, M.; Cameron, B.; Herscovici, J.; Hofland, H.; Pitard, B.; Soubrier, F.; Wils, P.; Crouzet, J. Application of Lipids and Plasmid Design for Gene Delivery to Mammalian Cells Curr. Op. Biotech. 1989, 9, 480; Lasic, D. D. In Surfactants in Cosmetics; 2nd ed.; Rieger, M. M., Rhein, L. D., Eds.; Marcel Dekker, Inc.: New York, 1997; Vol. 68, pp 263-283; Rolland, A. P. From Genes to Gene Medicines: Recent Advances in Nonviral Gene Delivery Crit. Rev. Ther. Drug 1998, 15, 143-198; de Lima, M. C. P.; Simoes, S.; Pires, P.; Faneca, H.; Duzgunes, N. Cationic Lipid-DNA Complexes in Gene Delivery from Biophysics to Biological Applications Adv. Drug. Del. Rev. 2001, 47, 277-294.

These synthetic vectors have two main functions: to condense the DNA to be transfected; and to promote its cell-binding and passage across the plasma membrane, and where appropriate, the two nuclear membranes. Due to its polyanionic nature, DNA naturally has poor affinity for the plasma membrane of cells, which is also polyanionic. Several groups have reported the use of amphiphilic cationic lipid-nucleic acid complexes for in vivo transfection both in animals and humans. Thus, non-viral vectors have cationic or polycationic charges. See Gao, X.; Huang, L. Cationic Liposome-mediated Gene Transfer Gene Therapy 1995, 2, 710-722; Zhu, N.; Liggott, D.; Liu, Y.; Debs, R. Systemic Gene Expression After Intravenous DNA Delivery into Adult Mice Science 1993, 261, 209-211; and Thierry, A. R.; Lunardiiskandar, Y.; Bryant, J. L.; Rabinovich, P.; Gallo, R. C.; Mahan, L. C. Systemic Gene-Therapy-Biodistribution and Long-Term Expression of a Transgene in Mice Proc. Nat. Acad. Sci. 1995, 92, 9742-9746.

Cationic amphiphilic compounds that possess both cationic and hydrophobic domains have been used previously for delivery of genetic information. In fact, this class of compounds is widely used for intracellular delivery of genes. Such cationic compounds can form cationic liposomes which are the most popular synthetic vector system for gene transfection studies. The cationic liposomes serve two functions. First, they protect the DNA from degradation. Second, they increase the amount of DNA entering the cell. While the mechanisms describing how cationic liposomes function have not been fully delineated, such liposomes have proven useful in both in vitro and in vivo studies. Safinya, C. R. describes the structure of the cationic amphiphile-DNA complex. See Radler, J. O.; Koltover, I.; Salditt, T.; Safinya, C. R. Science 1997, 275, 810-814; Templeton, N. S.; Lasic, D. D.; Frederik, P. M.; Strey, H. H.; Roberts, D. D.; Pavlakis, G. N. Nature Biotech. 1997, 15, 647-652; Koltover, I.; Salditt, T.; Radler, J. O.; Safinya, C. R. Science 1998, 281, 78-81; and Koltover, I.; Salditt, T.; Safinya, C. R. Biophys. J. 1999, 77, 915-924. Many of these systems for gene delivery in vitro and in vivo are reviewed in recent articles. See Remy, J.; Sirlin, C.; Vierling, P.; Behr, J. Bioconj. Chem. 1994, 5, 647-654; Crystal, R. G. Science 1995, 270, 404-410; Blaese, X.; et, a. Cancer Gene Ther. 1995, 2, 291-297; and Behr, J. P. and Gao, X cited above. Unlike viral vectors, the lipid-nucleic acid complexes can be used to transfer expression cassettes of essentially unlimited size.

Because these synthetic delivery systems lack proteins, they may evoke fewer immunogenic and inflammatory responses. However, the liposomes suffer from low transfection efficiencies. Moreover, as is the case with other polycations, cationic lipids and liposomes (e.g., Lipofectin®) can be toxic to the cells and inefficient in their DNA delivery in the presence of serum. See Leonetti et al. Behr, like Leonetti, reports that these cationic amphiphiles or lipids are adversely affected by serum and some are toxic. See Leonetti, J.; Machy, P.; Degols, G.; Lebleu, B.; Leserman, L. Proc. Nat. Acad. Sci. 1990, 87, 2448-2451 and Behr, J. P. Acc. Chem. Res. 1993, 26, 274-278.

Behr discloses amphiphiles including dioctadecylamidologlycylspermine (“DOGS”) for gene delivery. This material is commercially available as TRANSFECTAM®. Vigneron describes guanidinium-cholesterol cationic lipids for transfection of eukaryotic cells. Felgner discloses use of positively-charged synthetic cationic lipids including N-1-(2,3-dioleyloxy)propyl-N,N,N-trimethylammonium chloride (“DOTMA”), to form lipid/DNA complexes suitable for transfections. Byk describes cationic lipids where the cationic portion of the amphiphile is either linear, branched, or globular for gene transfection. Blessing and coworkers describe a cationic synthetic vector based on spermine. Safinya describes cationic lipids containing a poly(ethylene glycol) segment for gene delivery. Bessodes and coworkers describe a cationic lipid containing glycosidic linker for gene delivery. Ren and Liu describe cationic lipids based on 1,2,4-butanetriol. Tang and Scherman describe a cationic lipid that contains a disulfide linkage for gene delivery. Vierling describes highly fluorinated cationic amphiphiles as gene carrier and delivery systems. Jacopin describes a cation amphiphile for gene delivery that contains a targeting ligand. Wang and coworkers describe carnitine based cationic esters for gene delivery. Zhu describes the use of a cationic lipid, N[1-(2,3-dioleyloxy)propyl]-N,N,N-trimethylammonium chloride, for the intravenous delivery of DNA. See Behr, J. P.; Demeneix, B.; Loeffler, J. P.; Perez-Mutul, J. Efficient Gene Transfer into Mammalian Primary Endocrine Cells with Lipopolyamine Coated DNA Proc. Nat. Acad. Sci. 1989, 86, 6982-6986; Vigneron, J. P.; Oudrhiri, N.; Fauquet, M.; Vergely, L.; Bradley, J. C.; Basseville, M.; Lehn, P.; Lehn, J. M. Proc. Nat. Acad. Sci. 1996, 93, 9682-9686; Byk, G.; BDubertret, C.; Escriou, V.; Frederic, M.; Jaslin, G.; Rangara, R.; Pitard, B.; Wils, P.; Schwartz, B.; Scherman, D. J. Med. Chem. 1998, 41, 224-235; Blessing, T.; Remy, J. S.; Behr, J. P. J. Am. Chem. Soc. 1998, 120, 8519-8520; Blessing, T.; Remy, J. S.; Behr, J. P. Proc. Nat. Acad. Sci. 1998, 95, 1427-1431; Schulze, U.; Schmidt, H.; Safinya, C. R. Bioconj. Chem. 1999, 10, 548-552; Bessodes, M.; Dubertret, C.; Jaslin, G.; Schennan, D. Bioorg. Med. Chem. Lett. 2000, 10, 1393-1395; Herscovici, J.; Egron, M. J.; Quenot, A.; Leclercq, F.; Leforestier, N.; Mignet, N.; Wetzer, B.; Scherman, D. Org. Lett. 2001; Ren, T.; Liu, D. Tetrahedron Lett. 1999, 40, 7621-7625; Tang, F.; Hughes, J. A. Biochem. Biophys. Res. Commun. 1998, 242, 141-145; Tang, F.; Hughes, J. A. Bioconjugate Chem. 1999, 10, 791-796; Wetzer, B.; Byk, G.; Frederic, M.; Airiau, M.; Blanche, F.; Pitard, B.; Scherman, D. Biochemical J. 2001, 356, 747-756; Vierling, P.; Santaella, C.; Greiner, J. J. Fluorine Chem. 2001, 107, 337-354; Jacopin, J.; Hofland, H.; Scherman, D.; Herscovici, J. J. Biomed. Chem. Lett. 2001, 11, 419-422; and Wang, J.; Guo, X.; Xu, Y.; Barron, L.; Szoka, F. C. J. Med. Chem. 1998, 41, 2207-2215.

U.S. Pat. No. 5,283,185 to Epand et al. describes additional examples of amphiphiles, including a cationic cholesterol synthetic vector, termed “DC-chol”. U.S. Pat. No. 5,264,6184 describes more cationic compounds that facilitate transport of biologically active molecules into cells. U.S. Pat. Nos. 6,169,078 and 6,153,434 to Hughes et al. disclose a cationic lipid that contains a disulfide bond for gene delivery. U.S. Pat. No. 5,334,761 to Gebeyehu et al. describes additional cationic amphiphiles suitable for intracellular delivery of biologically active molecules. U.S. Pat. No. 6,110,490 to Thierry describes additional cationic lipids for gene delivery. U.S. Pat. No. 6,056,938 to Unger, et al. discloses cationic lipid compounds that contain at least two cationic groups.

### Cationic Polymers

Recently, polymeric systems have been explored for gene delivery. In Han's review, he discussed most of the common cationic polymer systems including PLL, poly(L-lysine); PEI, polyethyleneimine; pDMEAMA, poly(2-dimethylamino)ethyl-methacrylate; PLGA, poly(D,L-lactide-co-glycolide) and PVP (polyvinylpyrrolidone). See Garnett, M. C. Crit. Rev. Ther. Drug Carrier Sys. 1999, 16, 147-207; Han, S.; Mahato, R. I.; Sung, Y. K.; Kim, S. W. Molecular Therapy 2000, 2, 302-317; Zauner, W.; Ogris, M.; Wagner, E. Adv. Drug. Del. Rev. 1998, 30, 97-113; Kabanov, A. V.; Kabanov, V. A. Bioconj. Chem. 1995, 6, 7-20; Lynn, D. M.; Anderson, D. G.; Putman, D.; Langer, R. J. Am. Chem. Soc. 2001, 123, 8155-8156; Boussif, O.; Lezoualc'h, F.; Zanta, M. A.; Mergny, M. D.; Scherman, D.;. Demeneix, B.; Behr, J. P. Proc. Natl. Acad. Sci. USA 1995, 92, 7297-7301; Choi, J. S.; Joo, D. K.; Kim, C. H.; Kim, K.; Park, J. S. J. Am. Chem. Soc. 2000, 122, 474-480; Putnam, D.; Langer, R. Macromolecules 1999, 32, 3658-3662; Gonzalez, M. F.; Ruseckaite, R. A.; Cuadrado, T. R. Journal of Applied Polymer Science 1999, 71, 1223-1230; Tang, M. X.; Redemann, C. T.; Szoka, F. C. In Vitro Gene Delivery by Degraded Polyamidoamine Dendrimers Bioconjugate Chem. 1996, 7, 703-714; Kukowska-latallo, J. F.; Bielinska, A. U.; Johnson, J.; Spinder, R.; Tomalia, D. A.; Baker, J. R. Proc. Nat. Acad. Sci. 1996, 93, 4897-4902; and Lim, Y.; Kim, S.; Lee, Y.; Lee, W.; Yang, T.; Lee, M.; Suh, M.; Park, J. J. Am. Chem. Soc. 2001, 123, 2460-2461.

Some representative examples of cationic polymers under investigation are described below. For example, poly(β-amino esters) have been explored and shown to condense plasmid DNA into soluble DNA/polymer particles for gene delivery. To accelerate the discovery of synthetic transfection vectors parallel synthesis and screening of a cationic polymer library was reported by Langer. Wolfert describes cationic vectors for gene therapy formed by self-assembly of DNA with synthetic block cationic co-polymers. Haensler and Szoka describe the use of cationic dendrimer polymers polyamidoamine (PAMAM) dendrimers) for gene delivery. Wang describes a cationic polyphosphoester for gene delivery. Putnam describes a cationic polymer containing imidazole for the delivery of DNA. See Lynn, D. M.; Langer, R. J. Am. Chem. Soc. 2000, 122, 10761-10768; Wolfert, M. A.; Schacht, E. H.; Toncheva, V.; Ulbrich, K.; Nazarova, O.; Seymour, L. W. Hum. Gene Ther. 1996, 7, 2123-2133; Haensler, J.; Szoka, F. Bioconj. Chem. 1993, 4, 372; and Wang, J.; Mao, H. Q.; Leong, K. W. J. Am. Chem. Soc. 2001; Putnam, D.; Gentry, C. A.; Pack, D. W.; Langer, R. Proc. Nat: Acad. Sci. 2001, 98, 1200-1205.

A number of patents describe cationic polymers for gene delivery. For example, U.S. Pat. No. 5,629,184 to Goldenberg et al. describes cationic copolymers of vinylamine and vinyl alcohol for the delivery of oligonucleotides. U.S. Pat. No. 5,714,166 to Tomalia et al. discloses dendritic cationic-amine-terminated polymers for gene delivery. U.S. Pat. No. 5,919,442 to Yin et al. describes cationic hyper comb-branched polymer conjugates for gene delivery. U.S. Pat. No. 5,948,878 to Burgess et al. describes additional cationic polymers for nucleic acid transfection and bioactive agent delivery. U.S. Pat. No. 6,177,274 to Park et al. discloses a compound for targeted gene delivery that consists of polyethylene glycol (PEG) grafted poly(L-lysine) (PLL) and a targeting moiety, wherein at least one free amino function of the PLL is substituted with the targeting moiety, and the grafted PLL contains at least 50% unsubstituted free amino function groups. U.S. Pat. No. 6,210,717 to Choi et al. describes a biodegradable, mixed polymeric micelle used to deliver a selected nucleic acid into a targeted host cell that contains an amphiphilic polyester-polycation copolymer and an amphiphilic polyester-sugar copolymer. U.S. Pat. No. 6,267,987 to Park et al. discloses a positively charged poly[alpha-(omega-aminoalkyl) glycolic acid] for the delivery of a bioactive agent via tissue and cellular uptake. U.S. Pat. No. 6,200,956 to Scherman et al. describes a pharmaceutical composition useful for transfecting a nucleic acid containing a cationic polypeptide.

All of these polymers possess and rely on cationic moieties to bind DNA. Thus, non-cationic polymers or macromolecules and polymers or macromolecules that change their overall charge (i.e., charge-reversing or charge-switching) for gene delivery have not been described. Such polymers would also be advantageous over using viral vectors because the polymer delivery system would not expose the cell to a virus that could infect the cell.

Therefore, the need exists for new compositions and methods for gene delivery. New gene delivery compositions will find applications in medicine and gene research. Remarkably, the present invention fulfills this need and others, and provides additional related advantages.

### SUMMARY OF THE INVENTION

This present invention relates to the field of compounds and methods for gene delivery. One aspect of the invention relates to a class of cationic amphiphilic molecules or macromolecules useful for gene delivery that transform into an anionic, neutral, or zwitterionic entity by a chemical, photochemical, or biological reaction. Another aspect of the invention relates to zwitterionic amphiphilic molecules or macromolecules that transform into an anionic or neutral entity by a chemical, photochemical, or biological reaction. Another aspect of the invention relates to a method of delivering a gene or oligonucleic acid to a cell using a molecule of the invention that changes charge to an anionic, neutral, or zwitterionic state through a chemical, photochemical, or biological reaction. Another aspect of the invention relates to a method of delivering a gene or oligonucleic acid to a cell using a zwitterionic compound in combination with a cationic lipid, such as DOTAP. Another aspect of the invention relates to a method of delivering a gene or nucleic acid to a cell using said charge-reversing cationic amphiphiles and a cationic amphiphile (non-charge reversing amphiphile). Another aspect of the invention relates to multicationic compounds that are composed of three or more amino acids. In another embodiment, the invention relates to a hydrogel comprising a compound of the present invention. In another embodiment, the present invention relates to use of such a hydrogel for the delivery of genetic material to a cell. The delivery of said compositions and nucleic acids can be in vitro, ex vivo or in vivo.

### BRIEF DESCRIPTION OF THE FIGURES

FIG. 1 illustrates the charge-reversing transformation of an amphiphilic molecule from a net cation to a net anion. Due to its overall charge the net cationic amphiphile binds DNA, and then releases DNA when it is transformed to the net anionic compound.

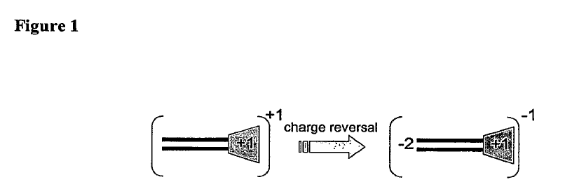

FIG. 2 depicts schematically structural regions, and various combinations thereof, that may be comprised by a molecule or macromolecule of the invention.

FIG. 3 depicts schematically structural regions, and various combinations thereof, that may be comprised by a molecule or macromolecule of the invention.

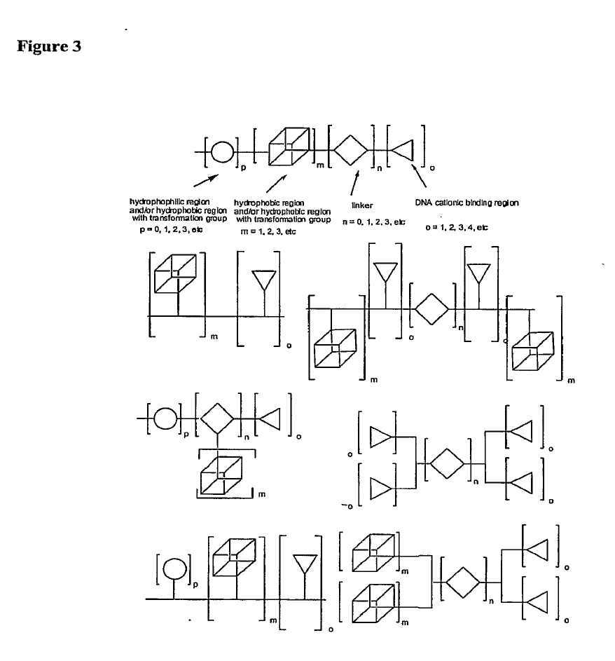

FIG. 4 depicts certain molecules or macromolecules of the invention.

FIG. 5 depicts certain molecules or macromolecules of the invention.

FIG. 6 depicts certain molecules or macromolecules of the invention.

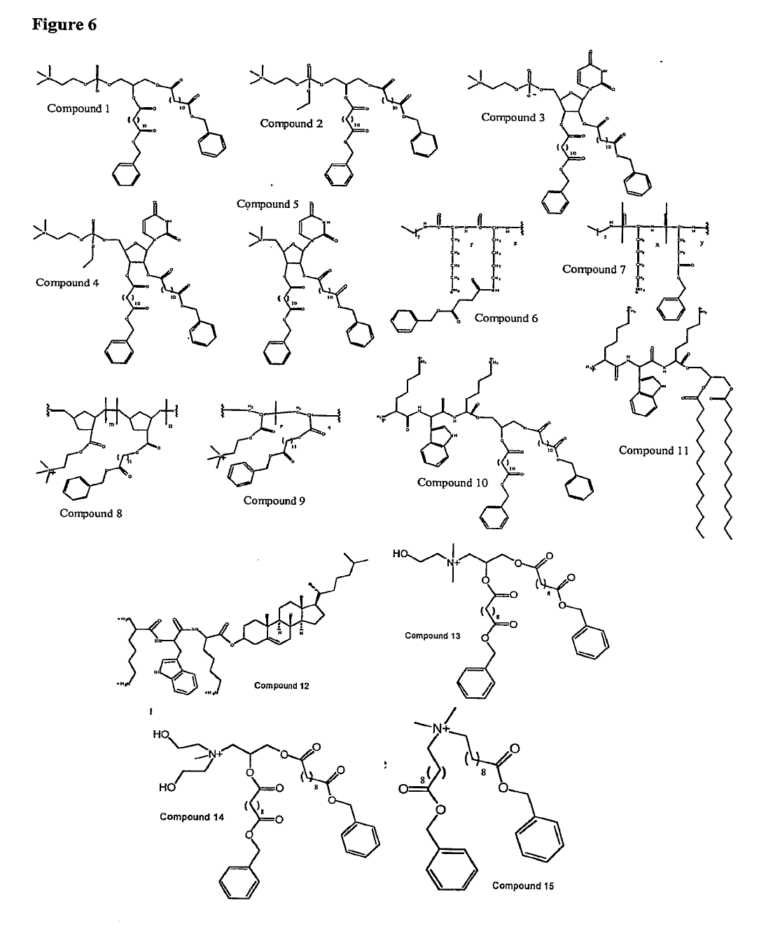

### DETAILED DESCRIPTION OF THE INVENTION

One aspect of the present invention relates to molecules and macromolecules and compositions of either of them, which are useful for in vitro, ex vivo, and in vivo transfer of biologically active molecules, such as endogenous and exogenous genes and oligonucleic acids. The present invention also encompasses methods of using said molecules and macromolecules and compositions of either of them for gene deliver or gene therapy in vitro, ex vivo or in vivo (e.g., in a mammal, bovine, canine, feline, equinine, porcine, rodent, primate, or human). In certain embodiments, the molecule or macromolecule contains at least one nucleic acid binding region (which is cationic), a linker, and at least one hydrophobic region. Alternatively, the molecule or macromolecule is a cationic amphiphilic molecule or macromolecule that transforms from a net cationic entity to a net anionic, neutral, or zwitterionic entity by a chemical, photochemical, or biological reaction. The present invention also relates to a method of using such a molecule or macromolecule for in vitro, ex vivo or in vivo delivery of an endogenous or exogenous gene or oligonucleic acid.

Yet another embodiment of the invention relates to such molecules or macromolecules tethered to a surface. In certain embodiments, the present invention relates to a method of delivering a gene or oligonucleotide to a cell, comprising contacting a cell with a surface comprising a tethered molecule or macromolecule of the present invention, wherein said molecule or macromolecule comprises a gene or oligonucleic acid. An additional embodiment of the invention relates to a hydrogel, comprising a plurality of molecules or macromolecules of the present invention; and a gene or oligonucleic acid. The present invention also relates to a method of gene delivery or gene therapy, comprising contacting a cell with an aforementioned hydrogel.

Another aspect of the invention relates to multicationic compounds that are composed of three or more amino acids. In certain embodiments, said compounds contain three or more amino acids and two or more lipid or hydrophobic chains, and have an overall positive charge.

In certain embodiments, the invention relates to a method of delivering a gene or oligonucleic acid to a cell, comprising the step of subjecting to a change in the ionic strength of the surrounding environment a molecule or macromolecule of the invention comprising a gene or an oligonucleotide. For example, the change in ionic strength may occur when the molecule or macromolecule of the invention comprising a gene or oligonucleic acid enters the cytoplasm of a cell, thereby resulting in partial or complete release of the gene or oligonucleotide into the cytoplasm.

In general, the molecules and macromolecules of the invention are chemical-, photochemical-, or biochemical-sensitive cationic amphiphile molecules or polymer/macromolecules for gene delivery that transform to an anionic or neutral amphiphile or polymer, e.g., intracellularly. In certain embodiments, the functional synthetic vectors of the invention perform the following roles. First, it binds DNA and forms a supermolecular DNA-complex. Then, the DNA-complex penetrates the cell membrane. Once this complex is inside the cell, one or more chemical, photochemical, or biochemical reactions affords a synthetic vector that is anionic or neutral. Finally, the charge-changed amphiphiles or polymers electostatically release or liberate or expel the complexed DNA, by virtue of the destabilized supramolecular complex, and the released DNA is available for subsequent transcription. For example, a cationic amphiphile possessing one to two terminal ethyl or benzyl ester linkages on the fatty acid is an esterase sensitive functional synthetic vector. This cationic amphiphile would bind DNA and form the supramolecular complex. An esterase would then cleave the ester linkages affording the anionic amphiphile and freeing the DNA. Another example, would be a cationic amphiphile possessing one or two ester linkages that can be cleaved by a photochemical reaction. Photocleavable protecting groups for use in this invention include nitrobenzyl, 6-bromo-7-hydroxy-coumarin-4-ylmethyl (bhc), 8-bromo-7-hydroxyquinoline-2-ylmethyl (bhq), 4-methoxy-5,7-dinitroimdoliyl (MDNI), and 4-methoxy-7-nitroimdolinyl (MNI). The release of the DNA from the amphiphile-DNA complex in vitro or in vivo is done by photolysis (one or more photon chemistry).

The composition comprising a nucleic acid and a molecule or macromolecule of the present invention may take the form of a liquid, gel, or solid, depending on the environment, and the presence or absence of solvent. A nucleoside possessing two fatty acid chains and a phosphocholine will often form a gel in aqueous solution. Such an example is synthesized and described in the examples section. Moreover, this gel can be loaded with DNA or DNA and a synthetic vector and subsequently used to deliver nucleic acid to a specific tissue/cellular site. This mode of gene therapy is applicable to cancer.

Nucleic acids suitable for delivery include, but are not limited to, DNA, RNA, plasmids, siRNA, duplex oligonucleotides, single-stranded oligonucleotides, triplex oligonucleotides, PNAs, mrRNA, and the like. Delivery of nucleic acid using the novel molecule(s) or polymer(s) described in this invention may be in vitro, ex vivo, and in vivo (e.g., intravenous, intramuscular, aerosol, oral, topical, systemic, ocular, intraperitoneal and/or intrathecal). The administration can also be directed to a target tissue/cell or through systemic delivery. The synthetic vectors described here can be further modified to possess unique peptides, antibodies, single-chain antibodies, or other small molecules that target the delivery of the DNA to a specific cell.

A further embodiment of the invention is the use of the functional synthetic vectors (i.e., the molecules and macromolecules of the present invention) with known, standard, or conventional synthetic vectors (molecules and polymers) and/or cationic (e.g., DOTAP, DOTMA, Transfast, Lipofecamine), anionic, zwitterionic lipids or amphiphiles (e.g., DOPE) for the delivery of DNA. Moreover, the synthetic vectors described herein can be used with known peptides or polymers that lyse or destabilize cell membranes, thereby increasing the release of the DNA from the endosome (e.g., polyacrylic acids/alkyl-esters). The synthetic vectors may be used in combination with amphiphilic polymers, macromolecules, peptides, and/or antibodies that, e.g., direct the nucleic acid to the nucleus.

With respect to the amphiphilic molecules or macromolecules, the present invention also relates to a liposome comprising one or more of them, and related compositions and methods of preparing said liposomes. Moreover, the present invention relates to methods of administering to a cell the aforementioned biologically-active-agent/liposome compositions. The modified cells may be used in an in vitro setting or delivered to a patient. Alternatively, the therapeutic liposome formulation is delivered to a patient, resulting in in vivo modification of a patient's cells. In other words, the aforementioned liposomal compositions of the present invention may be used in a method for delivery of nucleic acids into cells. The liposome vesicles may be prepared from a mixture comprising a nucleic acid, one or more amphiphile(s) of the present invention, and a neutral lipid, which forms a bi- or multi-lamellar membrane structure. In other words, the present invention also relates to a method of preparing a liposome vesicle useful in gene delivery or gene therapy, comprising combining a nucleic acid, one or more amphiphile(s) of the present invention, and a neutral lipid, thereby forming a bi- or multi-lamellar liposome vesicle.

The compositions and methods of the invention may relate to antisense oligonucleotides that are designed to target specific genes and, consequently, inhibit their expression. In other words, in certain embodiment, a composition of the invention delivers an oligonucleotide that suppress the expression of a gene in the patient or cell. In addition, this delivery system may be a suitable carrier for other gene-targeting oligonucleotides, such as ribozymes, triple-helix-forming oligonucleotides or oligonucleotides exhibiting non-sequence specific binding to a particular protein or other intracellular molecules. For example, the genes of interest may include retroviral or viral genes, drug-resistance genes, oncogenes, genes involved in the inflammatory response, cellular adhesion genes, hormone genes, and abnormally overexpressed genes involved in gene regulation.

Below the present invention is described by reference to specific embodiments. This description is not meant to limit the scope of the invention, but to convey the essence of the invention. Additional embodiments may be readily envisioned by one of ordinary skill in the art, and such embodiments fall within the scope of the invention.

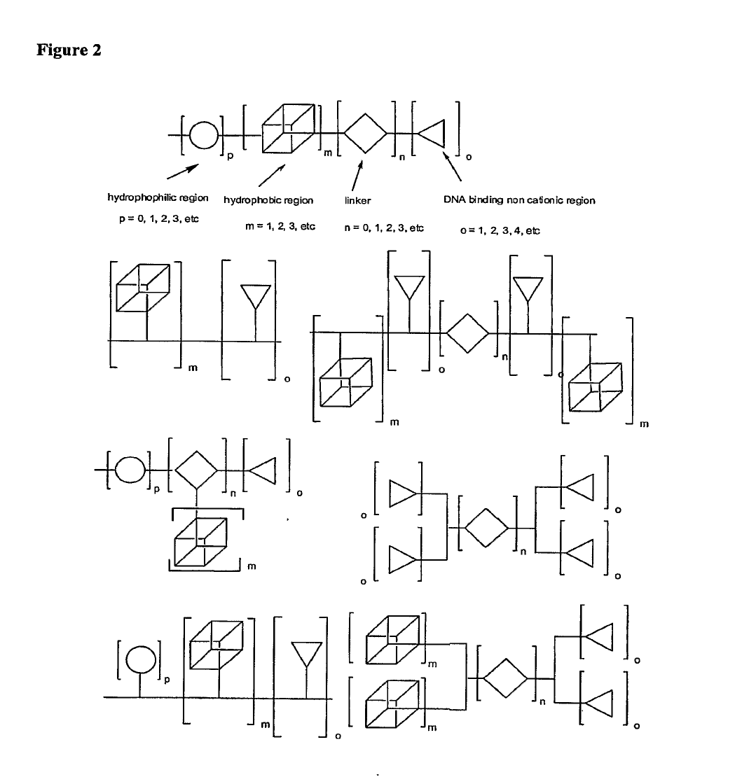

One aspect of the present invention relates to a molecule or macromolecule shown in FIG. 2 that contains at least one DNA binding cationic region, zero or at least one linker regions, and at least one hydrophobic region, zero or at least one hydrophilic regions linked together by covalent bonds, which may be used for the in vitro, ex vivo, or in vivo delivery of nucleic acid to a cell. During the process of delivery to a cell the cationic molecule or macromolecule is transformed from a net cationic entity to a net neutral, net anionic, or zwitterionic entity by a chemical, photochemical, or biological (e.g., enzymatic) reaction.

In certain instances, the aforementioned macromolecule is a homopolymer or heteropolymer (e.g., di-block, multi-block, random co-polymer).

In certain instances, the invention relates to the aforementioned molecule or macromolecule suitable for the delivery of nucleic acids, which molecule or macromolecule undergoes the aforementioned transformation via a photochemical reaction, which reaction is a single- or multi-photon reaction.

In certain instances, the invention relates to the aforementioned molecule or macromolecule suitable for the delivery of nucleic acids, which molecule or macromolecule undergoes the aforementioned transformation via an enzymatic reaction.

In certain instances, the invention relates to the aforementioned molecule or macromolecule, wherein the enzyme is an esterase.

In certain instances, the invention relates to the aforementioned molecule or macromolecule suitable for the delivery of nucleic acids, which molecule or macromolecule undergoes the aforementioned transformation via a redox reaction.

In certain instances, the invention relates to the aforementioned molecule or macromolecule suitable for the delivery of nucleic acids, which molecule or macromolecule undergoes the aforementioned transformation via a temperature change.

In certain instances, the invention relates to the aforementioned molecule or macromolecule suitable for the delivery of nucleic acids, which molecule or macromolecule undergoes the aforementioned transformation via a change in ionic strength.

In certain instances, the invention relates to the aforementioned molecule or macromolecule suitable for the delivery of nucleic acids, which molecule or macromolecule undergoes the aforementioned transformation via a change in pH.

In certain instances, the invention relates to the aforementioned molecule or macromolecule which is tethered to a surface.

In certain instances, the invention relates to the aforementioned molecule or macromolecule further comprising a targeting moiety for a cell or tissue.

In certain instances, the invention relates to the aforementioned molecule or macromolecule further comprising a targeting moiety for the nucleus of a cell.

In certain instances, the invention relates to the aforementioned molecule or macromolecule further comprising a natural peptide or charged peptide or synthetic polymer that destabilizes cell membranes.

In certain instances, the invention relates to the aforementioned molecule or macromolecule further comprising a linker that is neutral, cationic, anionic, and/or zwitterionic.

In certain instances, the invention relates to the aforementioned molecule or macromolecule further comprising a hydrophilic unit that is a hydrophilic polymer (e.g., polyethylene glycol, polyacrylic acids, polyvinyl alcohol) or a small molecule (e.g., tetraethylene glycol, sugar, succinic acid, glycine, glycerol, spermine).

In certain instances, the invention relates to the aforementioned molecule or macromolecule that forms a gel or crosslinked network in aqueous or non-aqueous solution, which gel/crosslinked network is suitable for the delivery of nucleic acids.

Another aspect of the invention relates to a gel/crosslinked network, useful for the delivery of nucleic acids to a cell, formed by a photochemical reaction, enzymatic reaction, an oxidation reaction, a chemical reaction, a pH change, a temperature change, an ionic strength change, a non-covalent interaction(s) with another polymer(s) or molecule(s), or a change in molecule(s) or macromolecule(s) concentration.

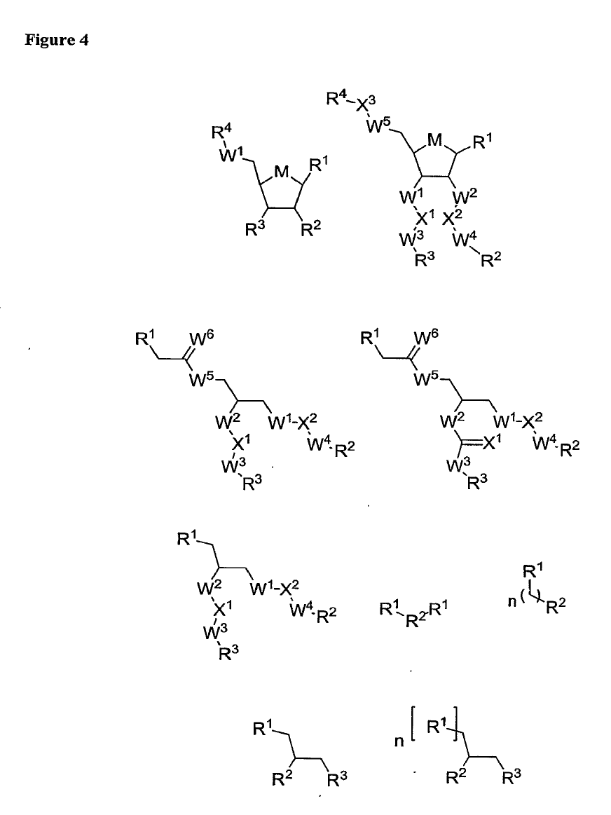

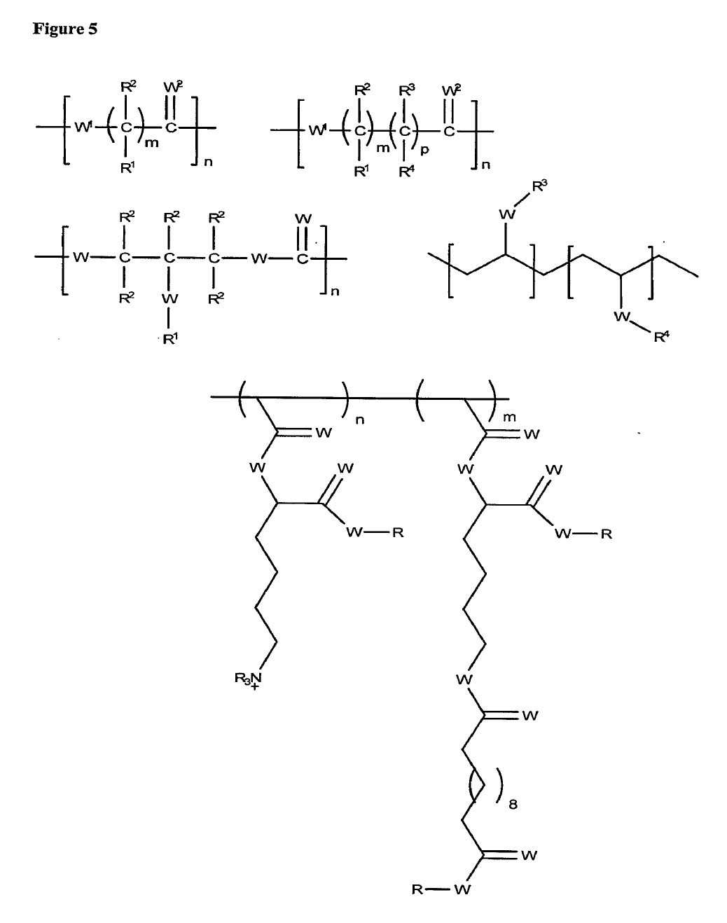

Another aspect of the invention relates to a molecule or macromolecule as shown in FIG. 4 or 5.

In certain instances, the invention relates to the aforementioned macromolecule, wherein the macromolecule is a homopolymer, random copolymer, or block copolymer.

In certain instances, the invention relates to the aforementioned macromolecule, wherein R¹ is at least one non-cationic DNA binding moiety selected from the group consisting of nucleoside, nucleobase, aromatic compound, polyaromatic compound, aliphatic compound, carbohydrate, amino acid, peptide, PNA, and pseudo peptide.

In certain instances, the invention relates to the aforementioned macromolecule wherein R¹ is one or more of the same or different non-cationic DNA binding moiety selected from the group consisting of a nucleoside, nucleobase, aromatic compound, polyaromatic compound, aliphatic compound, carbohydrate, amino acid, and peptide.

In certain instances, the invention relates to the aforementioned macromolecule wherein R¹ is one or more of the same or different cationic DNA binding moiety selected from the group consisting of a primary amine, secondary amine, tertiary amine, quaternary amine (e.g, choline), or molecule(s) possessing more than one cationic amine (e.g., Lys, spermine).

In certain instances, the invention relates to the aforementioned macromolecule, wherein one or more of R¹, R², R³, R⁴, and R⁵ contains a functional group that upon a chemical, photochemical, or biological reaction undergoes a transformation rendering the molecule or macromolecule a neutral, anionic, or zwitterionic molecule or macromolecule.

In certain instances, the invention relates to the aforementioned macromolecule, wherein one or more of R¹, R², R³, R⁴, and R⁵ contains a functional group, such as an ester, that upon a biological reaction transform the molecule(s) or macromolecule(s) to a neutral, anionic, or multi-anionic molecule or macromolecule.

In certain instances, the invention relates to the aforementioned macromolecule wherein one or more of R¹, R², R³, R⁴, and R⁵ contains a functional group selected from the group consisting of phosphate and sulfonate.

In certain instances, the invention relates to the aforementioned macromolecule, wherein one or more of R¹, R², R³, R⁴, and R⁵ contains a functional group, such as an photocleavable ester (e.g., o-nitrobenzyl ester or BHC ester), that upon a photochemical reaction transforms the molecule(s) or macromolecule(s) to a neutral, anionic, or multi-anionic molecule or macromolecule.

In certain instances, the invention relates to the aforementioned macromolecule wherein R², R³, R⁴, and R⁵ are a straight or branched chain ester of 2-50 carbon atoms wherein the chain is fully saturated, fully unsaturated or any combination thereof.

In certain instances, the invention relates to the aforementioned macromolecule, wherein one or more of R², R³, R⁴, and R⁵ is the same or different straight or branched chain ester of 2-50 carbon atoms, wherein the chain is fully saturated, fully unsaturated or any combination thereof, and wherein one or more of R², R³, R⁴, and R⁵ is a —H, —OH, methoxy, amine, or thiol.

In certain instances, the invention relates to the aforementioned macromolecule wherein R², R³, R⁴, and R⁵ are a straight or branched chain ether of 2-50 carbon atoms wherein the chain is fully saturated, fully unsaturated or any combination thereof.

In certain instances, the invention relates to the aforementioned macromolecule wherein one or more of R², R³, R⁴, and R⁵ is the same or different straight or branched chain ether of 2-50 carbon atoms wherein the chain is fully saturated, fully unsaturated or any combination thereof, and wherein one or more of R², R³, R⁴, and R⁵ is a —H, —OH, methoxy, amine, or thiol.

In certain instances, the invention relates to the aforementioned macromolecule wherein R², R³, R⁴, and R⁵ are a straight or branched chain silane of 2-50 carbon atoms wherein the chain is fully saturated, fully unsaturated or any combination thereof.

In certain instances, the invention relates to the aforementioned macromolecule wherein one or more of R², R³, R⁴, and R⁵ is the same or different straight or branched chain silane of 2-50 carbon atoms, wherein the chain is fully saturated, fully unsaturated or any combination thereof, and wherein one or more of R², R³, R⁴, and R⁵ is a —H, —OH, amine, thiol, or methoxy.

In certain instances, the invention relates to the aforementioned macromolecule wherein R², R³, R⁴, and R⁵ are a straight or branched chain amide of 2-50 carbon atoms, wherein the chain is fully saturated, fully unsaturated or any combination thereof.

In certain instances, the invention relates to the aforementioned macromolecule wherein one or more of R², R³, R⁴, and R⁵ is the same or different straight or branched chain amide of 2-50 carbon atoms, wherein the chain is fully saturated, fully unsaturated or any combination thereof, and wherein one or more of R², R³, R⁴, and R⁵ is a —H, —OH, amine, thiol, or methoxy.

In certain instances, the invention relates to the aforementioned macromolecule wherein R², R³, R⁴, and R⁵ are a straight or branched chain urea of 2-50 carbon atoms, wherein the chain is fully saturated, fully unsaturated or any combination thereof.

In certain instances, the invention relates to the aforementioned macromolecule wherein one or more of R², R³, R⁴, and R⁵ is the same or different straight or branched chain urea of 2-50 carbon atoms, wherein the chain is fully saturated, fully unsaturated or any combination thereof, and wherein one or more of R², R³, R⁴, and R⁵ is a —H, —OH, amine, thiol, or methoxy.

In certain instances, the invention relates to the aforementioned macromolecule wherein R², R³, R⁴, and R⁵ are a straight or branched chain urethane of 2-50 carbon atoms wherein the chain is fully saturated, fully unsaturated or any combination thereof.

In certain instances, the invention relates to the aforementioned macromolecule wherein one or more of R², R³, R⁴, and R⁵ is the same or different straight or branched chain urethane of 2-50 carbon atoms, wherein the chain is fully saturated, fully unsaturated or any combination thereof, and wherein one or more of R², R³, R⁴, and R⁵ is a —H, —OH, amine, thiol, or methoxy.

In certain instances, the invention relates to the aforementioned macromolecule wherein R², R³, R⁴, and R⁵ are a straight or branched chain carbonate of 2-50 carbon atoms, wherein the chain is fully saturated, fully unsaturated or any combination thereof.

In certain instances, the invention relates to the aforementioned macromolecule wherein one or more of R², R³, R⁴, and R⁵ is the same or different straight or branched chain carbonate of 2-50 carbon atoms, wherein the chain is fully saturated, fully unsaturated or any combination thereof, and wherein one or more of R², R³, R⁴, and R⁵ is a —H, —OH, amine, thiol, or methoxy.

In certain instances, the invention relates to the aforementioned macromolecule wherein R², R³, R⁴, and R⁵ are a straight or branched chain sulfate of 2-50 carbon atoms, wherein the chain is fully saturated, fully unsaturated or any combination thereof.

In certain instances, the invention relates to the aforementioned macromolecule wherein one or more of R², R³, R⁴, and R⁵ is the same or different straight or branched chain sulfate of 2-50 carbon atoms, wherein the chain is fully saturated, fully unsaturated or any combination thereof, and wherein one or more of R², R³, R⁴, and R⁵ is a —H, —OH, amine, thiol, or methoxy.

In certain instances, the invention relates to the aforementioned macromolecule wherein R², R³, R⁴, and R⁵ are a straight or branched chain thio-urethane of 2-50 carbon atoms, wherein the chain is fully saturated, fully unsaturated or any combination thereof.

In certain instances, the invention relates to the aforementioned macromolecule wherein one or more of R², R³, R⁴, and R⁵ is the same or different straight or branched chain thio-urethane of 2-50 carbon atoms, wherein the chain is fully saturated, fully unsaturated or any combination thereof, and wherein one or more of R², R³, R⁴, and R⁵ is a —H, —OH, amine, thiol, or methoxy.

In certain instances, the invention relates to the aforementioned macromolecule wherein R², R³, R⁴, and R⁵ are a straight or branched chain amine of 2-50 carbon atoms, wherein the chain is fully saturated, fully unsaturated or any combination thereof.

In certain instances, the invention relates to the aforementioned macromolecule wherein one or more of R², R³, R⁴, and R⁵ is the same or different straight or branched chain amine of 2-50 carbon atoms, wherein the chain is fully saturated, fully unsaturated or any combination thereof, and wherein one or more of R², R³, R⁴, and R⁵ is a —H, —OH, amine, thiol, or methoxy.

In certain instances, the invention relates to the aforementioned macromolecule wherein R², R³, R⁴, and R⁵ are a straight or branched chain phosphate of 2-50 carbon atoms wherein the chain is fully saturated, fully unsaturated or any combination thereof.

In certain instances, the invention relates to the aforementioned macromolecule wherein one or more of R², R³, R⁴, and R⁵ is the same or different straight or branched chain phosphate of 2-50 carbon atoms wherein the chain is fully saturated, fully unsaturated or any combination thereof and wherein one or more of R², R³, R⁴, and R⁵ is a —H, —OH, amine, thiol, or methoxy.

In certain instances, the invention relates to the aforementioned macromolecule wherein R², R³, R⁴, and R⁵ are a straight or branched chain thiophosphate of 2-50 carbon atoms wherein the chain is fully saturated, fully unsaturated or any combination thereof.

In certain instances, the invention relates to the aforementioned macromolecule wherein one or more of R², R³, R⁴, and R⁵ is the same or different straight or branched chain thio-phosphate of 2-50 carbon atoms wherein the chain is fully saturated, fully unsaturated or any combination thereof, and wherein one or more of R², R³, R⁴, and R⁵ is a —H, —OH, amine, thiol, or methoxy.

In certain instances, the invention relates to the aforementioned macromolecule wherein R², R³, R⁴, and R⁵ are a straight or branched chain boranophosphate of 2-50 carbon atoms wherein the chain is fully saturated, fully unsaturated or any combination thereof.

In certain instances, the invention relates to the aforementioned macromolecule wherein one or more of R², R³, R⁴, and R⁵ is the same or different straight or branched chain acetal of 2-50 carbon atoms wherein the chain is fully saturated, fully unsaturated or any combination thereof, and wherein one or more of R², R³, R⁴, and R⁵ is a —H, —OH, amine, thiol, or methoxy.

In certain instances, the invention relates to the aforementioned macromolecule wherein R², R³, R⁴, and R⁵ are a straight or branched chain acetal of 2-50 carbon atoms, wherein the chain is fully saturated, fully unsaturated or any combination thereof.

In certain instances, the invention relates to the aforementioned macromolecule wherein one or more of R², R³, R⁴, and R⁵ is the same or different straight or branched chain boranophosphate of 2-50 carbon atoms, wherein the chain is fully saturated, fully unsaturated or any combination thereof, and wherein one or more of R², R³, R⁴, and R⁵ is a —H, —OH, amine, thiol, or methoxy.

In certain instances, the invention relates to the aforementioned macromolecule wherein R², R³, R⁴, and R⁵ are a straight or branched chain thio-urea of 2-50 carbon atoms, wherein the chain is fully saturated, fully unsaturated or any combination thereof.

In certain instances, the invention relates to the aforementioned macromolecule wherein one or more of R², R³, R⁴, and R⁵ is the same or different straight or branched chain thio-urea of 2-50 carbon atoms, wherein the chain is fully saturated, fully unsaturated or any combination thereof, and wherein one or more of R², R³, R⁴, and R⁵ is a —H, —OH, amine, thiol, or methoxy.

In certain instances, the invention relates to the aforementioned macromolecule wherein R², R³, R⁴, and R⁵ are a straight or branched chain thio-ether of 2-50 carbon atoms, wherein the chain is fully saturated, fully unsaturated or any combination thereof.

In certain instances, the invention relates to the aforementioned macromolecule wherein one or more of R², R³, R⁴, and R⁵ is the same or different straight or branched chain thio-ether of 2-50 carbon atoms wherein the chain is fully saturated, fully unsaturated or any combination thereof, and wherein one or more of R², R³, R⁴, and R⁵ is a —H, —OH, anine, thiol, or methoxy.

In certain instances, the invention relates to the aforementioned macromolecule wherein R², R³, R⁴, and R⁵ are a straight or branched chain thio-ester of 2-50 carbon atoms, wherein the chain is fully saturated, fully unsaturated or any combination thereof.

In certain instances, the invention relates to the aforementioned macromolecule wherein one or more of R², R³, R⁴, and R⁵ is the same or different straight or branched chain thio-ester of 2-50 carbon atoms wherein the chain is fully saturated, fully unsaturated or any combination thereof and wherein one or more of R₂, R₃, R₄, and R₅ is a —H, —OH, amine, thiol, or methoxy.

In certain instances, the invention relates to the aforementioned macromolecule wherein R², R³, R⁴, and R⁵ are a straight or branched chain of 2-50 carbon atoms wherein the chain is fully saturated, fully unsaturated or any combination thereof.

In certain instances, the invention relates to the aforementioned macromolecule wherein one or more of R², R³, R⁴, and R⁵ is the same or different straight or branched chain of 2-50 carbon atoms wherein the chain is fully saturated, fully unsaturated or any combination thereof and wherein one or more of R², R³, R⁴, and R⁵ is a —H, —OH, amine, thiol, or methoxy.

In certain instances, the invention relates to the aforementioned macromolecule wherein the chains are independently hydrocarbons, fluorocarbons, halocarbons, alkenes, or alkynes or any combination thereof.

In certain instances, the invention relates to the aforementioned macromolecule wherein one or more of R², R³, R⁴, and R⁵ chains are polypeptide(s) or contain at least one amino acid, wherein one or more R², R³, R⁴, and R⁵ is a chain as described above.

In certain instances, the invention relates to the aforementioned macromolecule, wherein one or more of the chains contains a disulfide bond or linkage.

In certain instances, the invention relates to the aforementioned macromolecule wherein one or more of the chains contains a linkage susceptible to cleavage by a change in pH, light, or an enzyme.

In certain instances, the invention relates to the aforementioned macromolecule wherein the chains are amino acid(s) or polypeptide(s) combined with one or more chain moieties selected from the group consisting of hydrocarbons, fluorocarbons, halocarbons, alkenes, and alkynes and any combination thereof.

In certain instances, the invention relates to the aforementioned macromolecule wherein one chain or more of the chains contains one or more ionic, photo, covalent crosslinkable group.

In certain instances, the invention relates to the aforementioned macromolecule, wherein the straight or branched chains comprise the same number of carbons or different, wherein one or more of R², R³, R⁴, and R⁵ comprises any combination of the linkers selected from the group consisting of ester, silane, urea, amide, amine, carbamate, urethane, thio-urethane, carbonate, thio-ether, thio-ester, sulfate, sulfoxide, nitroxide, phosphate and ether.

In certain instances, the invention relates to the aforementioned molecule or macromolecule wherein at least one chain terminates with a functional group selected from the group consisting of amine, thiol, amide, carboxylic acid, phosphate, sulphate, hydroxide, and selenol.

In certain instances, the invention relates to the aforementioned molecule or macromolecule wherein at least one chain terminates with a functional group that can be subsequently transformed from a neutral species to an anionic or zwitterionic group.

In certain instances, the invention relates to the aforementioned molecule or macromolecule wherein at least one chain terminates with a functional group selected from the group consisting of protected carboxylic acids and protected phosphates, which are protected with a group that can be liberated by a chemical, biological, or photochemical group.

In certain instances, the invention relates to the aforementioned molecule or macromolecule wherein at least one chain terminates with one or more Ser, Tyr, or Thr or at least one amino acid (including a peptide) that is susceptible to a biological reaction, such as phosphorylation.

In certain instances, the invention relates to the aforementioned molecule or macromolecule wherein the preferred chain length is about 6-24.

In certain instances, the invention relates to the aforementioned molecule or macromolecule, wherein M is O, S, N—H, or N—R, wherein R is —H, CH₂, C(R)₂, a chain as defined above, Se or any isoelectronic species of oxygen.

In certain instances, the invention relates to the aforementioned molecule or macromolecule wherein the cyclic structure is of 4 or more atoms or bicyclic.

In certain instances, the invention relates to the aforementioned molecule or macromolecule wherein W is O, S, N—H, or N—R, wherein R is —H, CH₂, C(R)₂, a chain as defined above, Se or any isoelectronic species of oxygen, optionally comprising XYZ.

In certain instances, the invention relates to the aforementioned molecule or macromolecule wherein W is a phosphonate, phosphate, boronophosphate, thiophosphate, or selenophosphate.

In certain instances, the invention relates to the aforementioned molecule or macromolecule wherein X is a phosphonate, phosphate, boronophosphate, thiophosphate, or selenophosphate.

In certain instances, the invention relates to the aforementioned molecule or macromolecule, wherein one or more of R², R³, R⁴, and R⁵ is hydroxide, N-succinyl derivative, amino acid, carbohydrate, nucleic acid, multiple amines, multiple hydroxides, cyclic amine, polyamine, polyether, polyester or tertiary, secondary or primary amine, optionally comprising a chain of 1-20 carbons.

In certain instances, the invention relates to the aforementioned molecule or macromolecule wherein an antibody or single chain antibody is attached to a chain as described above.

In certain instances, the invention relates to the aforementioned molecule or macromolecule wherein a nucleotide is attached to a chain as described above.

In certain instances, the invention relates to the aforementioned molecule or macromolecule wherein a nucleoside is attached to a chain as described above.

In certain instances, the invention relates to the aforementioned molecule or macromolecule wherein an oligonucleotide is attached to a chain as described above.

In certain instances, the invention relates to the aforementioned molecule or macromolecule wherein a contrast agent is attached to a chain as described above.

In certain instances, the invention relates to the aforementioned molecule or macromolecule wherein a ligand for a biological receptor is attached to a chain as described above.

In certain instances, the invention relates to the aforementioned molecule or macromolecule wherein a pharmaceutical agent is attached to a chain as described above.

In certain instances, the invention relates to the aforementioned molecule or macromolecule wherein a carbohydrate is attached to a chain as described above.

In certain instances, the invention relates to the aforementioned molecule or macromolecule wherein said contrast agent is a PET or MRI agent, such as Gd(DPTA).

In certain instances, the invention relates to the aforementioned molecule or macromolecule wherein an iodated compound useful for X-ray imaging is attached.

In certain instances, the invention relates to the aforementioned molecule or macromolecule, wherein a carbohydrate is lactose, galactose, glucose, mannose, sialic acid fucose, fructose, manose, sucrose, cellobiose, nytrose, triose, dextrose, trehalose, maltose, galactosamine, glucosamine, galacturonic acid, glucuronic acid, gluconic acid, or lactobionic acid.

In certain instances, the invention relates to the aforementioned molecule or macromolecule wherein a stereochemical center is present that affords chiral compounds.

In certain instances, the invention relates to the aforementioned molecule or macromolecule, wherein any of the above compositions are covalently attached to form a compound similar to a geminal lipid.

In certain instances, the invention relates to the aforementioned molecule or macromolecule wherein any of the above compositions have both of their chain groups attached in a cyclical fashion to another lipid, such as in a bolalipid.

Another aspect of the present invention relates to a composition comprising one of the aforementioned compounds mixed from 0.1-99.9% with a known cationic, anionic or zwitterionic molecule or macromolecule, such as DOPE, DLPC, DMPC, DPPC, DSPC, DOPC, DMPE, DOPE, DPPE, DMPA-Na, DMRPC, DLRPC, DARPC, or similar catonic, anionic, or zwitterionic amphiphiles.

In certain instances, the invention relates to the aforementioned macromolecule that forms a supramolecular structure, such as a liposome (multilamellar, single lamellar, giant), helix, disc, tube, fiber, torus, hexagonal phase, micelle, gel phase, reverse micelle, microemulsion or emulsion.

In certain instances, the invention relates to the aforementioned composition that forms a microemulsion, nanoemulsion, or emulsion.

Another aspect of the present invention relates to a supramolecular structure formed from a combination of an aforementioned compound with from 0.1-99.9% of a known material, such as DPPC, DMPC, PEGylated DPPC, DOPE, DLPC, DMPC, DPPC, DSPC, DOPC, DMPE, DOPE, DPPE, DMPA-Na, DMRPC, DLRPC, DARPC, or similar catonic, anionic, or zwitterionic amphiphiles, fatty acids, cholesterol, fluorescently labeled phospholipids, ether lipids, or sphingolipids.

In certain instances, the invention relates to the aforementioned macromolecule tethered to a surface, wherein the surface is selected from the group consisting of glass, mica, polymer, metal, metal alloy, ceramic, oxide, and the like.

Another aspect of the present invention relates to the aforementioned composition or supramolecular structure in an aqueous solution, wherein said aqueous solution comprises water, buffered aqueous media, saline, buffered saline, aqueous solutions of amino acids, aqueous solutions of sugars, aqueous solutions of vitamins, aqueous solutions of carbohydrates or a combination of any of them.

Another aspect of the present invention relates to the aforementioned composition or supramolecular structure in solution, comprising water, buffered aqueous media, saline, buffered saline, aqueous solutions of amino acids, aqueous solutions of sugars, aqueous solutions of vitamins, aqueous solutions of carbohydrates or a combination of any of them; and DMSO, ethanol, methanol, THF, dichloromethane, DMF or a combination of any of them.

Another aspect of the present invention relates to the aforementioned composition or supramolecular structure in the form of a particle, foam, gel, or supramolecular assembly. The present invention also relates to a method for preparing a liposome comprising a molecule or supramolecular structure of the present invention, comprising the steps of forming a film of a lipid on a glass coverslip; and incubating it in a sucrose solution comprising said molecule or supramolecular structure. The present invention also relates to a method for preparing a liposome comprising a molecule or supramolecular structure of the present invention, comprising the steps of depositing a thin film of a lipid on the inside of a round bottom flask; and rehydrating said thin film at a temperature above its phase transition temperature using an aqueous solution comprising said molecule or supramolecular structure. The present invention also relates to a method for preparing a liposome comprising a molecule or supramolecular structure of the present invention, comprising the step of sonicating hydrated lipids in the presence of an aqueous solution comprising said molecule or supramolecular structure. In certain embodiments, the liposomes are formed using an extrusion, sonication or vortexing method in the presence or absence of nucleic acids. In certain embodiments, the aforementioned compositions are modified in order to destabilize in acidic, basic, or neutral environments. In certain embodiments, the aforementioned compositions are modified in order to destabilize in cold, warm, or ultrasonic environments. Any of the aforementioned compositions optionally comprises a cationic molecule or macromolecule.

Another aspect of the present invention relates to a method for delivering to a cell a nucleic acid, comprising contacting a cell with any one of the aforementioned compositions or supramolecular structures.

Another aspect of the present invention relates to a method for transfection, comprising contacting a cell with any one of the aforementioned compositions or supramolecular structures comprising a nucleic acid.

Another aspect of the present invention relates to the aforementioned method for nucleic acid delivery and transfection, wherein said composition or supramolecular structure comprising a nucleic acid further comprises from 0.1-99.9% of a compound selected from the group consisting of DPPC, DMPC, PEGylated DPPC, DPPC, DOPE, DLPC, DMPC, DPPC, DSPC, DOPC, DMPE, DOPE, DPPE, DMPA-Na, DMRPC, DLRPC, DARPC, or catonic, anionic, or zwitterionic amphiphiles, fatty acids, cholesterol, fluorescenctly labeled phospholipids, lipids, and sphingolipids.

In certain instances, the invention relates to the aforementioned composition comprising a nucleic acid, wherein the nucleic acid comprises a DNA sequence encoding a genetic marker selected from the group consisting of luciferase gene, beta-galactosidase gene, hygromycin resistance, neomycin resistance, and chloramphenicol acetyl transferase.

In certain instances, the invention relates to the aforementioned composition comprising a nucleic acid, wherein said nucleic acid comprises a DNA sequence encoding a protein selected from the group consisting of low density lipoprotein receptors, coagulation factors, gene suppressors of tumors, major histocompatibility proteins, antioncogenes, p16, p53, thymidine kinase, IL2, IL 4, and TNFa.

In certain instances, the invention relates to the aforementioned composition comprising a nucleic acid, wherein the nucleic acid comprises a DNA sequence encoding a viral antigen.

In certain instances, the invention relates to the aforementioned composition comprising a nucleic acid, wherein the nucleic acid comprises a DNA sequence encoding an RNA selected from the group consisting of sense RNA, antisense RNA, and a ribozyme.

In certain instances, the invention relates to the aforementioned composition comprising a nucleic acid, wherein the nucleic acid comprises a DNA sequence encoding lectin, a mannose receptor, a sialoadhesin, or a retroviral transactivating factor.

In certain instances, the invention relates to the aforementioned composition comprising a nucleic acid, wherein the nucleic acid comprises a DNA or RNA sequence of medical interest or relevance.

Another aspect of the invention relates to a method of transfecting cells in vitro, ex vivo, or in vivo, comprising contacting a cell with any one of the aforementioned compositions under conditions, wherein said composition enters said cells, and the nucleic acid of said composition is released.

Another aspect of the invention relates to an in vitro, ex vivo, or in vitro method of transfecting cells bearing a receptor recognizing a targeting moiety, comprising contacting a cell bearing a receptor recognizing a targeting moiety with a composition of the invention comprising a nucleic acid, under conditions wherein said composition enters said cells, and the nucleic acid of said composition is released.

Another aspect of the invention relates to an in vitro, ex vivo, or in vitro method of transfecting cells, wherein the cells are human cells, including embryonic stem cells, animal cells, plant cells, insect cells, immortal cells, or genetically engineered cells.

Another aspect of the invention relates to the use of transfected cells for treating a disease or repairing an injured tissue, organ, or bone.

Another aspect of the invention relates to a method of treating a disease or repairing an injured tissue, organ, or bone, comprising administering to an patient in need thereof a composition of the present invention comprising a nucleic acid.

Another aspect of the invention relates to a method of treating cancer, comprising administering to a patient in need thereof a composition of the present invention comprising a nucleic acid.

Another aspect of the invention relates to a method of treating or correcting a genetic defect, comprising administering to a patient in need thereof a composition of the present invention comprising a nucleic acid.

Another aspect of the invention relates to a method of treating a medical condition, comprising administering to a patient in need thereof a composition of the present invention comprising a nucleic acid.

Another aspect of the invention relates to a method of crop management or food manufacturing, comprising administering to a patient in need thereof a composition of the present invention comprising a nucleic acid.

### Compounds of the Invention

One aspect of the present invention relates to a compound represented by Formula I:

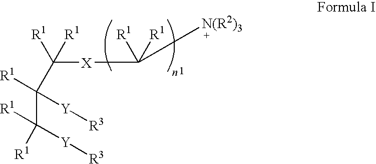

wherein

X represents

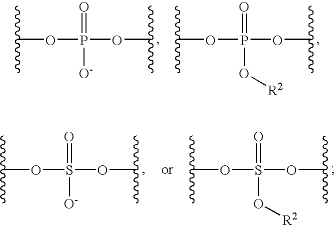

R¹ represents independently for each occurrence H, alkyl, or halogen;

R² represents independently for each occurrence H, alkyl, alkenylalkyl, aryl, or aralkyl;

R³ represents independently for each occurrence alkyl, alkenylalkyl, aryl, aralkyl,

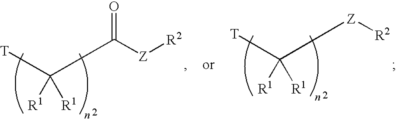

n¹ and n² represent independently for each occurrence an integer from 1-50;

Y and Z represent independently for each occurrence O or —N(R²)—; and

T represents independently for each occurrence —C(R²)₂—, or —C(═O)—.

In certain embodiments, the present invention relates to the aforementioned compound, wherein X is

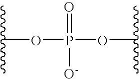

, R¹ is H, n¹ is 2, n² is 10, Y is O, Z is O, and T is —C(═O)—.

In certain embodiments, the present invention relates to the aforementioned compound, wherein X is

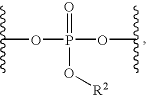

R¹ is H, n¹ is 2, n² is 10, Y is O, Z is O, and T is —C(═O)—.

In certain embodiments, the present invention relates to the aforementioned compound, wherein said compound of Formula I is:

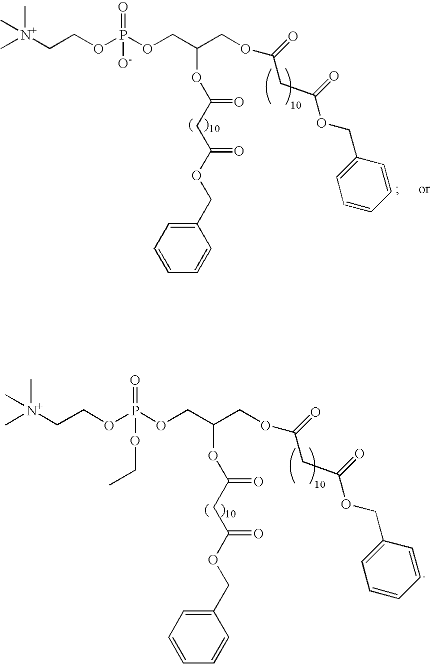

Another aspect of the present invention relates to a compound represented by Formula II:

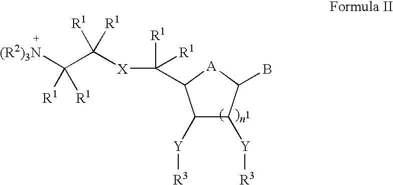

wherein

A represents O, —N(R²)—, or —C(R²)₂—;

B represents a methoxy group, purine base, or pyrimidine base;

X represents

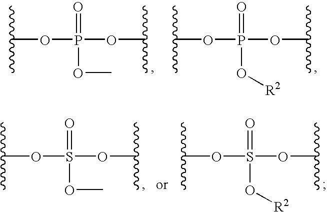

R¹ represents independently for each occurrence H, alkyl, or halogen;

R² represents independently for each occurrence H, alkyl, alkenylalkyl, aryl, or aralkyl;

R³ represents independently for each occurrence alkyl, alkenylalkyl, aryl, aralkyl,

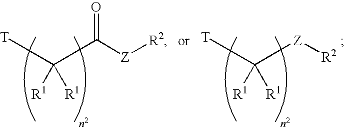

n¹ and n² represent independently for each occurrence an integer from 1-50;

Y and Z represent independently for each occurrence O or —N(R²)—; and

T represents independently for each occurrence —C(R²)₂—, or —C(═O)—.

In certain embodiments, the present invention relates to the aforementioned compound, wherein A is O, B is

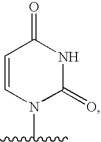

### X is

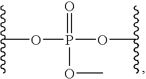

R¹ is H, n¹ is 1, n² is 10, Y is O, Z is O, and T is —C(═O)—.

In certain embodiments, the present invention relates to the aforementioned compound, wherein A is O, B is

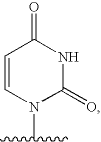

### , X is

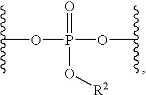

R¹ is H, n¹ is 1, n² is 10, Y is O, Z is O, and T is —C(═O)—.

In certain embodiments, the present invention relates to the aforementioned compound, wherein said compound of Formula II is:

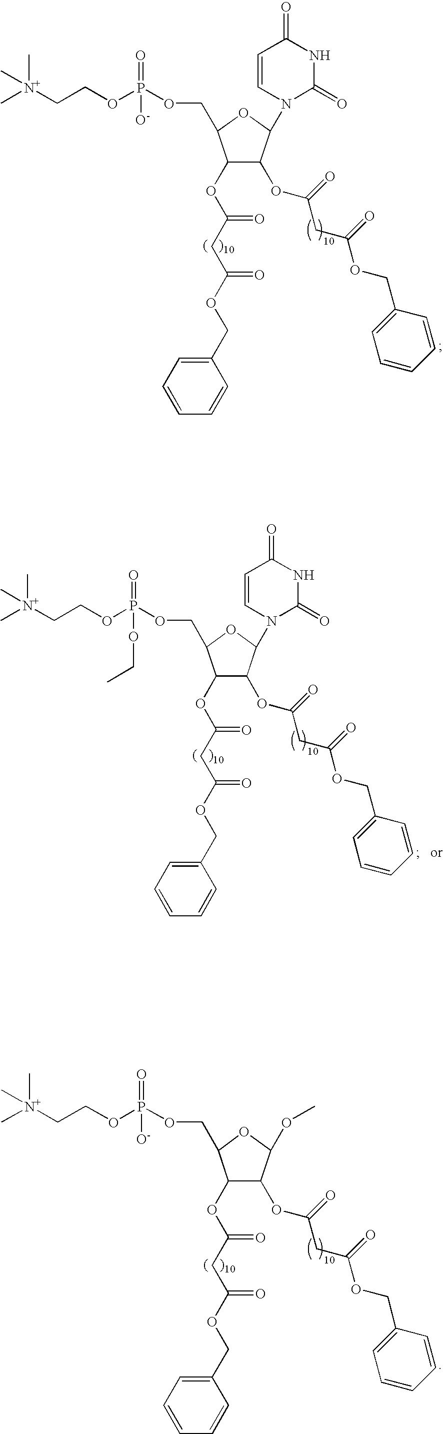

Another aspect of the present invention relates to a compound represented by Formula III:

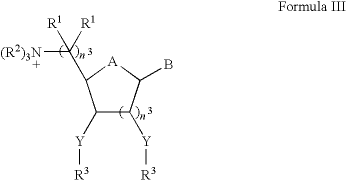

wherein

A represents O, —N(R²)—, or —C(R²)₂—;

B represents a methoxy group, purine base, or pyrimidine base;

R¹ represents independently for each occurrence H, alkyl, or halogen;

R² represents independently for each occurrence H, alkyl, alkenylalkyl, aryl, or aralkyl;

R³ represents independently for each occurrence alkyl, alkenylalkyl, aryl, aralkyl,

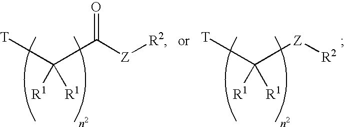

n¹, n², and n³ represent independently for each occurrence an integer from 1-50;

Y and Z represent independently for each occurrence O or —N(R²)—; and

T represents independently for each occurrence —C(R²)₂—, or —C(═O)—.

In certain embodiments, the present invention relates to the aforementioned compound, wherein A is O, B is

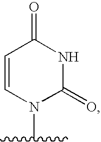

R¹ is H, n¹ is 1, n² is 8, n³ is 1, Y is O, Z is O, and T is —C(═O)—.

In certain embodiments, the present invention relates to the aforementioned compound, wherein said compound of Formula III is:

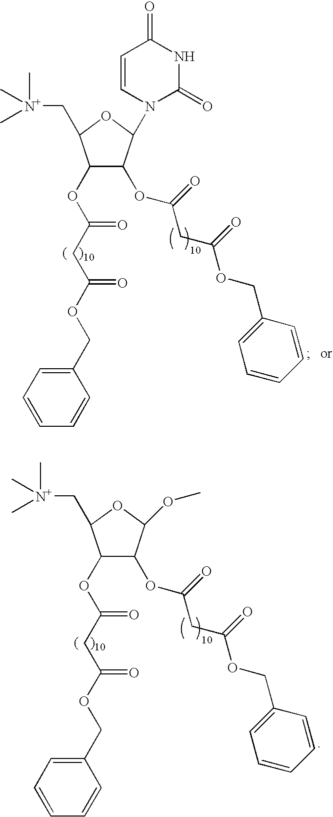

Another aspect of the present invention relates to a compound represented by Formula IV:

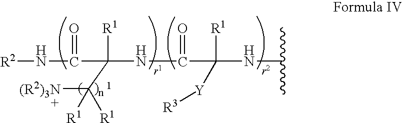

wherein

R¹ represents independently for each occurrence H, alkyl, or halogen;

R² represents independently for each occurrence H, alkyl, alkenylalkyl, aryl, or aralkyl;

R³ represents independently for each occurrence alkyl, alkenylalkyl, aryl, aralkyl,

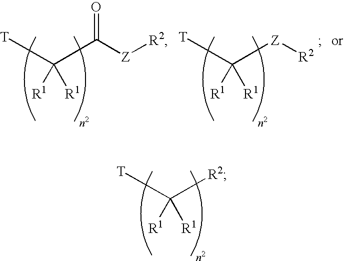

n¹ and n² represent independently for each occurrence an integer from 1-50;

Y and Z represent independently for each occurrence O, —N(R²)—, —O—C(═O)—O—, or —O—(C═O)—N(R²)—;

T represents independently for each occurrence —C(R²)₂—, or —C(═O)—; and

r¹ and r² represent independently for each occurrence an integer from 1-500.

In certain embodiments, the present invention relates to the aforementioned compound, wherein R¹ is H, n¹ is 4, n² is 2, Y is O, Z is O, and T is —C(═O)—.

In certain embodiments, the present invention relates to the aforementioned compound, wherein R¹ is H, n¹ is 4, Y is O, Z is O, and T is —C(═O)—.

In certain embodiments, the present invention relates to the aforementioned compound, wherein said compound of Formula IV is:

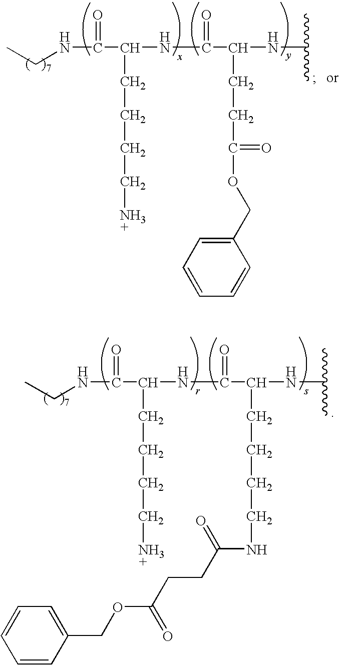

Another aspect of the present invention relates to a compound represented by Formula V:

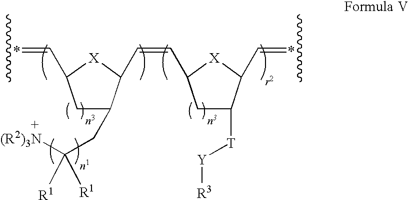

wherein

R¹ represents independently for each occurrence H, alkyl, or halogen;

R² represents independently for each occurrence H, alkyl, alkenylalkyl, aryl, or aralkyl;

R³ represents independently for each occurrence alkyl, alkenylalkyl, aryl, aralkyl,

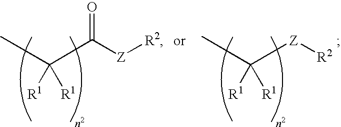

X represents independently for each occurrence O, S, or NR¹;

n¹, n², and n³ represent independently for each occurrence an integer from 1-50;

Y and Z represent independently for each occurrence O or —N(R²)—;

T represents independently for each occurrence —C(R²)₂—, —C(═O)—, or —O—(C═O)—; and

r¹ and r² represent independently for each occurrence an integer from 1-500.

In certain embodiments, the present invention relates to the aforementioned compound, wherein R¹ is H, n¹ is 2, n² is 11, n³ is 1, Y is O, Z is O, and T is C(═O)—.

In certain embodiments, the present invention relates to the aforementioned compound, wherein said compound of Formula V is:

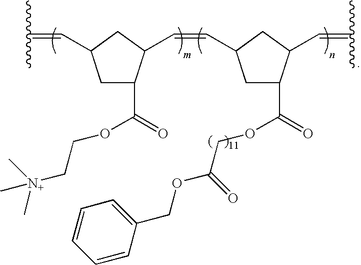

Another aspect of the present invention relates to a compound represented by Formula VI:

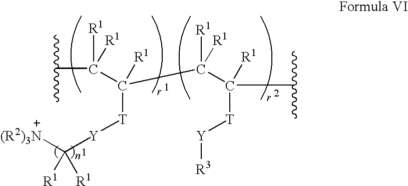

wherein

R¹ represents independently for each occurrence H, alkyl, or halogen;

R² represents independently for each occurrence H, alkyl, alkenylalkyl, aryl, or aralkyl;

R³ represents independently for each occurrence alkyl, alkenylalkyl, aryl, aralkyl,

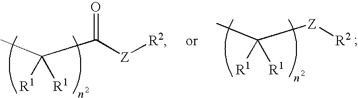

n¹ and n² represent independently for each occurrence an integer from 1-50;

Y and Z represent independently for each occurrence O or —N(R²)—;

T represents independently for each occurrence —C(R²)₂—, —C(═O)—, or —O—(C═O)—; and

r¹ and r² represent independently for each occurrence an integer from 1-500.

In certain embodiments, the present invention relates to the aforementioned compound, wherein R¹ is H, n¹ is 2, n² is 11, Y is O, Z is O, and T is —C(═O)—.

In certain embodiments, the present invention relates to the aforementioned compound, wherein said compound of Formula VI is:

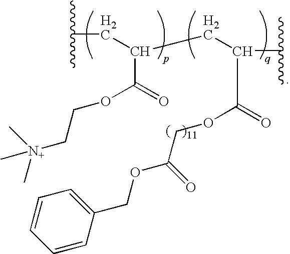

Another aspect of the present invention relates to a compound represented by Formula VII:

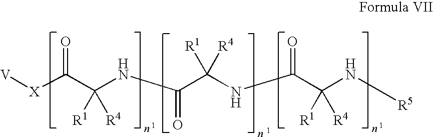

wherein

X represents O, —N(R²)—, —C(═O)—, —C(═O)N(R²)—, —OC(═O)N(R²)—, —N(R²)C(—O)—, or —O—C(═O)—;

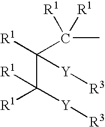

V represents or an optionally substituted saturated or unsaturated cyclopentaphenanthrene ring;

R¹ represents independently for each occurrence H, alkyl, or halogen;

R² represents independently for each occurrence H, alkyl, alkenylalkyl, aryl, or aralkyl;

R³ represents independently for each occurrence alkyl, alkenylalkyl, aryl, aralkyl,

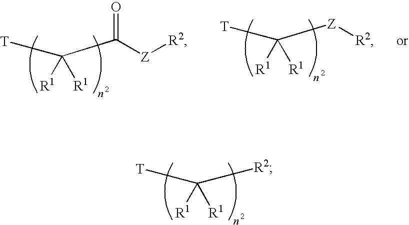

R⁴ represents independently for each occurrence an amino acid side chain;

R⁵ represents independently for each occurrence H, alkyl, alkenylalkyl, aryl, aralkyl, or

### —C(═O)N(R²)—;

n¹ and n² represent independently for each occurrence an integer from 1-50;

Y and Z represent independently for each occurrence O, —N(R²)—, —O—C(═O)—O—, or O—(C═O)—N(R²)—; and

T represents independently for each occurrence —C(R²)₂—, or —C(═O)—.

In certain embodiments, the present invention relates to the aforementioned compound, wherein R¹ is H, n¹ is 1, n² is 8, Y is O, Z is O, and T is C(═O)—.

In certain embodiments, the present invention relates to the aforementioned compound, wherein R¹ is H, n¹ is 1, n² is 8, Y is O, and R³ is alkyl.

In certain embodiments, the present invention relates to the aforementioned compound, wherein V is cholesterol.

In certain embodiments, the present invention relates to the aforementioned compound, wherein said compound of Formula VII is:

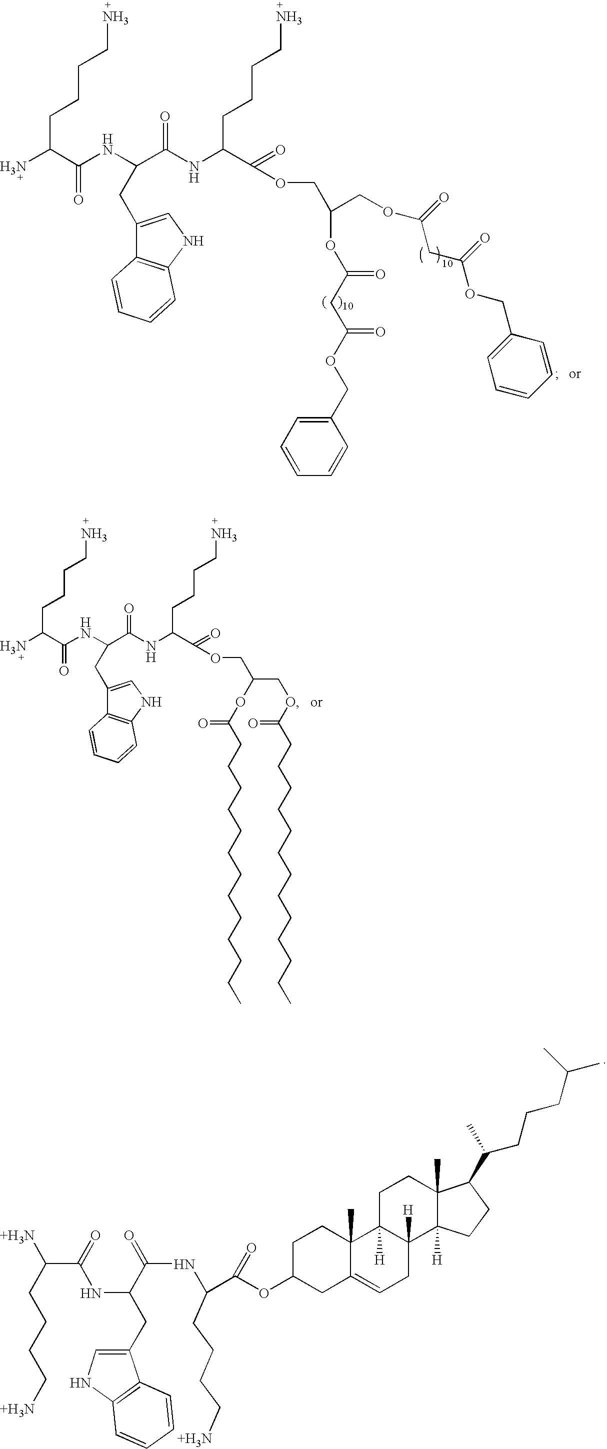

Another aspect of the invention relates to a compound represented by Formula VIII:

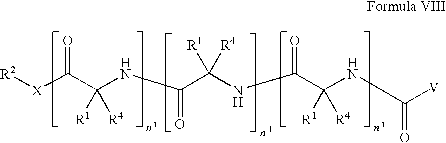

wherein

X represents O, —N(R²)—, —C(═O)—, —C(═O)N(R²)—, —OC(═O)N(R²)—, —N(R²)C(═O)—, or —O—C(═O)—;

V represents

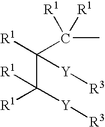

or an optionally substituted saturated or unsaturated cyclopentaphenanthrene ring;

R¹ represents independently for each occurrence H, alkyl, or halogen;

R² represents independently for each occurrence H, alkyl, alkenylalkyl, aryl, or aralkyl;

R³ represents independently for each occurrence alkyl, alkenylalkyl, aryl, aralkyl,

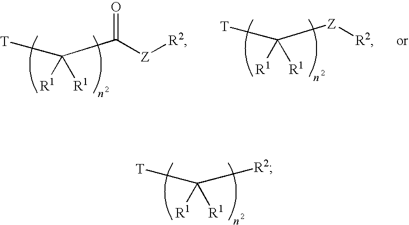

R⁴ represents independently for each occurrence an amino acid side chain;

n¹ and n² represent independently for each occurrence an integer from 1-50;

Y and Z represent independently for each occurrence O, —N(R²)—, —O—C(═O)—O—, or O—(C═O)—N(R²)—; and

T represents independently for each occurrence —C(2)₂—, or —C(═O)—.

Another aspect of the present invention relates to a compound represented by Formula IX:

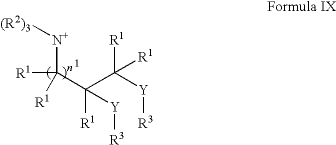

wherein

R¹ represents independently for each occurrence H, alkyl, or halogen;

R² represents independently for each occurrence H, alkyl, alkenylalkyl, aryl, or aralkyl;

R³ represents independently for each occurrence alkyl, alkenylalkyl, aryl, aralkyl,

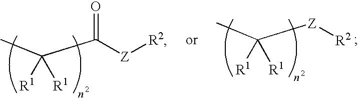

n¹ and n² represent independently for each occurrence an integer from 1-50;

Y and Z represent independently for each occurrence O or —N(R²)—; and

T represents independently for each occurrence —C(R²)₂—, or —C(═O)—. In certain embodiments, the present invention relates to the aforementioned compound, wherein R¹ is H, n¹ is 1, n² is 10, Y is O, Z is O, T is C(═O)—, two instances of R¹ bonded to N are Me, and one instance of R² bonded to N is CH₂CH₂OH.

In certain embodiments, the present invention relates to the aforementioned compound, wherein R¹ is H, n¹ is 1, n² is 10, Y is O, Z is O, T is C(═O)—, one instance of R² bonded to N is Me, and two instances of R² bonded to N are —CH₂CH₂OH. In certain embodiments, the present invention relates to the aforementioned compound, wherein said compound of Formula IX is:

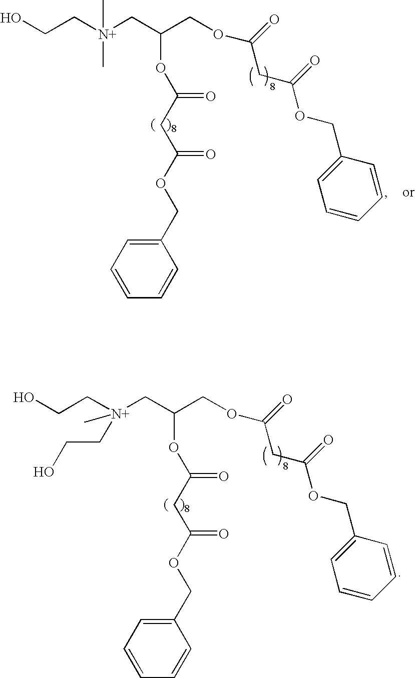

Another aspect of the present invention relates to a compound represented by Formula X:

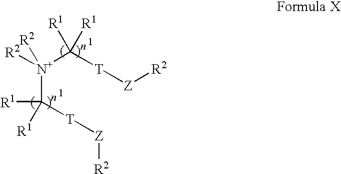

wherein

R¹ represents independently for each occurrence H, alkyl, or halogen;

R² represents independently for each occurrence H, alkyl, alkenylalkyl, aryl, or aralkyl;

n¹ represents independently for each occurrence an integer from 1-50;

Z represents independently for each occurrence O or —N(R²)—; and

T represents independently for each occurrence —C(R²)₂—, or —C(═O)—.

In certain embodiments, the present invention relates to the aforementioned compound, wherein R¹ is H, n¹ is 9, Z is O, T is —C(═O)—.

In certain embodiments, the present invention relates to the aforementioned compound, wherein said compound of Formula X is:

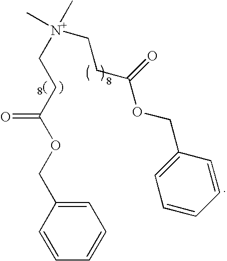

### Methods of the Invention

Gene therapy can be used for treatment of cancer; for example, its utility has been described in the treatment of prostate, colorectal, ovarian, lung, and breast cancer. Gene therapy has been explored for delivery of vaccines for infectious disease, for lysosomal storage disorders, for dendritic cell-based immunotherapy, for controlling hypertension, and for rescuing ischaemic tissues. Gene therapy has also been explored for treating HIV. See Galanis, E.; Vile, R.; Russell, S. J. Crit. Rev. Oncol. Hemat 2001, 38, 177-192; Kim, D.; Martuza, R. L; Zwiebel, J. Nature Med. 2001, 7, 783-789; Culver, K. W.; Blaese, R. M. Trends Genet. 1994, 10, 174-178; Harrington, K. J.; Spitzweg, C.; Bateman, A. R.; Morris, J. C.; Vile, R. G. J. Urology 2001, 166, 1220-1233; Chen, M. J.; Chung-Faye, G. A.; Searle, P. F.; Young, L. S.; Kerr, D. J. Biodrugs 2001, 15, 357-367; Wen, S. F.; Mahavni, V.; Quijano, E.; Shinoda, J.; Grace, M.; Musco-Hobkinson, M. L.; Yang, T. Y.; Chen, Y. T.; Runnenbaum, I.; Horowitz, J.; Maneval, D.; Hutchins, B.; Buller, R. Cancer Gene Ther. 2003, 10, 224-238; Hoang, T.; Traynor, A. M.; Schiller, J. H. Surg. Oncol. 2002, 11, 229-241; Patterson, A.; Harris, A. L. Drugs Aging 1999, 14, 75-90; Clark, K. R.; Johnson, P. R. Curr. Op. Mol. Ther. 2001, 3, 375-384; Yew, N. S.; Cheng, S. H. Curr. Op. Mol. Ther. 2001, 3, 399-406; Jenne, L.; Schuler, G.; Steinkasserer, A. Trends Immunol 2001, 22; Sellers, K. W.; Katovich, M. J.; Gelband, C. H.; Raizada, M. K. Am. J. Med. Sci. 2001, 322, 1-6; Emanueli, C.; Madeddu, P. Brit. J. Pharmacol. 2001, 133, 951-958; and Schnell, M. J. FEMS Microbiol Lett 2001, 200, 123-129.

One aspect of the present invention relates to a method of delivering a nucleic acid to a cell, comprising the step of:

contacting a cell with an effective amount of a mixture comprising a nucleic acid; and a compound of class I, II, III, IV, V, VI, VII, VIII, IX, or X.

In certain embodiments, the present invention relates to the aforementioned method, wherein said nucleic acid is DNA, RNA, plasmid, siRNA, duplex oligonucleotide, single-strand oligonucleotide, triplex oligonucleotide, PNA, or mRNA.

In certain embodiments, the present invention relates to the aforementioned method, wherein said nucleic acid consists of about 10 to about 5000 nucleotides.

In certain embodiments, the present invention relates to the aforementioned method, wherein said nucleic acid is DNA or RNA.

In certain embodiments, the present invention relates to the aforementioned method, wherein said nucleic acid is a DNA or RNA sequence related to a mammalian disease.

In certain embodiments, the present invention relates to the aforementioned method, wherein said nucleic acid is a DNA or RNA sequence related to a cancer. In certain embodiments, the cancer is lung, breast, colon, prostate, or brain cancer.

In certain embodiments, the present invention relates to the aforementioned method, wherein said nucleic acid is DNA.

In certain embodiments, the present invention relates to the aforementioned method, wherein said nucleic acid is a DNA or RNA sequence targeting gene selected from the group consisting of retroviral gene, viral gene, drug resistance gene, oncogene, gene related to inflammatory response, cellular adhesion gene, hormone gene, and abnormally overexpressed gene involved in gene regulation.

In certain embodiments, the present invention relates to the aforementioned method, wherein said nucleic acid is a DNA or RNA sequence related to cancer, viral infection, bacterial infection, lysosomal storage disorder, hypertension, ischaemic disorder, or HIV.

In certain embodiments, the present invention relates to the aforementioned method, wherein said nucleic acid is a DNA sequence encoding a genetic marker selected from the group consisting of luciferase gene, beta-galactosidase gene, hygromycin resistance, neomycin resistance, and chloramphenicol acetyl transferase.

In certain embodiments, the present invention relates to the aforementioned method, wherein said nucleic acid is a DNA sequence encoding a protein selected from the group consisting of low density lipoprotein receptors, coagulation factors, gene suppressors of tumors, major histocompatibility proteins, antioncogenes, p16, p53, thymidine kinase, IL2, IL 4, and TNFa.

In certain embodiments, the present invention relates to the aforementioned method, wherein said nucleic acid is a DNA sequence encoding a viral antigen.

In certain embodiments, the present invention relates to the aforementioned method, wherein said nucleic acid is a DNA sequence encoding an RNA selected from the group consisting of sense RNA, antisense RNA, and ribozyme.

In certain embodiments, the present invention relates to the aforementioned method, wherein said nucleic acid is a DNA sequence encoding a lectin, mannose receptor, sialoadhesin, or retroviral transactivating factor.

In certain embodiments, the present invention relates to the aforementioned method, wherein said cell is an animal cell or plant cell.

In certain embodiments, the present invention relates to the aforementioned method, wherein said cell is a mammalian cell.

In certain embodiments, the present invention relates to the aforementioned method, wherein said cell is a human cell or insect cell.

In certain embodiments, the present invention relates to the aforementioned method, wherein said cell is a human cell.

In certain embodiments, the present invention relates to the aforementioned method, wherein said cell is an embryonic cell or stem cell.

In certain embodiments, the present invention relates to the aforementioned method, wherein said cell is contacted in vivo, in vitro, or ex vivo.

In certain embodiments, the present invention relates to the aforementioned method, wherein said cell is contacted in vivo.

Another aspect of the present invention relates to a method of delivering a nucleic acid to a cell, comprising the step of:

contacting a cell with an effective amount of a mixture comprising a nucleic acid and a compound of formula I-X tethered to a surface.

In certain embodiments, the present invention relates to the aforementioned method, wherein said surface is mica, glass, polymer, metal, metal alloy, ceramic, or oxide.

In certain embodiments, the present invention relates to the aforementioned method, wherein said surface is mica.

In certain embodiments, the present invention relates to the aforementioned method, wherein said nucleic acid is DNA, RNA, plasmid, siRNA, duplex oligonucleotide, single-strand oligonucleotide, triplex oligonucleotide, PNA, or mRNA.

In certain embodiments, the present invention relates to the aforementioned method, wherein said nucleic acid consists of about 10 to about 5000 nucleotides.

In certain embodiments, the present invention relates to the aforementioned method, wherein said nucleic acid is DNA or RNA.

In certain embodiments, the present invention relates to the aforementioned method, wherein said nucleic acid is a DNA or RNA sequence related to a mammalian disease.

In certain embodiments, the present invention relates to the aforementioned method, wherein said nucleic acid is DNA.

In certain embodiments, the present invention relates to the aforementioned method, wherein said nucleic acid is a DNA or RNA sequence targeting a gene selected from the group consisting of retroviral gene, viral gene, drug resistance gene, oncogene, gene related to inflammatory response, cellular adhesion gene, hormone gene, and abnormally overexpressed genes involved in gene regulation.

In certain embodiments, the present invention relates to the aforementioned method, wherein said nucleic acid is a DNA or RNA sequence related to cancer, viral infection, bacterial infection, lysosomal storage disorder, hypertension, ischaemic disorder, or HIV.

In certain embodiments, the present invention relates to the aforementioned method, wherein said nucleic acid is a DNA sequence encoding a genetic marker selected from the group consisting of luciferase gene, beta-galactosidase gene, hygromycin resistance, neomycin resistance, and chloramphenicol acetyl transferase.

In certain embodiments, the present invention relates to the aforementioned method, wherein said nucleic acid is a DNA sequence encoding a protein selected from the group consisting of low density lipoprotein receptors, coagulation factors, gene suppressors of tumors, major histocompatibility proteins, antioncogenes, p16, p53, thymidine kinase, IL2, IL 4, and TNFa.

In certain embodiments, the present invention relates to the aforementioned method, wherein said nucleic acid is a DNA sequence encoding a viral antigen.

In certain embodiments, the present invention relates to the aforementioned method, wherein said nucleic acid is a DNA sequence encoding an RNA selected from the group consisting of sense RNA, antisense RNA, and ribozyme.

In certain embodiments, the present invention relates to the aforementioned method, wherein said nucleic acid is a DNA sequence encoding a lectin, mannose receptor, sialoadhesin, or retroviral transactivating factor.

In certain embodiments, the present invention relates to the aforementioned method, wherein said cell is an animal cell or plant cell.

In certain embodiments, the present invention relates to the aforementioned method, wherein said cell is a mammalian cell.

In certain embodiments, the present invention relates to the aforementioned method, wherein said cell is a human cell or insect cell.

In certain embodiments, the present invention relates to the aforementioned method, wherein said cell is a human cell.

In certain embodiments, the present invention relates to the aforementioned method, wherein said cell is an embryonic cell or stem cell.

In another aspect the present invention also relates to a process for transfecting a polynucleotide into cells wherein said process comprises contacting said cells with a composition prepared according to the use of the invention before, simultaneously or after contacting them with the polynucleotide. This process may be applied by direct administration of said composition to cells of the animal in vivo.

### Overview of Gene Therapy

Gene therapy has generally been conceived as principally applicable to heritable deficiency diseases (cystic fibrosis, dystrophies, haemophilias, etc.) where permanent cure may be effected by introducing a functional gene. However, a much larger group of diseases, notably acquired diseases (cancer, AIDS, multiple sclerosis, etc.) might be treatable by transiently engineering host cells to produce beneficial proteins.

Applications are, for example, the treatment of muscular dystrophies or of cystic fibrosis. The genes of Duchenne/Becker muscular dystrophy and cystic fibrosis have been identified and encode polypeptides termed dystrophin and cystic fibrosis transmembrane conductance regulator (CFTR), respectively. Direct expression of these genes within, respectively, the muscle or lung cells of patients should contribute to a significant amelioration of the symptoms by expression of the functional polypeptide in targeted tissues. Moreover, in cystic fibrosis studies have suggested that one would need to achieve expression of the CFTR gene product in only about 5% of lung epithelial cells in order to significantly improve the pulmonary symptoms.

Application in the area of treating hyperprolifertive disease include therapeutic genes coding for a protein selected from the following group of proteins: cytosine deaminase (CD), herpes simplex-virus thymidine kinase (HSV-TK), DNA-binding domain (DBD) of poly(ADP-ribose) polymerase (PARP), cytotoxic protease 2A and 3C of picornaviruses, preferably of enteroviruses, more preferably of group B Coxsackie viruses (CVB), in particular serotype B3. Cytosine deaminase converts 5-fluorocytosine to 5-fluorouracil which is incorporated into the DNA of replicating cells and then kills these cells. A systemic 5-fluorocytosine treatment in connection with local radiotherapy leads to a specific increase in the destruction of tumours, since cytosine deaminase is only formed in the tumour cells so that the dreaded side effects such as necroses/fibroses in neighbouring tissue, damage of bone marrow and intestinal mucosa, etc. are avoided. HSV-TK acts in a similar way; this enzyme activates gancyclovir which likewise incorporates into the DNA of replicating cells and destroys the DNA so that, in connection with local radiotherapy, the same advantages as with CD are attained. In contrast to CD and HSV-TK, expression of DBD molecules leads to inhibition of the activity of PARP which is required for repairing DNA damage. In this way it is not possible to “repair” again tumour cells “predamaged” in connection with the local radiotherapy, so that they die. In contrast, the proteases 2A and 3C induce apoptosis in cells and are thus cytotoxic.

Another application of gene therapy is vaccination. In this regard, the immunogenic product encoded by the polynucleotide introduced in cells of a vertebrate may be expressed and secreted or be presented by said cells in the context of the major histocompatibility antigens, thereby eliciting an immune response against the expressed immunogen. Functional polynucleotides can be introduced into cells by a variety of techniques resulting in either transient expression of the gene of interest, referred to as transient transfection, or permanent transformation of the host cells resulting from incorporation of the polynucleotide into the host genome. Successful gene therapy depends on the efficient delivery to and expression of genetic information within the cells of a living organism. Most delivery mechanisms used to date involve viral vectors, especially adeno- and retroviral vectors. Viruses have developed diverse and highly sophisticated mechanisms to achieve this goal including crossing of the cellular membrane, escape from lysosomal degradation, delivery of their genome to the nucleus and, consequently, have been used in many gene delivery applications in vaccination or gene therapy applied to humans. The use of viruses suffers from a number of disadvantages: retroviral vectors cannot accommodate large-sized DNA (for example, the dystrophin gene which is around 13 Kb), the retroviral genome is integrated into host cell DNA and may thus cause genetic changes in the recipient cell and infectious viral particles could disseminate in the organism or in the environment and adenoviral vectors can induce a strong immune response in treated patients (Mc Coy et al., Human Gene Therapy 6 (1995), 1553-1560; Yang et al., Immunity 1 (1996), 433-442).

Non-viral delivery systems have been developed which are based on receptor-mediated mechanisms (Perales et al., Eur. J. Biochem. 226 (1994), 255-266; Wagner et al., Advanced Drug Delivery Reviews 14 (1994), 113-135), on polymer-mediated transfection such as polyamidoamine (Haensler and Szoka, Bioconjugate Chem. 4 (1993), 372-379), dendritic polymer (WO 95/24221), polyethylene imine or polypropylene imine (WO 96/02655), polylysine (U.S. Pat. No. 5,595,897 or FR2 719 316) or on lipid-mediated transfection (Felgner et al., Nature 337 (1989), 387-388) such as DOTMA (Felgner et al., Proc. Natl. Acad. Sci. USA 84 (1987), 7413-7417), DOGS or Transfectam™ (Behr et al., Proc. Natl. Acad. Sci. USA 86 (1989), 6982-6986), DMRIE or DORIE (Felgner et al., Methods 5 (1993), 67-75), DC-CHOL (Gao and Huang, BBRC 179 (1991), 280-285), DOTAP™ (McLachlan et al., Gene Therapy 2 (1995), 674-622) or Lipofectamine™. These systems present potential advantages with respect to large-scale production, safety, targeting of transfectable cells, low immunogenicity and the capacity to deliver large fragments of DNA. Nevertheless their efficiency in vivo is still limited.

Therefore, one of the technical problems underlying the present invention is the provision of improved methods and means for the delivery of nucleic acid molecules in gene therapy. This particular technical problem is solved by the provision of the embodiments as defined in the claims.

In the scope of the present invention the term “transfection” means the transfer of the polynucleotide into a cell wherein the polynucleotide is not associated with viral particles. Thus, transfection is to be distinguished from infection which relates to polynucleotides associated with viral particles.

In a preferred embodiment the therapeutic composition prepared according to the use of the present invention is in a form for administration into a vertebrate tissue. These tissues include those of muscle, skin, brain, lung, liver, spleen, bone marrow, thymus, heart, lymph, bone, cartilage, pancreas, kidney, gall bladder, stomach, intestine, testis, ovary, uterus, rectum, nervous system, eye, gland, connective tissue, blood, tumor etc. Cells where the improved transfection of a foreign polynucleotide would be obtained are those found in each of the listed target tissues (muscular cells, airway cells, hematopoietic cells, etc.). The administration may be made by intradermal, subdermal, intravenous, intramuscular, intranasal, intracerebral, intratracheal, intraarterial, intraperitoneal, intravesical, intrapleural, intracoronary or intratumoral injection, with a syringe or other devices. Transdermal administration is also contemplated, as are inhalation or aerosol administration.

In certain embodiments, the therapeutic composition further comprises at least one polynucleotide. In a particularly preferred embodiment, the polynucleotide which is contained in the composition, contains and is capable of functionally expressing a gene in said cell. The polynucleotide may be a DNA or RNA, single or double stranded, linear or circular, natural or synthetic, modified or not (see U.S. Pat. No. 5,525,711, U.S. Pat. No. 4,711,955 or EP-A 302 175 for modification examples; all of which are incorporated by reference). It may be, inter alia, a genomic DNA, a cDNA, an mRNA, an antisense RNA, a ribosomal RNA, a ribozyme, a transfer RNA or DNA encoding such RNAs. “Polynucleotides” and “nucleic acids” are synonyms with regard to the present invention. The polynucleotide may also be in the form of a plasmid or linear polynucleotide which contains at least one expressible sequence of nucleic acid that can generate a polypeptide, a ribozyme, an antisense RNA or another molecule of interest upon delivery to a cell. The polynucleotide can also be an oligonucleotide which is to be delivered to the cell, e.g., for antisense or ribozyme functions.

In a particularly preferred embodiment of the invention the polynucleotide is a naked polynucleotide (Wolff et al., Science 247 (1990), 1465-1468) or is a polynucleotide associated or complexed with a polypeptide, with the proviso that when said polypeptide is a viral polypeptide, then said polynucleotide combined with the viral polypeptide does not form infectious viral particles, or with a cationic compound or with any component which can participate in the protection and uptake of the polynucleotide into the cells (see Ledley, Human Gene Therapy 6 (1995), 1129-1144 for a review). Cationic compounds to which the polynucleotide is complexed are preferably cationic lipids, especially those disclosed in WO 98/34910 (incorporated by reference). Both DNA or RNA can be delivered to cells to form therein a polypeptide of interest. In certain embodiments, the polynucleotide present in the therapeutic composition is in the form of plasmid DNA. If the polynucleotide contains the proper genetic information, it will direct the synthesis of relatively large amounts of the encoded polypeptide. When the polynucleotide delivered to the cells encodes an immunizing polypeptide, the use according to the invention can be applied to achieve improved and effective immunity against infectious agents, including intracellular viruses, and also against tumor cells. The genetic informations necessary for expression by a target cell comprise all the elements required for transcription of said DNA into mRNA and for translation of mRNA into polypeptide.

Transcriptional promoters suitable for use in various vertebrate systems are well known. For example, suitable promoters include viral promoters like RSV, MPSV, SV40, CMV or 7.5 k, vaccinia promoter, inducible promoters, etc. The polynucleotide can also include intron sequences, targeting sequences, transport sequences, sequences involved in replication or integration. Said sequences have been reported in the literature and can be readily obtained by those skilled in the art. The polynucleotide can also be modified in order to be stabilized with specific components as spermine.

In general, the concentration of the polynucleotide in the composition is from about 0.1 microg/ml to about 20 mg/ml. According to the invention, the polynucleotide can be homologous or heterologous to the target cells into which it is introduced. Advantageously said polynucleotide encodes all or part of a polypeptide, especially a therapeutic or prophylactic polypeptide. A polypeptide is understood to be any translational product of a polynucleotide regardless of size, and whether glycosylated or not, and includes peptides and proteins. Therapeutic polypeptides include as a primary example those polypeptides that can compensate for defective or deficient proteins in an animal or human organism, or those that act through toxic effects to limit or remove harmful cells from the body. They can also be immunity conferring polypeptides which act as endogenous immunogens to provoke a humoral or cellular response, or both.

It can also be advantageous for the described gene therapy if the part of the nucleic acid which codes for the polypeptide comprises one or more non-coding sequences including intron sequences, preferably between promoter and the polypeptide start codon, and/or a polyA sequence, in particular the naturally occurring polyA sequence or an SV40 virus polyA sequence, especially at the 3′ end of the gene, because this can achieve stabilization of the mRNA in the cell (Jackson, R. J. (1993) Cell, 74, 9-14 and Palmiter, R. D. et al. (1991) Proc. Natl. Acad. Sci. USA, 88, 478-482).

Examples of polypeptides encoded by the polynucleotide are enzymes, hormones, cytokines, membrane receptors, structural polypeptides, transport polypeptides, adhesines, ligands, transcription factors, traduction factors; replication factors, stabilization factors, antibodies, more especially CFTR, dystrophin, factors VIII or IX, E6 or E7 from HPV, MUC1, BRCA1, interferons, interleukin (IL-2, IL-4, IL-6, IL-7, IL-12, GM-CSF (Granulocyte Macrophage Colony Stimulating Factor), the tk gene from Herpes Simplex type 1 virus (HSV-1), p53, HGF or VEGF. The polynucleotide can also code for an antibody. In this regard, antibody encompasses whole immunoglobulins of any class, chimeric antibodies and hybrid antibodies with dual or multiple antigen or epitope specificities, and fragments, such as F(ab).sub.2, Fab′, Fab including hybrid fragments and anti-idiotypes (U.S. Pat. No. 4,699,880).

In a further preferred embodiment the composition further comprises at least one component selected from the group consisting of chloroquine, protic compounds such as propylene glycol, polyethylene glycol, glycerol, ethanol, 1-methyl L-2-pyrrolidone or derivatives thereof, aprotic compounds such as dimethylsulfoxide (DMSO), diethylsulfoxide, di-n-propylsulfoxide, dimethylsulfone, sulfolane, dimethyl-formamide, dimethylacetamide, tetramethylurea, acetonitrile or derivatives. Said composition can also comprises at least one component selected from the group consisting of cytokines, especially interleukin-10 (IL-10), and nuclease inhibitors such as, for example, actin G.

In another preferred embodiment the composition prepared according to the use of the invention can be used in a method for the therapeutic treatment of humans or animals. In this particular case, the composition may also comprise a pharmaceutically acceptable injectable carrier (for examples, see Remington's Pharmaceutical Sciences, 16.sup.th ed. 1980, Mack Publishing Co). The carrier is preferably isotonic, hypotonic or weakly hypertonic and has a relatively low ionic strength, such as provided by a sucrose solution.

Furthermore, it may contain any relevant solvents, aqueous or partly aqueous liquid carriers comprising sterile, pyrogen-free water, dispersion media, coatings, and equivalents, or diluents (e.g; Tris-HCl, acetate, phosphate), emulsifiers, solubilizers or adjuvants. The pH of the pharmaceutical preparation is suitably adjusted and buffered in order to be useful in in vivo applications.

Examples of nucleic acids which code for a therapeutically effective gene product are the nitric-oxide synthase gene, especially a gene which codes for inducible nitric-oxide synthase (see, for example, DE 44 11 402 A1), the erythropoietin gene (see, for example, EP 0 148 605 B1), the insulin gene (see, for example, EP 0 001 929 B1) or the genes coding for blood coagulation factors, interferons, cytokines, hormones, growth factors etc. Certain genes are those coding for proteins which occur in blood.

The somatic gene therapy according to the invention can eliminate or alleviate in a particularly simple and lasting manner for example a pathological deficiency phenomenon such as, for example, a deficiency of insulin in diabetics, a deficiency of factor VIII in haemophiliacs, a deficiency of erythropoietin in kidney patients, a deficiency of thrombopoietin or a deficiency of somatostatin associated with stunted growth, by increasing the plasma concentrations of the particular active substance. The present invention also encompasses therapy of vascular disorders, such as arteriosclerosis, stenosis or restenosis.

Cerebrovascular disorders can be treated or prevented by gene therapy with the HGF gene or VEGF gene. For example, it has been demonstrated that: (a) after the transfection of HGF gene or VEGF gene, these proteins are detected in the brain over a prolonged period of time; (b) by treatment using HGF gene or VEGF gene transfection, angiogenesis can be induced on the surface of an ischemic brain; (c) the transfection of HGF gene or VEGF gene is effective in treating reduced blood flow in the brain caused by obstruction in the blood vessels; and (d) this treatment method is also effective when performed before obstruction. Thus, HGF gene and VEGF gene may be effectively used as a therapeutic or preventive agent for various cerebrovascular disorders, such as disorders resulting from cerebral ischemia, disorders associated with reduced blood flow in the brain, disorders for which improvement is expected by promoting angiogenesis in the brain, and the like. Gene therapy with HGF and VGEF genes may be used as therapeutic or preventive agents for cerebrovascular obstruction, cerebral infarction, cerebral thrombosis, cerebral embolism, stroke (including subarachnoid bleeding, transient cerebral ischemia, cerebral atheroscrelosis), cerebral bleeding, moyamoya disease, cerebrovascular dementia, Alzheimer's dementia, sequelae of cerebral bleeding or cerebral infarction, and the like. Moreover, since HGF gene has c-Met-mediated nerve cell protecting effect, it can be effectively used as a therapeutic or preventive agent for neurodegenerative diseases such as Alzheimer's disease, Alzheimer's senile dementia, amyotrophic lateral sclerosis, or Parkinson's disease.

### Charge-Reversible Phospholipids for Gene Delivery

The delivery of nucleic acid to a cell offers the potential to correct a defective gene or introduce a new gene for a specific biological activity. As such, in vitro gene delivery is widely used in research laboratories and in vivo gene therapy holds promise for the cure of hereditary and environmentally induced genetic diseases including cancer. The current delivery approaches in use include, for example, viral vectors, synthetic cationic vectors, CaP particles, surface-mediated vectors, and electroporation. Of these, synthetic cationic vectors offer the advantages of low or minimal toxicity, nonimmunogenicity, ease of synthesis, and large nucleic acid payloads; but suffer from low transfection activities. This low activity likely reflects inefficiencies in the overall transfection pathway that includes DNA-synthetic vector complexation, endocytosis, endosomal escape, nuclear entry, and finally expression. Today, many synthetic cationic vectors such as 1,2-dioleoyloxy-3-(trimethylammonio)-propane (DOTAP) are used in conjunction with “helper” phospholipids, which allow fusion of the bilayer with the membrance of the endosome, to increase the transfection efficacy. These helper lipids are typically zwitterionic lipids such as dioleylphosphatidyl ethanolamine (DOPE) or dioleylphosphatidyl choline (DOPC).

An electrostatic transition intracellularly from a cationic amphiphile to an anionic amphiphile was postulated to be useful as a charge-reversal mechanism for delivery of a nucleic acid payload. To determine if this charge-reversal concept translates to a more efficient helper phospholipid, we prepared a zwitterionic charge-reversal phospholipid (Figure A). Herein, we describe the synthesis and characterization of a functional helper phospholipid that can undergo a reaction to afford a negative charge on each of its hydrocarbon chains, and that shows enhanced gene transfection when used with DOTAP. It is envisaged that such a transformation destabilizes the endosome bilayer, thereby facilitating DNA delivery.

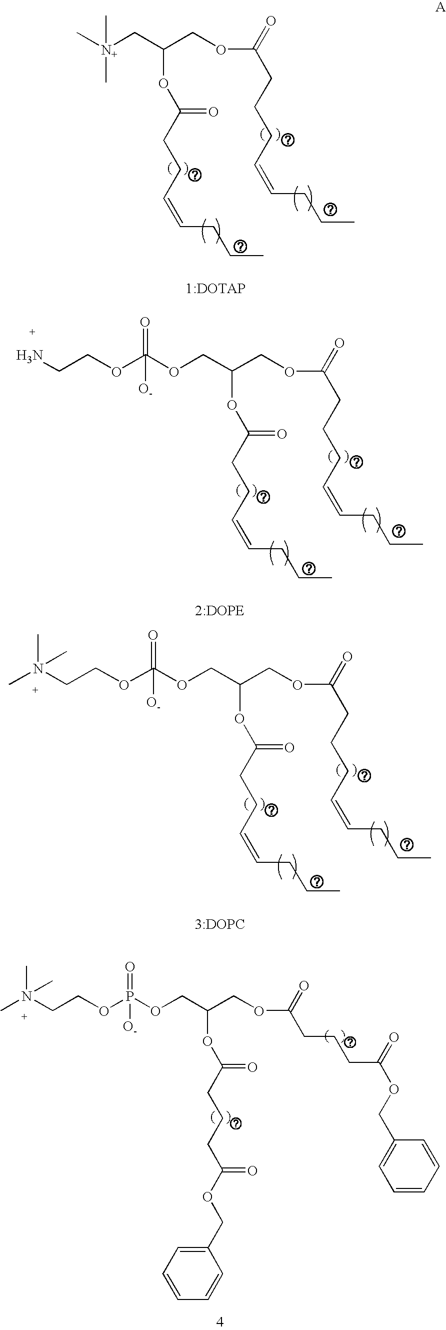

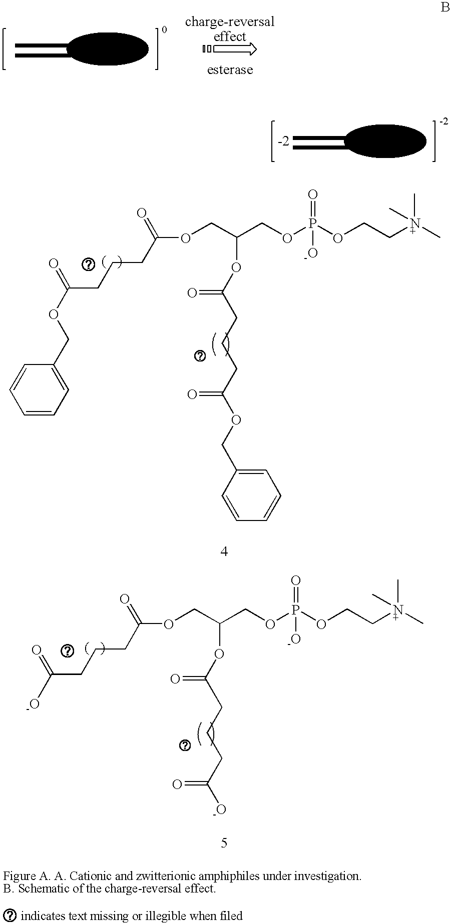

To test this approach, which exploits a change in electrostatic forces (0 to −2) to disrupt the DNA-vector supramolecular assembly, we prepared the zwitterionic charge-reversal phospholipid, 4 (Figure A). The lipid was synthesized as shown in Scheme 1. First, one equivalent of benzyl formate was added to an octane solution of dodecanoic diacid, in the presence of Dowex 50W-X2 to afford the mono benzyl-ester product. Next, the benzyl ester dodecanoic acid was coupled to glycerol-3-t-butyl-diphenyl silane in the presence of DCC and DMAP, in dichloromethane. The silyl group was removed with TBAF in THF. The reaction of chloro-oxo-dioxaphospholane with the deprotected compound was performed in THF at 0° C. in the presence of TEA. The resulting intermediate was transferred to a pressure tube and heated for one day with trimethylamine in acetonitrile and THF to give the desired product. Hydrogenloysis of 4 using Pd/C and H₂ afforded the di-anionic amphiphile 5.

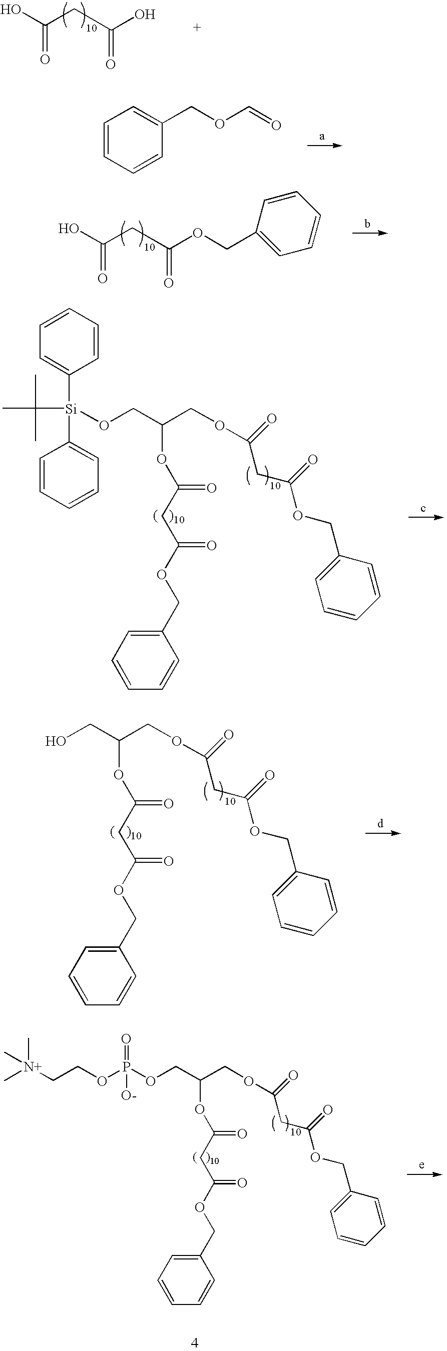

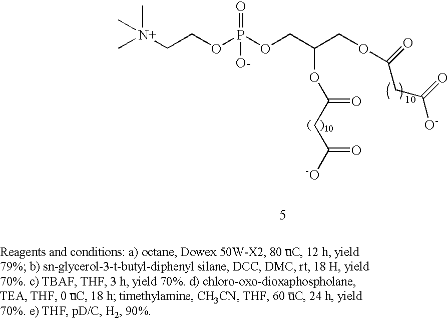

It is likely that 4 will form bilayers. A differential scanning calorimeter (DSC) trace of hydrated amphiphile 4 shows a phase-transition temperature at ˜44° C. The anionic lipid 5 does not exhibit a phase-transition temperature. Next, we prepared vesicles of 4, 4/DOTAP, and DOPC/DOTAP. A chloroform solution containing 4, 4/DOTAP, or DOPC/DOTAP was added to a pear shaped flask and the solution was evaporated under vacuum leaving a thin film deposited onto the flask wall. One mL of Tris buffer (100 mM Tris, 100 mM NaCl, pH 7.4) was then added and the film was peeled off by vortexing. The milky aqueous suspension was extruded through a polycarbonate membrane (50 nm) using an Aventi polar lipids mini-extruder. After 20 extrusions, a homogeneous liposome solution was observed. The average diameter, determined by dynamic light scattering, of the liposomes prepared from 4, 4/DOTAP, and DOPC/DOTAP was 79, 231, and 80 nm, respectively. Upon addition of an esterase to a solution of the liposomes prepared from 4, we detected complete hydrolysis of 4 to yield 5 in 8 hours by HPLC. Transmission electron micrographs (TEM) of 4 and 4/DOTAP showed similar results with vesicular organizations in both samples with an average size of about 100 nM. The structure of the vesicles formed by 4 was investigated at 25° C. by X-ray diffraction (SAXS). The diffraction patterns of the oriented multilayers of the hydrated vesicle pellet of 4 show a lamellar structure with a similar d spacing of 6.1 nm. In the presence of DNA, the 4/DOTAP/DNA assembly d spacing increases to 7.6 nm. This 1.5 nm increase in repeat period is similar to that observed for other bilayers containing cationic lipids when DNA is incorporated between adjacent bilayers.

We were unable to obtain patterns from pellets of 4/DOTAP. Upon addition of an esterase to a solution of the liposomes prepared from 4, we detected loss of 4 in four hours by HPLC analysis (see SI). The anionic lipid 5, which has a net charge of −2 (compared to the 0 net charge of 4) formed through hydrolysis of the terminal ester linkages, does not exhibit a phase-transition temperature. The anionic lipid, 5, can destabilize bilayers as evident from DSC doping studies with DPPC. The DPPC phase-transition broadens with increasing added amounts of 5 (see SI). We propose that a role of this functional helper phospholipid is to destabilize bilayers through formation of negative charges in the hydrocarbon chains.

The propensity of the lipids to bind DNA was measured via an ethidium bromide displacement fluorescence assay. This assay entailed measuring the reduction of the fluorescence intensity of the DNA-intercalated ethidium bromide, as this fluorescent probe is displaced by the cationic amphiphile. Figure B shows the fluorescence intensity as a function of vector/DNA charge ratio. The fluorescence intensity decreases upon addition of DOTAP, 4/DOTAP, DOTAP/DOPE, and DOPC/DOTAP. The results obtained with DOTAP, DOTAP/DOPE, and DOPC/DOTAP are consistent with previous reports. A 1:1 assembly is formed between the lipids and DNA. The zwitterionic lipids 4, DOPC, and DOPE as well as the anionic lipid, 5 do not displace EtBr consistent with the unfavorable electrostatic interactions for binding with the anionic DNA.

Transfection experiments with 4, 4/DOTAP, DOPE/DOTAP and DOTAP using the reporter gene, β-galactosidase (β-gal, pVax-LacZ1, Invitrogen), were performed with Chinese hamster ovarian (CHO) cells and L6 cells. The DOPE/DOTAP system was used instead of DOPC/DOTAP since the former is found to be more active. The reporter gene was first mixed with the lipids in potassium phosphate buffer (PBS) at room temperature. We tested two different lipid/DNA ratios (5:1; and 20:1) while keeping the zwitterionic/DOTAP ratio constant at 1:1. Next, the lipid/DNA complexes were added to the cells. The amount of DNA used was the same as used in the naked DNA control (no lipid), and the negative control was compound 4 without DNA. After incubation at 37° C. and 5% CO₂ for 2 h, the medium was removed and fresh growth medium was added. Transfection efficiencies were assessed after 48 h using the β-galactosidase enzyme assay in conjunction with a standard curve. The efficiency of each transfection was calculated as β-gal activity normalized to total protein. The zwitterionic charge-reversible lipid by itself does not transfect DNA. As expected upon addition of DOPE to DOTAP, the transfection efficacy increased. In the presence of 4/DOTAP at a ratio of 1:1 (with amphiphile/DNA ratio of 20:1), the transfection increased ≈400% when compared with similar conditions to DOPE/DOTAP under the same conditions (Figure C). Increasing the 4:DOTAP ratio from 1:1 to 2:1 afforded higher activity but further increases in the ratio yielded less transfection. With these encouraging results, we evaluated the 4:DOTAP vector system for gene transfection in L6 cells. As shown in Figure C, the use of the zwitterionic charge-reversal amphiphile 4 and DOTAP increased the transfection level by about four-fold compared to DOPE/DOTAP.

Cytotocixity experiments were also performed with CHO and L6 cells using a formazan-based proliferation assay and a total protein assay. The cells were seeded onto a 96-multiwell plate with an appropriate density of 1×10⁴ cells per well. After 24 hours, 4, 4/DOTAP, DOTAP, and DOPE/DOTAP were added to the cells. After another 24 hours, cell proliferation/number was determined and expressed as a percentage of non-treated cells. None of the amphiphiles showed significant cytotoxicity, with results similar to the negative control (i.e., non treated cells).

Thus, we have synthesized and characterized a functional helper zwitterionic phospholipid for use in the delivery of nucleic acids into cells. This helper phospholipid, like the “charge-reversal amphiphile” we previously synthesized, changes net charge upon an enzyme catalyzed reaction and belongs to a class of functional synthetic vectors that respond to stimuli. When combined with DOTAP, this functional phospholipid affords a significant increase in gene delivery as measured by new protein expression in two different cell lines.

### DEFINITIONS

For convenience, certain terms employed in the specification, examples, and appended claims are collected here.

The term “nucleic acids” means any double strand or single strand deoxyribonucleic acid (DNA) or ribonucleic acid (RNA) of variable length. Nucleic acids include sense and anti-sense strands. Nucleic acid analogs such as phosphorothioates, phosphoramidates, phosphonates analogs are also considered nucleic acids as that terms is used herein. Peptide nucleic acids and other synthetic analogs of nucleic acids which have therapeutic value are also included. Nucleic acids also include chromosomes and chromosomal fragments.

The term “liposome” as used herein refers to a closed structure comprising of an outer lipid bi- or multi-layer membrane surrounding an internal aqueous space. Liposomes can be used to package any biologically active agent for delivery to cells. For example, DNA can be packaged into liposomes even in the case of plasmids or viral vectors of large size. Such liposome encapsulated DNA is ideally suited for use both in vitro, ex vivo, and in vivo. Liposomes generally from a bilayer membrane. These liposomes may form hexagonal structures, and suspension of multilamellar vesicles.

The term “transfection” describes the process by which foreign genes (“transgenes”) are introduced into a living host cell. Host cells that express or incorporate the foreign DNA are known as “transformed cells,” and the process by which they become transformed is called “transformation” or “transduction.” Different types of cells vary in their susceptibility to transformation, and protocols for introducing the foreign DNA are typically optimized.

The term “heteroatom” is art-recognized and refers to an atom of any element other than carbon or hydrogen. Illustrative heteroatoms include boron, nitrogen, oxygen, phosphorus, sulfur and selenium.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl”, “heteroaryl”, or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas:

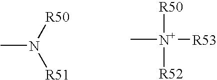

wherein R50, R51 and R52 each independently represent a hydrogen, an alkyl, an alkenyl, —(CH₂)ₙ-R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In other embodiments, R50 and R51 (and optionally R52) each independently represent a hydrogen, an alkyl, an alkenyl, or —(CH₂)ₘ-R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula:

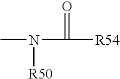

wherein R50 is as defined above, and R54 represents a hydrogen, an alkyl, an alkenyl or —(CH₂)ₘ-R61, where m and R61 are as defined above.

The term “amido” is art recognized as an amino-substituted carbonyl and includes a moiety that may be represented by the general formula:

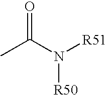

wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carboxyl” is art recognized and includes such moieties as may be represented by the general formulas:

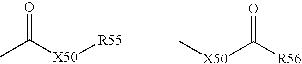

wherein X50 is a bond or represents an oxygen or a sulfur, and R55 and R56 represents a hydrogen, an alkyl, an alkenyl, —(CH₂)ₘ-R61 or a pharmaceutically acceptable salt, R56 represents a hydrogen, an alkyl, an alkenyl or —(CH₂)ₘ-R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The term “carbamoyl” refers to —O(C—O)NRR′, where R and R′ are independently H, aliphatic groups, aryl groups or heteroaryl groups.

The term “oxo” refers to a carbonyl oxygen (═O).

The terms “oxime” and “oxime ether” are art-recognized and refer to moieties that may be represented by the general formula:

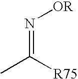

wherein R⁷⁵ is hydrogen, alkyl, cycloalkyl, alkenyl, alkynyl, aryl, aralkyl, or —(CH₂)ₘ-R61. The moiety is an “oxime” when R is H; and it is an “oxime ether” when R is alkyl, cycloalkyl, alkenyl, alkynyl, aryl, aralkyl, or —(CH₂)ₘ-R61.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ-R61, where m and R61 are described above.

The term “sulfonate” is art recognized and refers to a moiety that may be represented by the general formula:

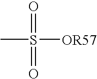

in which R57 is an electron pair, hydrogen, alkyl, cycloalkyl, or aryl.

The term “sulfate” is art recognized and includes a moiety that may be represented by the general formula:

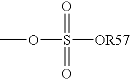

in which R57 is as defined above.

The term “sulfonamido” is art recognized and includes a moiety that may be represented by the general formula:

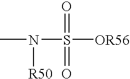

in which R50 and R56 are as defined above.

The term “sulfamoyl” is art-recognized and refers to a moiety that may be represented by the general formula:

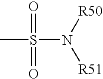

in which R50 and R51 are as defined above.

The term “sulfonyl” is art-recognized and refers to a moiety that may be represented by the general formula:

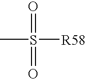

in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula:

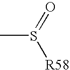

in which R58 is defined above.

The term “phosphoryl” is art-recognized and may in general be represented by the formula:

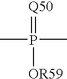

wherein Q50 represents S or O, and R59 represents hydrogen, a lower alkyl or an aryl. When used to substitute, e.g., an alkyl, the phosphoryl group of the phosphorylalkyl may be represented by the general formulas:

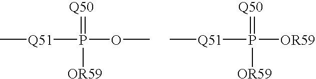

wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas:

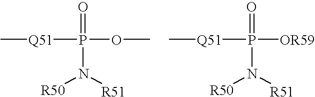

wherein Q51, R50, R51 and R59 are as defined above.

The term “phosphonamidite” is art-recognized and may be represented in the general formulas:

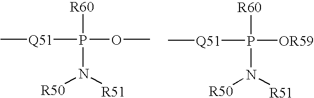

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ-R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

The term “alkali metal” refer to those elements listed in Group 1 of the periodic table. The following elements are alkali metals: Li, Na, K, Rb, Cs, and Fr.

For purposes of this invention, the chemical elements are identified in accordance with the Periodic Table of the Elements, CAS version, Handbook of Chemistry and Physics, 67th Ed., 1986-87, inside cover.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

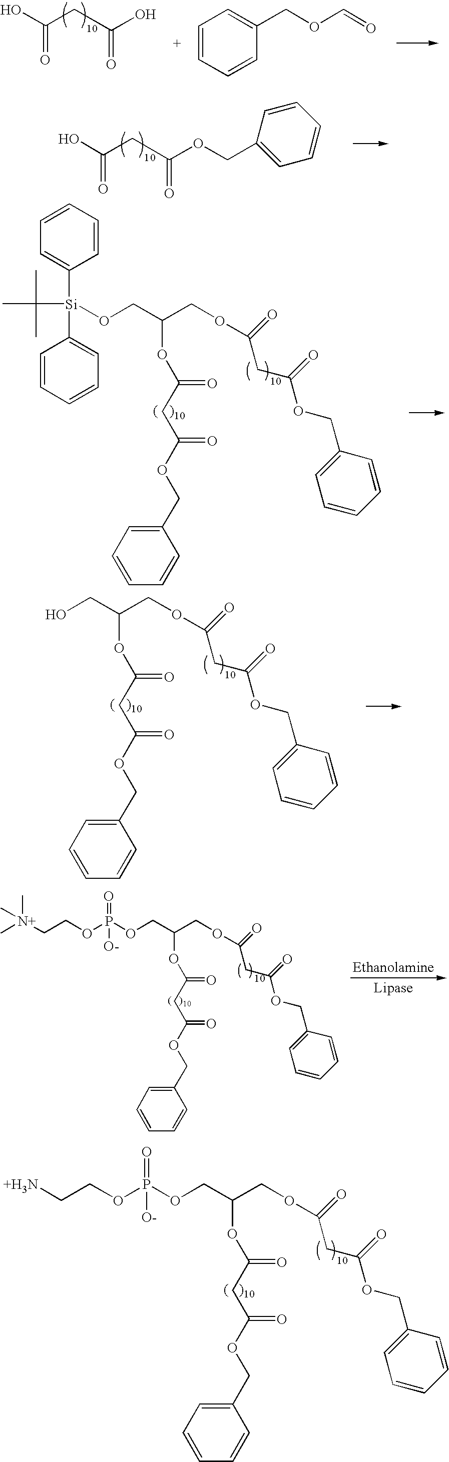

Dodecanedioic acid monobenzyl ester: Dodecanoic diacid (1 mmol) and Dowex 50W-X2 (50-100 mesh) (1.0 g) were stirred in benzyl formate/octane (2:8, 10 mL) at 80° C. The reaction was stirred for 12 h. The solution was then filtered and the filtrate evaporated. The crude product was purified by column chromatography (Hexane/EtOAc 8:2) to afford the compound as a white powder.

3-tert-butyldiphenyl silyl-sn-glycerol: Glycerol (1 mmol), tert-butyldiphenyl silane chloride (1 mmol) and imidazole (1 mmol) were dissolved in DMF. The reaction mixture was stirred for 2 days. The solution was filtered and the solvent removed under reduced pressure. The crude product was purified by column chromatography (Hexane/EtOAc 8:2) to afford the compound as a white powder.

1,2-Di-dodecanedioyl benzyl ester-3-tert-butyl diphenyl silyl-rac-glycerol: To a solution of dodecanoic acid benzyl ester (2.2 mmol), sn-glycero-3-tert-butyl diphenyl silane (1, mmol) and DMAP (catalytic amount) in DCM (20 mL) was added DCC (2.2 mmol). The solution stirred for 18 h and then it was filtered to remove the DCU precipitate. Concentration of the filtrate followed by chromatography (Hexane/EtOAc 9:1) afforded the product as white powder.

1,2-Di-dodecanedioyl benzyl ester-rac-glycerol: One mmol of 1,2-di-dodecanedioyl benzyl ester-3-tert-butyl diphenyl silyl-rac-glycerol was dissolved in 50 mL of THF. Tetrabutylammonium fluoride trihydrate (4 mmol) was added to the reaction and the mixture was stirred for 1 hour. After one hour the reaction was complete as indicated by TLC. The solution was diluted with 10 mL of H₂O and acidified with 1 N HCl to a pH of 3. The product was extracted into DCM, dried over Na₂SO₄, and evaporated to dryness. The residue was purified by chromatography (Hexane/EtOAc 9:2) to afford the product as colorless oil.

1,2-Di-dodecanedioyl benzyl ester-3-phosphocholine-rac-glycerol: A solution of 1,2-di-dodecanedioyl benzyl ester-rac-glycerol (0.97 mmol) and TEA (19 mmol) in THF was cooled to 0° C. and chloro-2-oxo-1,2,3-dioxaphosphonate (1.55 mmol) was added drop wise. The reaction mixture was stirred at room temperature for 18 h followed by the filtration of the TEA salts at 0° C. The solvent was evaporated and the residue was used in the next step without purification. Anhydrous trimethylamine was condensed at 0° C. under nitrogen in a pressure tube. Next, the solution of oxo-dioxaphospholane product was added. The reaction mixture was stirred at 60° C. for 3 days. Evaporation of the solvent and purification by reverse phase chromatography (acetonitrile/water) afforded the product.

1,2-Di-dodecanedioyl benzyl ester-3-phosphoethanolamine-rac-glycerol: Ethanolamine (1.58 mmol) was dissolved in 0.6 mL of 100 mM NaOAc and 50 mM CaCl2 buffer, acetic acid was added to adjust the pH to 6.5. Then, phospholipase D (LPD) (100 units in 100 μL of 100 mM NaOAc buffer) was added, and the solution was mixed with 1,2-di-dodecanedioyl benzyl ester-3-phosphocholine-rac-glycerol (0.26 mmol) in 5 mL of chloroform. The reaction mixtures was shaken at 30° C. for 4 h. The organic layer was then separated, washed twice with water, and evaporated. Purification by silica gel chromatography afforded the desired product.

### Example 2

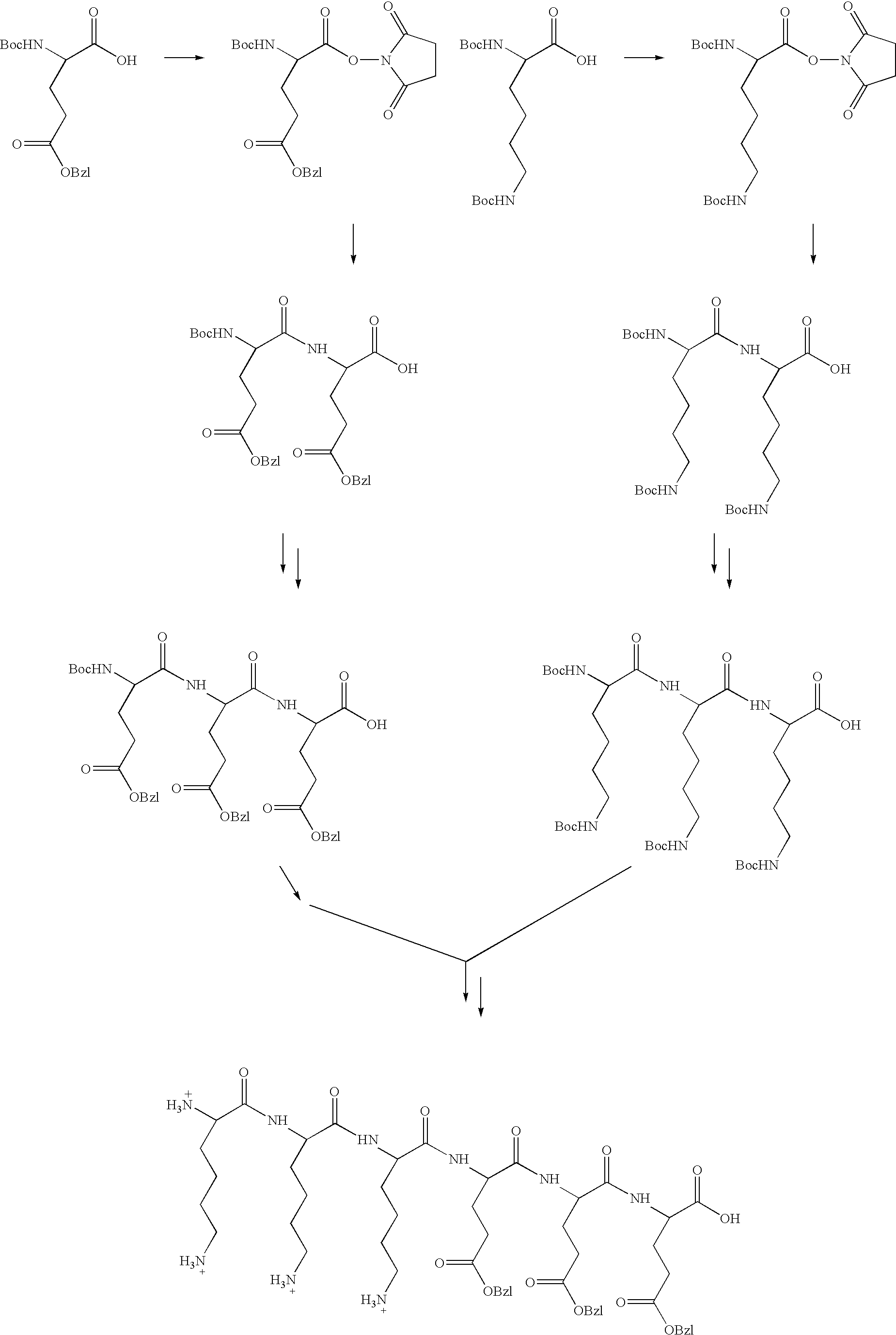

Boc-Glu(OBzl)-ONSu: To a solution of Boc-Glu(OBzl)-OH (1.26 mmol) and HONSu (1.39 mmol) in THF at −20° C., was added DCC (1.39 mmol). The mixture was stirred overnight at −20° C. The DCU was removed by filtration and the THF was removed by evaporation under vacuum. The crude compound was purified by recrystallization from ether.

Boc-Lys(boc)-ONSu: Same procedure used then that described for Boc-Glu(OBzl)-ONSu.

Boc-Glu(OBzl)-Glu(OBzl)-OH: To a solution of L-Glu(OBzl) (1.3 mmol), and NaHCO₃ (1.4 mmol) in water, was added a solution of Boc-Glu(OBzl)-ONSu in THF. The mixture was stirred overnight at room temperature. The solution was evaporated and acidified with 10% citric acid and extracted with ethyl acetate. The solution was washed with brine and dried over sodium sulfate. The crude product was purified by recrystallization from ether.

Boc-Lys(boc)-Lys(boc)-OH: Same procedure as described for Boc-Glu(OBzl)-Glu(OBzl)-OH.

Boc-Glu(OBzl)-Glu(OBzl)-ONSu: To a solution of Boc-Glu(OBzl)-Glu(OBzl)-OH (1.26 mmol) and HONSu (1.39 mmol) in THF at −20° C., was added DCC (1.39 mmol). The mixture was stirred overnight at −20° C. The DCU was removed by filtration and the THF was removed by evaporation under vacuum. The crude compound was purified by recrystallization from ether.

Boc-Lys(boc)-Lys(boc)-ONSu: Same procedure as described for Boc-Glu(OBzl)-Glu(OBzl)-ONSu.

Boc-Glu(OBzl)-Glu(OBzl)-Glu(OBzl)-OH: To a solution of L-Glu(OBzl) (1.3 mmol), and TEA (1.4 mmol) in THF, was added a solution of Boc-Glu(OBzl)-Glu(OBzl)-ONSu in THF. The mixture was stirred overnight at room temperature. The solution was evaporated and acidified with 10% citric acid and extracted with ethyl acetate. The solution was washed with brine and dried over sodium sulfate. The crude product was purified by recrystallization from ether.

Boc-Lys(boc)-Lys(boc)-Lys(boc)-OH: Same procedure as described for Boc-Glu(OBzl)-Glu(OBzl)-Glu(OBzl)-OH.

Glu(OBzl)-Glu(OBzl)-Glu(OBzl)-OH: Boc-Glu(OBzl)-Glu(OBzl)-Glu(OBzl)-OH was dissolved in a mixture 1:9 TFA/dichloromethane. After 2 h of stirring, the solvent was removed and the residue washed several times with ether. The powder was collected and dried under vacuum.

Boc-Lys(boc)-Lys(boc)-Lys(boc)-ONSu: Same procedure as described for Boc-Glu(OBzl)-Glu(OBzl)-ONSu.

Boc-Lys(boc)-Lys(boc)-Lys(boc)-Glu(OBzl)-Glu(OBzl)-Glu(OBzl)-OH: To a solution of Glu(OBzl)-Glu(OBzl)-Glu(OBzl)-OH (1.3 mmol), and TEA (1.4 mmol) in THF, was added a solution of Boc-Lys(boc)-Lys(boc)-Lys(boc)-ONSu in THF. The mixture was stirred overnight at room temperature. The solution was evaporated and acidified with 10% citric acid and extracted with ethyl acetate. The solution was washed with brine and dried over sodium sulfate. The crude product was purified by recrystallization from ether.

Lys-Lys-Lys-Glu(OBzl)-Glu(OBzl)-Glu(OBzl)-OH: Boc-Lys(boc)-Lys(boc)-Lys(boc)-Glu(OBzl)-Glu(OBzl)-Glu(OBzl)-OH was dissolved in a mixture 1:9 TFA/dichloromethane. After 2 h of stirring, the solvent was removed and the residue washed with ether several times. The powder was collected and dried under vacuum. Alternative synthesis of Compound 6:

N-ten-butoxycarbonyl-L-lysine-N-carboxyanhydride: To a suspension of Boc-Lys(boc)-OH (1.45 mmol) in ethyl acetate was added triphosgene (0.45 mmol). The suspension was vigorously stirred at room temperature. After 10 min, TEA (0.5 mmol) was added. Upon the addition of TEA, precipitation of TEA-HCl salt was observed. After stirring at room temperature for 5 h, the reaction mixture was cooled at −20° C. The solution was filtered and washed with ice water and 0.5% NaHCO₃. The organic phase was separated, dried over sodium sulfate, filtered and concentrated under reduced pressure. The addition of ether resulted in the precipitation of the compound.

Benzyl-L-glutamate-N-carboxyanhydride: A suspension of Glu(OBzl)-OH (1.43 mmol) in 50 mL of THF was heated at 50° C. under a nitrogen atmosphere. A solution of triphosgene (0.57 mmol) in 5 mL of THF was added dropwise to the reaction mixture.

When the reaction mixture started to become transparent, a stream of nitrogen was bubbled through the solution. After the reaction was complete the solvent was evaporated under reduce pressure to give an oily residue which crystallize upon cooling. The compound was obtained by recrystallization from ether.

Random Polymer:

N-tert-butoxycarbonyl-L-lysine-N-carboxyanhydride (15 eq) and benzyl-L-glutamate-N-carboxyanhydride (15 eq) were dissolved in DMF under N₂ atmosphere. Then, octylamine (1 eq) was added to the solution. The reaction mixture was stirred for 5 days and then precipitated in ether.

Block Polymer:

Boc-Lys-N-carboxyanhydride (15 eq) was dissolved in DMF under a nitrogen atmosphere. Then, octylamine (1 eq) was added to the solution. The reaction mixture was stirred for 5 days and then precipitated in ether. In a second step, the poly-Lys(boc)-NH₂, and benzyl-L-glutamate-N-carboxyanhydride (15 eq) was dissolved in DMF under N₂ atmosphere. The reaction mixture was stirred for 5 days and then precipitated in ether and dried under vacuum.

Deprotection of the Polymer:

The copolymer was dissolved in a mixture 5:5 TFA/dichloromethane. After 24 h of stirring, the solvent was removed and the residue washed with ether several times. The powder was collected and dried under vacuum.

### Example 3

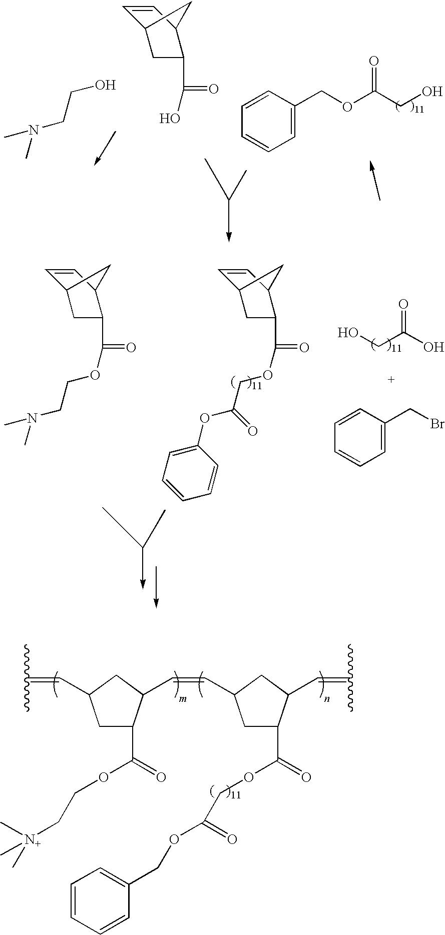

12-hydroxy-dodecaonoic acid benzyl ester: To a solution of 12-hydroxy-dodecanoic acid (4.62 mmol) in 7 mL DMF was slowly added at 0° C. benzyl bromide (6.43 mmol) and DBU (7.23 mmol). DMF was removed under reduced pressure. The residue was dissolve in dichloromethane and washed with 1 M HCl and NaHCO₃, dried over NaSO₄. A white powder was obtained after purification through silica gel column (EtOAc/hexanes 2:8). Benzyl ester/alkyl chain/bicyclic monomer: To a solution of endo/exo-bicyclo[2.2.1]hept-5-ene-2-carboxylic acid (3.62 mmol) and 12-hydroxy-dodecanoic acid benzyl ester (3.62 mmol) in 10 mL CH₂Cl₂ was added a solution of DCC (3.98 mmol) and DMAP (0.4 mmol) in 5 mL CH₂Cl₂ at 0° C. After stirring at room temperature overnight, the solution was filtrate and purified through silica gel column (EtOAc/hexanes 2:8). A colorless oil was obtained in 97% yield.

Ethanolamine/bicyclic monomer: To a solution of endo/exo-bicyclo[2.2.1]hept-5-ene-2-carboxylic acid (2.17 mmol) and N,N-dimethylethanolamine (2.17 mmol) were dissolved in 3 mL CH₂Cl₂, was slowly added a solution of DCC (2.39 mmol) and DMAP (0.22 mmol) in 2 mL at 0° C. After stirring at room temperature overnight, the solution was filtrated and purified through silica gel column (EtOAc/MeOH 9:1). A colorless oil was obtained.

Polymerization: The two monomers (0.24 mmol) were dissolved in 2 mL dichloroethane. To this solution was added 5 mmol % Grubbs I catalyst in 1 mL dichloroethane. After stirring at room temperature overnight, 3 mL ethyl vinyl ether was added to quench. All solvent was removed and the compound was obtained by recrystallization from MeOH.

Quaternization of amines: The polymer is dissolved in 2 mL DCM and CH₃I (2 mL) was added. After stirring at room temperature overnight, the solvents were removed under reduced pressure. The compound was obtained by recrystallization from ether.

### Example 4

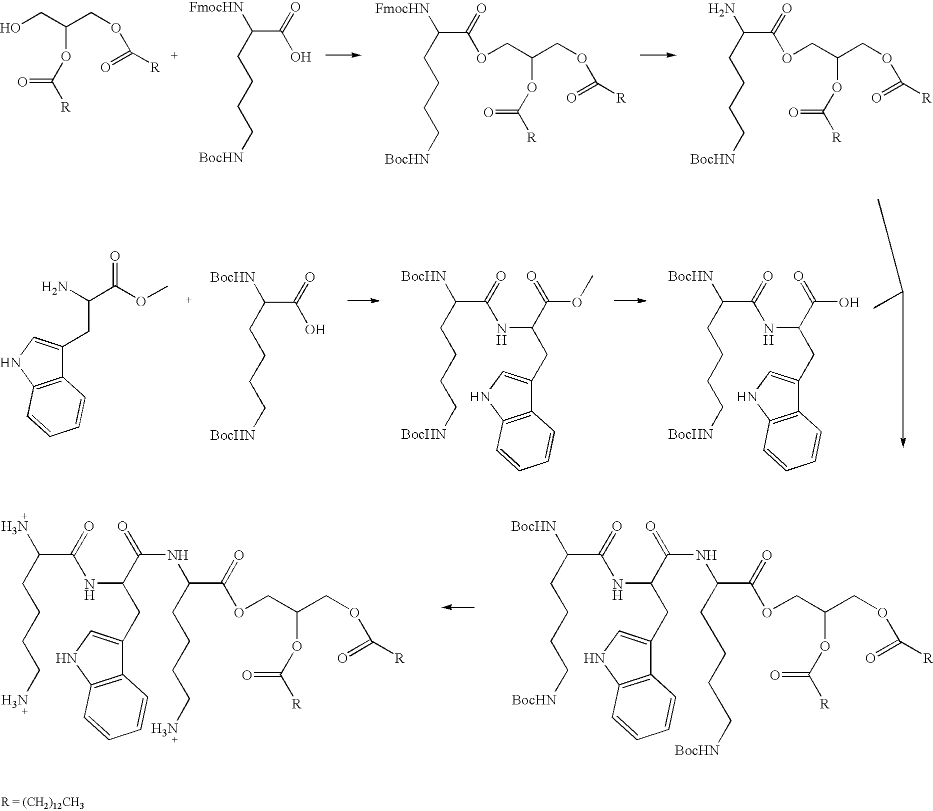

1,2-Di-tetradecanoyl-3-tert-butyl diphenyl silyl-rac-glycerol: Same procedure as described for 1,2-di-dodecanedioyl benzyl ester-3-tert-butyl diphenyl silyl-rac-glycerol.

1,2-Di-tetradecanoyl-rac-glycerol: Same procedure as described for 1,2-di-dodecanedioyl benzyl ester-rac-glycerol.

1,2-Di-tetradecanoyl-3-Fmoc-Lys(boc)-rac-glycerol: To solution of Fmoc-Lys(boc)-OH (1 mmol), 1,2-di-tetradecanoyl-rac-glycerol (1 mmol) and DMAP (catalytic amount) in DCM (20 mL) was added DCC (1.1 mmol). The solution was stirred for 18 h. The reaction mixture was then filtered to remove the insoluble DCU. Concentration of the filtrate followed by chromatography (Hexane/EtOAc 8:2) afforded the product as a white powder.

1,2-Di-tetradecanoyl-3-Lys(boc)-rac-glycerol: The 1,2-di-tetradecanoyl-Fmoc-Lys(boc)-rac-glycerol was dissolved in a solution of 5% of piperidine in DMF (3 mL). After stirring for 1 h the solvent was removed and the residue purified by chromatography (Hexane/EtOAc 8:2) to afford the product.

Boc-Lys(boc)-Trp-OMe: To a solution of Boc-lys(boc) (1 mmol), tryptophane methyl ester (1.1 mmol) and hydroxybenzotriazole (1 mmol) in DCM (20 mL) was added DCC (1.1 mmol). After the addition, the solution stirred for 18 h. The reaction mixture was then filtered to remove the insoluble DCU. Concentration of the filtrate followed by chromatography (Hexane/EtOAc 8:2) afforded the product as colorless oil.

Boc-Lys(boc)-Trp-OH: To a solution of Boc-Lys(boc)Trp-OMe in methanol was added a catalytic amount of NaOMe. The reaction was followed by TLC, after 1 h the reaction was complete and IRC 50 Dowex was added to the reaction mixture to neutralize the pH. After neutralization the solvent was removed to afford the compound.

1,2-Di-tetradecanoyl-3-Boc-Lys(boc)-Trp-Lys(boc)-rac-glycerol: To a solution of Boc-Lys(boc)-Trp-OH (1 mmol), 1,2-di-tetradecanoyl-Lys(boc)-rac-glycerol (1.1 mmol) and hydroxybenzotriazole (1 mmol) in DCM (20 mL) was added DCC (1.1 mmol). After the addition, the solution was stirred for 18 h. The reaction mixture was then filtered to remove the insoluble DCU. Concentration of the filtrate followed by chromatography (Hexane/EtOAc 8:2) afforded the product.

1,2-Di-tetradecanoyl-3-Lys-Trp-Lys-rac-glycerol: The 1,2-di-tetradecanoyl-3-Boc-Lys(boc)-Trp-Lys(boc)-rac-glycerol was dissolved in a mixture 1:9 TFA/dichloromethane. After 2 h of stirring, the solvent was removed and the residue was washed with ether. The product was dried under vacuum.

### Example 5

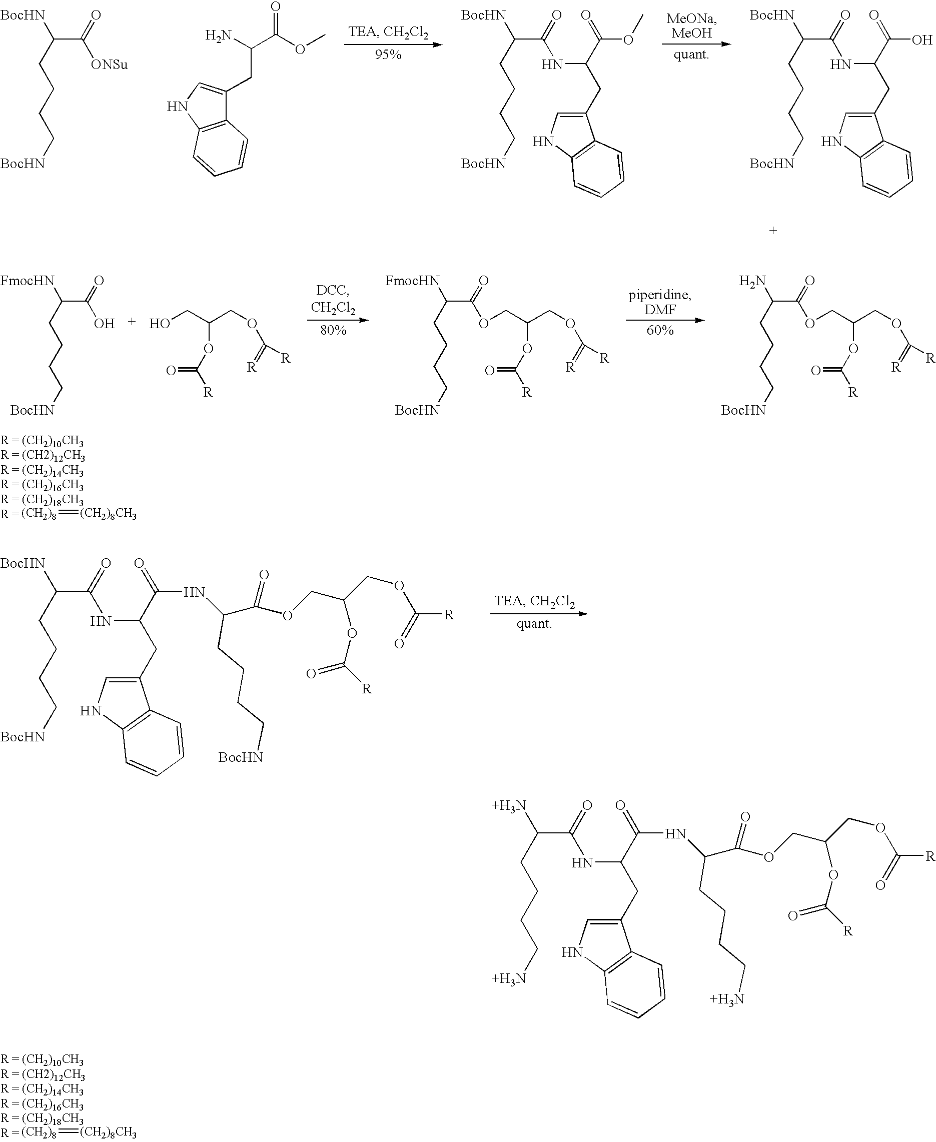

1,2-Di-dodecanoyl-3-tert-butyl diphenyl silyl-rac-glycerol: Same procedure used as that described for 1,2-di-dodecanedioyl benzyl ester-3-tert-butyl diphenyl silyl-rac-glycerol.

1,2-Di-tetradecanoyl-3-tert-butyl diphenyl silyl-rac-glycerol: Same procedure used as that described for 1,2-di-dodecanedioyl benzyl ester-3-tert-butyl diphenyl silyl-rac-glycerol.

1,2-Di-hexadecanoyl-3-tert-butyl diphenyl silyl-rac-glycerol: Same procedure used as that described for 1,2-di-dodecanedioyl benzyl ester-3-tert-butyl diphenyl silyl-rac-glycerol.

1,2-Di-octadecanoyl-3-tert-butyl diphenyl silyl-rac-glycerol: Same procedure used as that described for 1,2-di-dodecanedioyl benzyl ester-3-tert-butyl diphenyl silyl-rac-glycerol.

1,2-Di-oleyl-3-tert-butyl diphenyl silyl-rac-glycerol: Same procedure used as that described for 1,2-di-dodecanedioyl benzyl ester-3-tert-butyl diphenyl silyl-rac-glycerol.

1,2-Di-dodecanoyl-rac-glycerol: Same procedure used as that described for 1,2-di-dodecanedioyl benzyl ester-rac-glycerol.

1,2-Di-tetradecanoyl-rac-glycerol: Same procedure used as that described for 1,2-di-dodecanedioyl benzyl ester-rac-glycerol.

1,2-Di-hexadecanoyl-rac-glycerol: Same procedure used as that described for 1,2-di-dodecanedioyl benzyl ester-rac-glycerol.

1,2-Di-octadecanoyl-rac-glycerol: Same procedure used as that described for 1,2-di-dodecanedioyl benzyl ester-rac-glycerol.

1,2-Di-oleoyl-rac-glycerol: Same procedure used as that described for 1,2-di-dodecanedioyl benzyl ester-rac-glycerol.

Boc-Lys(boc)-Trp-OMe: To solution of tryptophane methyl ester (1.1 mmol) and TEA (2.2 mmol) in DCM (20 mL) was added Boc-Lys(boc)ONSu (1 mmol). After the addition, the solution stirred for 18 h. The reaction mixture was then evaporated and purified by chromatography (Hexane/EtOAc 8:2) afforded the product as colorless oil.

Boc-Lys(boc)-Trp-OH: To a solution of Boc-Lys(boc)Trp OMe in methanol was added a catalitic amount of NaOMe. The reaction was followed by tlc, after 1 h was done and a IRC 50 Dowex was added to the reaction mixture to neutralize the pH. After neutralization the solvent was removed to afford the compound.

1,2-Di-dodecanoyl-3-Fmoc-Lys(boc)-rac-glycerol: To solution of Fmoc-Lys(boc)-OH (1 mmol), 1,2-di-dodecanoyl-rac-glycerol (1 mmol) and DMAP (catalytic amount) in DCM (20 mL) was added DCC (1.1 mmol). After the addition, the solution stirred for 18 h. The reaction mixture was then filtered to remove the insoluble DCU. Concentration of the filtrate followed by chromatography (Hexane/EtOAc 8:2) afforded the product as white powder.

1,2-Di-tetradecanoyl-3-Fmoc-Lys(boc)-rac-glycerol: Same procedure used as that described for 1,2-Di-dodecanoyl-3-Fmoc-Lys(boc)-rac-glycerol.

1,2-Di-hexadecanoyl-3-Fmoc-Lys(boc)-rac-glycerol: Same procedure used as that described for 1,2-Di-dodecanoyl-3-Fmoc-Lys(boc)-rac-glycerol.

1,2-Di-octadecanoyl-3-Fmoc-Lys(boc)-rac-glycerol: Same procedure used as that described for 1,2-Di-dodecanoyl-3-Fmoc-Lys(boc)-rac-glycerol.

1,2-Di-oleoyl-3-Fmoc-Lys(boc)-rac-glycerol: Same procedure used as that described for 1,2-di-tetradecanoyl-3-Fmoc-Lys(boc)-rac-glycerol.

1,2-Di-dodecanoyl-3-Lys(boc)-rac-glycerol: The 1,2-di-dodecanoyl-Fmoc-Lys(boc)-rac-glycerol was dissolved in a solution of 5% of piperidine in DMF (3 mL). After stirring for 1 h the solvent was removed and the residue purified by chromatography (Hexane/EtOAc 8:2) afforded the product.

1,2-Di-tetradecanoyl-3-Lys(boc)-rac-glycerol: Same procedure used as that described for 1,2-di-dodecanoyl-3-Lys(boc)-rac-glycerol.

1,2-Di-hexadecanoyl-3-Lys(boc)-rac-glycerol: Same procedure used as that described for 1,2-di-dodecanoyl-3-Lys(boc)-rac-glycerol.

1,2-Di-octadecanoyl-3-Lys(boc)-rac-glycerol: Same procedure used as that described for 1,2-di-dodecanoyl-3-Lys(boc)-rac-glycerol.

1,2-Di-oleoyl-3-Lys(boc)-rac-glycerol: Same procedure used as that described for 1,2-di-dodecanoyl-3-Lys(boc)-rac-glycerol.

1,2-Di-dodecanoyl-3-Boc-Lys(boc)-Trp-Lys(boc)-rac-glycerol: To solution of Boc-Lys(boc)-Trp-OH (1 mmol), 1,2-di-dodecanoyl-3-Lys(boc)-rac-glycerol (1.1 mmol) and hydroxybenzotriazole (1 mmol) in DCM (20 mL) was added DCC (1.1 mmol). After the addition, the solution stirred for 18 h. The reaction mixture was then filtered to remove the insoluble DCU. Concentration of the filtrate followed by chromatography (Hexane/EtOAc 8:2) afforded the product.

1,2-Di-tetradecanoyl-3-Boc-Lys(boc)-Trp-Lys(boc)-rac-glycerol: Same procedure used as that described for 1,2-di-dodecanoyl-3-Boc-Lys(boc)-Trp-Lys(boc)-rac-glycerol.

1,2-Di-hexadecanoyl-3-Boc-Lys(boc)-Trp-Lys(boc)-rac-glycerol: Same procedure used as that described for 1,2-di-dodecanoyl-3-Boc-Lys(boc)-Trp-Lys(boc)-rac-glycerol.

1,2-Di-octadecanoyl-3-Boc-Lys(boc)-Trp-Lys(boc)-rac-glycerol: Same procedure used as that described for 1,2-di-dodecanoyl-3-Boc-Lys(boc)-Trp-Lys(boc)-rac-glycerol.

1,2-Di-oleoyl-3-Boc-Lys(boc)-Trp-Lys(boc)-rac-glycerol: Same procedure used as that described for 1,2-di-tetradecanoyl-3-Boc-Lys(boc)-Trp-Lys(boc)-rac-glycerol.

1,2-Di-dodecanoyl-3-Lys-Trp-Lys-rac-glycerol: The 1,2-di-dodecanoyl-3-Boc-Lys(boc)-Trp-Lys(boc)-rac-glycerol was dissolved in a mixture 1:9 TFA/dichloromethane. After 2 h stirring, the solvent was removed, the residue washed with ether several and dried under vacuum.

1,2-Di-tetradecanoyl-3-Lys-Trp-Lys-rac-glycerol: Same procedure used as that described for 1,2-di-dodecanoyl-3-Lys-Trp-Lys-rac-glycerol.

1,2-Di-hexadecanoyl-3-Lys-Trp-Lys-rac-glycerol: Same procedure used as that described for 1,2-di-dodecanoyl-3-Lys-Trp-Lys-rac-glycerol.

1,2-Di-octadecanoyl-3-Lys-Trp-Lys-rac-glycerol: Same procedure used as that described for 1,2-di-dodecanoyl-3-Lys-Trp-Lys-rac-glycerol.

1,2-Di-oleoyl-3-Lys-Trp-Lys-rac-glycerol: Same procedure used as that described for 1,2-di-dodecanoyl-3-Lys-Trp-Lys-rac-glycerol.

### Example 6

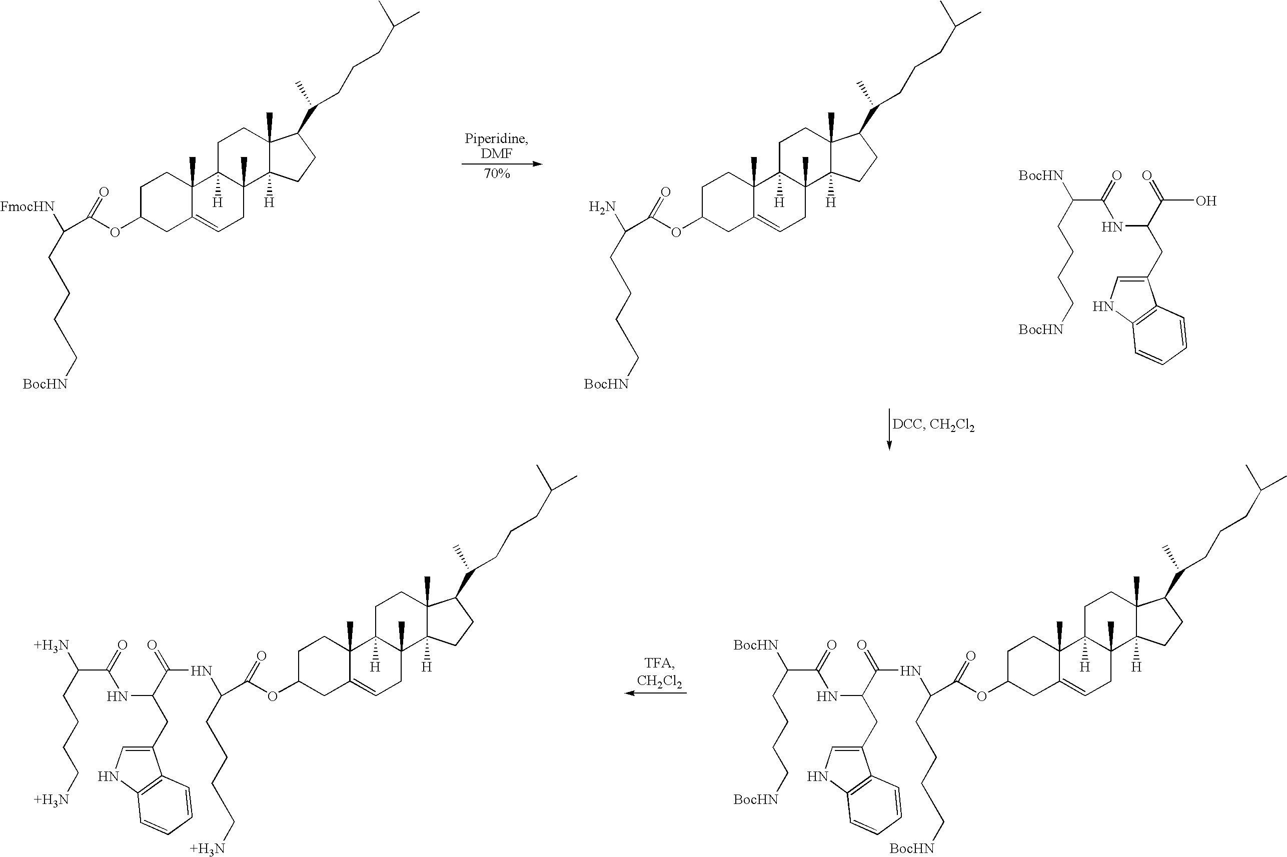

Fmoc-Lys(boc)-cholesterol: To solution of Fmoc-Lys(boc)-OH (1 mmol), cholesterol (1 mmol) and DMAP (catalytic amount) in DCM (20 mL) was added DCC (1.1 mmol). After the addition, the solution stirred for 18 h. The reaction mixture was then filtered to remove the insoluble DCU. Concentration of the filtrate followed by chromatography (Hexane/EtOAc 8:2) afforded the product as white powder.

Lys(boc)-cholesterol: Same procedure used as that described for 1,2-di-dodecanoyl-3-Lys(boc)-rac-glycerol.

Boc-Lys(boc)-Trp-Lys(boc)-cholesterol: Sane procedure used as that described for 1,2-di-dodecanoyl-3-Boc-Lys(boc)-Trp-Lys(boc)-rac-glycerol.

Lys-Trp-Lys-cholesterol: Same procedure used as that described for 1,2-di-dodecanoyl-3-Lys-Trp-Lys-rac-glycerol.

### Example 7

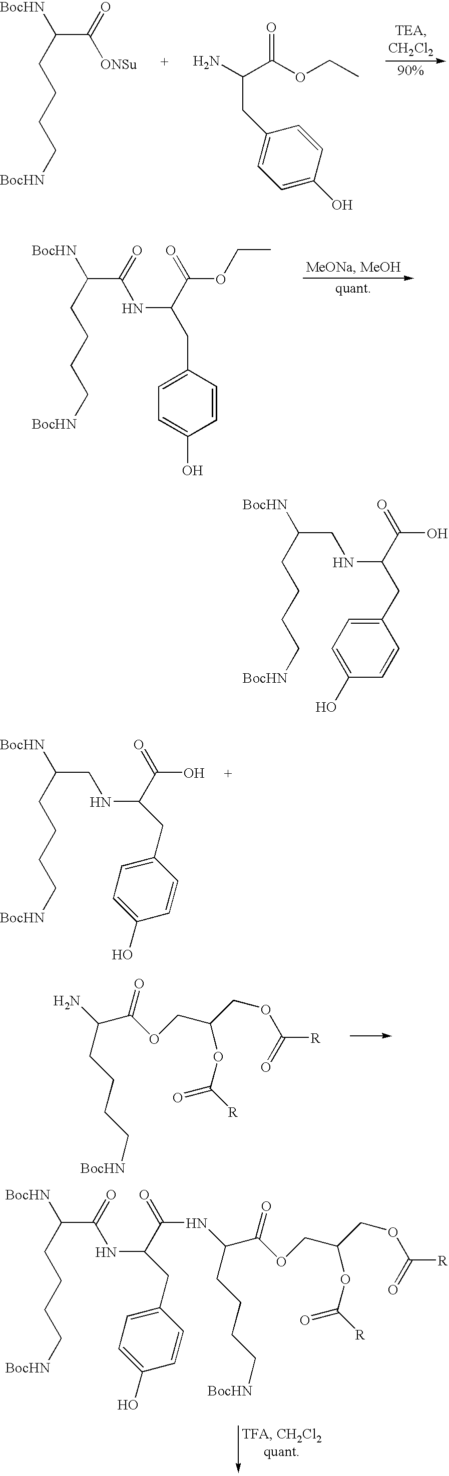

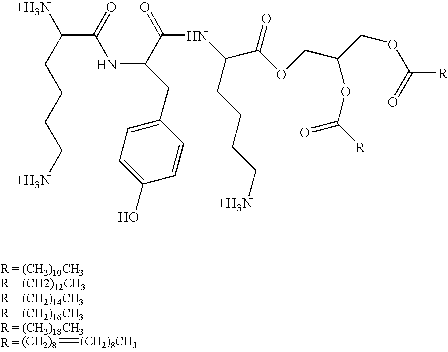

Boc-Lys(boc)-Tyr-OEt: Same procedure used then that described for Boc-Lys(boc)-Trp-OMe.

Boc-Lys(boc)-Tyr-OH: Same procedure used then that described for Boc-Lys(boc)-Trp-OH.

1,2-Di-tetradecanoyl-3-Boc-Lys(boc)-Tyr-Lys(boc)-rac-glycerol: Same procedure used then that described for 1,2-di-dodecanoyl-3-Boc-Lys(boc)-Trp-Lys(boc)-rac-glycerol.

1,2-Di-oleyl-3-Boc-Lys(boc)-Tyr-Lys(boc)-rac-glycerol: Same procedure used then that described for 1,2-di-dodecanoyl-3-Boc-Lys(boc)-Trp-Lys(boc)-rac-glycerol.

1,2-Di-tetradecanoyl-3-Lys-Tyr-Lys-rac-glycerol: Same procedure used then that described for 1,2-di-dodecanoyl-3-Lys-Trp-Lys-rac-glycerol.

1,2-Di-oleyl-3-Lys-Tyr-Lys-rac-glycerol: Same procedure used then that described for 1,2-di-dodecanoyl-3-Lys-Trp-Lys-rac-glycerol.

### Example 8

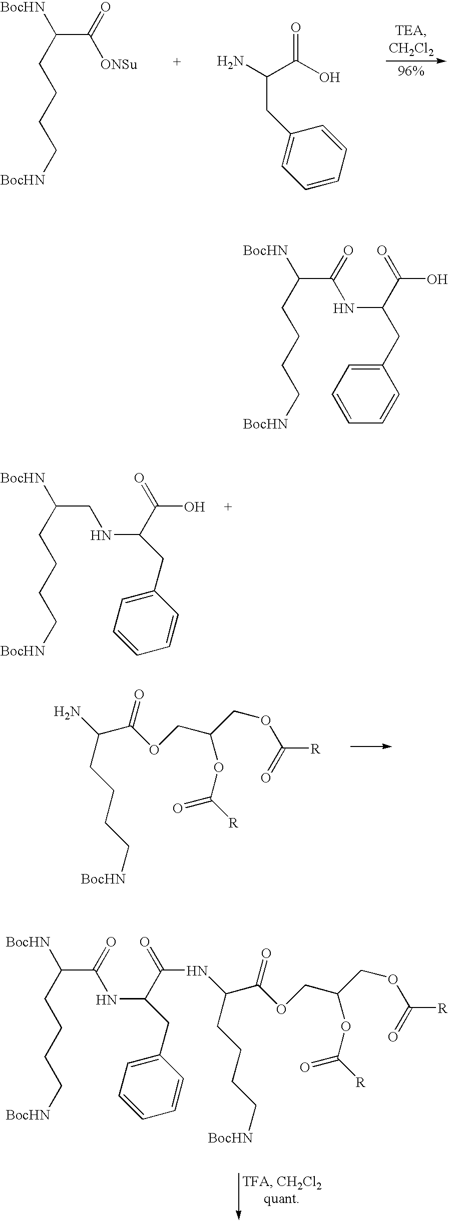

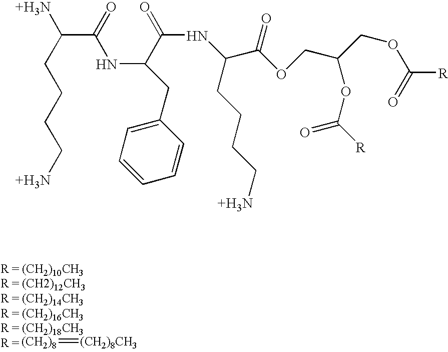

Boc-Lys(boc)-Phe-OH: Same procedure used then that described for Boc-Lys(boc)-Trp-OMe.

1,2-Di-tetradecanoyl-3-Boc-Lys(boc)-Phe-Lys(boc)-rac-glycerol: Same procedure used then that described for 1,2-di-dodecanoyl-3-Boc-Lys(boc)-Trp-Lys(boc)-rac-glycerol.

1,2-Di-oleyl-3-Boc-Lys(boc)-Phe-Lys(boc)-rac-glycerol: Same procedure used then that described for 1,2-di-dodecanoyl-3-Boc-Lys(boc)-Trp-Lys(boc)-rac-glycerol.

1,2-Di-tetradecanoyl-3-Lys-Phe-Lys-rac-glycerol: Same procedure used then that described for 1,2-di-dodecanoyl-3-Lys-Trp-Lys-rac-glycerol.

1,2-Di-oleyl-3-Lys-Phe-Lys-rac-glycerol: Same procedure used then that described for 1,2-di-dodecanoyl-3-Lys-Trp-Lys-rac-glycerol.

### Example 9

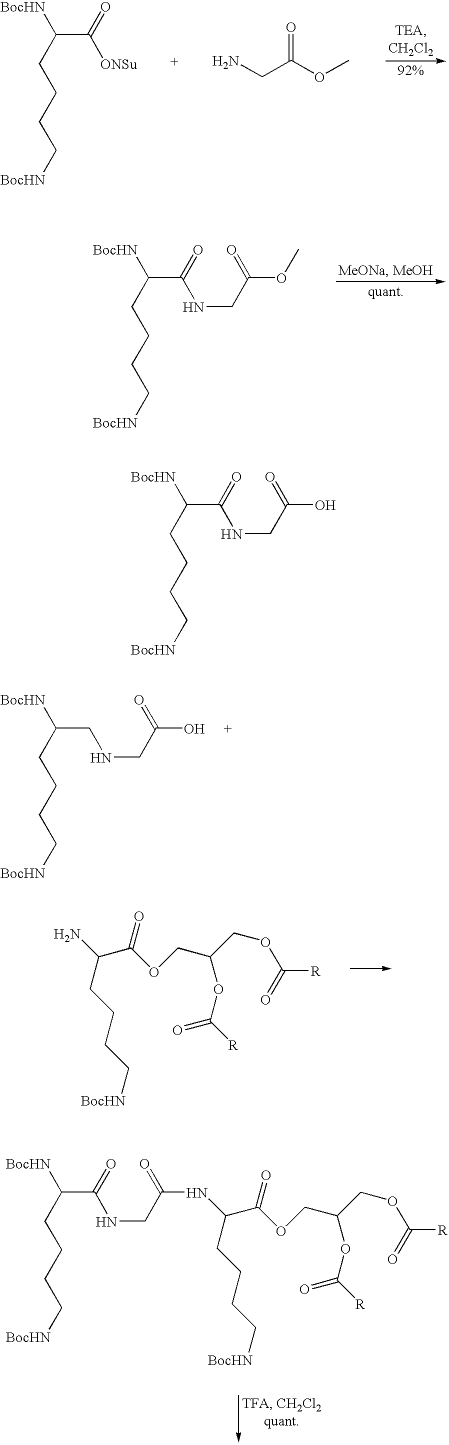

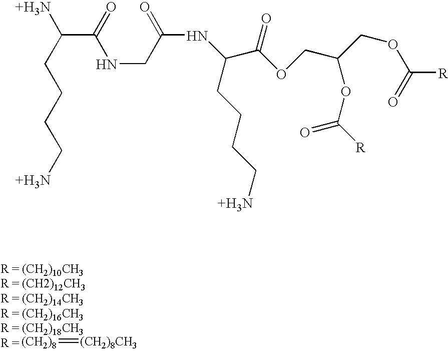

Boe-Lys(boc)-Gly-OMe: Same procedure used then that described for Boc-Lys(boc)-Trp-OMe.

Boc-Lys(boc)-Gly-OH: Same procedure used then that described for Boc-Lys(boc)-Trp-OH.

1,2-Di-tetradecanoyl-3-Boc-Lys(boc)-Gly-Lys(boc)-rac-glycerol: Same procedure used then that described for 1,2-di-dodecanoyl-3-Boc-Lys(boc)-Trp-Lys(boc)-rac-glycerol.

1,2-Di-oleyl-3-Boc-Lys(boc)-Gly-Lys(boc)-rac-glycerol: Same procedure used then that described for 1,2-di-dodecanoyl-3-Boc-Lys(boc)-Trp-Lys(boc)-rac-glycerol.

1,2-Di-tetradecanoyl-3-Lys-Gly-Lys-rac-glycerol: Same procedure used then that described for 1,2-di-dodecanoyl-3-Lys-Trp-Lys-rac-glycerol.

1,2-Di-oleyl-3-Lys-Gly-Lys-rac-glycerol: Same procedure used then that described for 1,2-di-dodecanoyl-3-Lys-Trp-Lys-rac-glycerol.

### Example 10

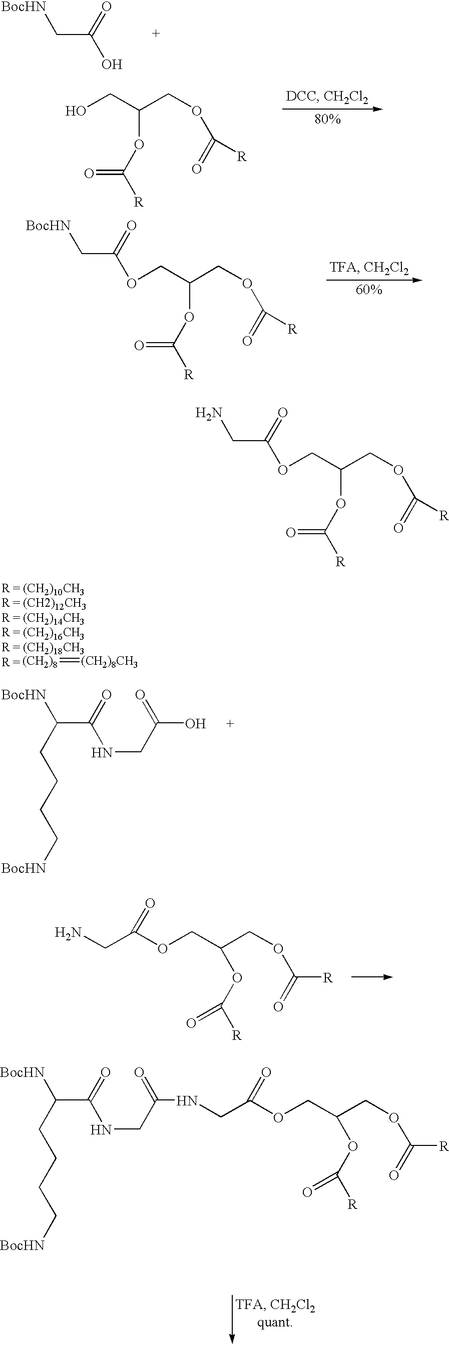

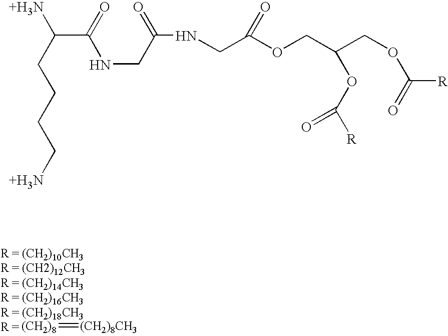

1,2-Di-tetradecanoyl-3-Boc-Gly-rac-glycerol: To solution of Boc-Gly-OH (1 mmol), 1,2-di-tetradecanoyl-rac-glycerol (1 mmol) and DMAP (catalytic amount) in DCM (20 mL) was added DCC (1.1 mmol). After the addition, the solution stirred for 18 h. The reaction mixture was then filtered to remove the insoluble DCU. Concentration of the filtrate followed by chromatography (Hexane/EtOAc 8:2) afforded the product as white powder.

1,2-Di-tetradecanoyl-3-Gly-rac-glycerol: The 1,2-di-tetradecanoyl-3-Boc-Gly-rac-glycerol was dissolved in a mixture 1:9 TFA/dichloromethane. After 2 h stirring, the solvent was removed, the residue washed with ether several and dried under vacuum.

1,2-Di-tetradecanoyl-3-Boc-Lys(boc)-Gly-Gly-rac-glycerol: Same procedure used then that described for 1,2-di-dodecanoyl-3-Boc-Lys(boc)-Trp-Lys(boc)-rac-glycerol.

1,2-Di-tetradecanoyl-3-Lys-Gly-Gly-rac-glycerol: Same procedure used then that described for 1,2-di-dodecanoyl-3-Lys-Trp-Lys-rac-glycerol.

### Example 11

### Preparation of 2,3-Bis-(benzyloxy)-12-(oxododecanoyloxo)-N-(2-hydroxy-ethyl)-N,N-dimethyl-propan-1-ammonium

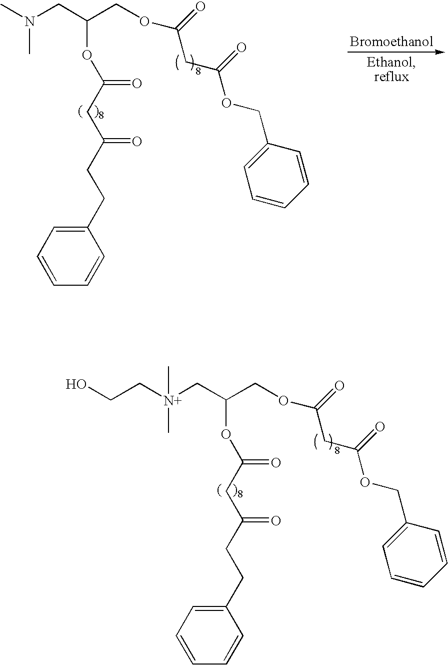

2,3-Bis-(benzyloxy)-12-(oxododecanoyloxo)-N-(2-hydroxy-ethyl)-N,N-dimethyl-propan-1-ammonium: A solution of dodecanedioic acid benzyl ester 2-(11-benzyloxycarbonyl-undecanoyloxy)-3-dimethylamino-propyl ester (1 mmol), bromoethanol in ethanol was reflux for 48 h. The reaction mixture was evaporated and recristalized in ether.

### Example 12

### Preparation of 2,3-Bis(12-(benzyloxy)-12-oxododecanoyloxy)-N,N-bis(2-hydroxyethyl)-N-methylpropan-1-ammonium

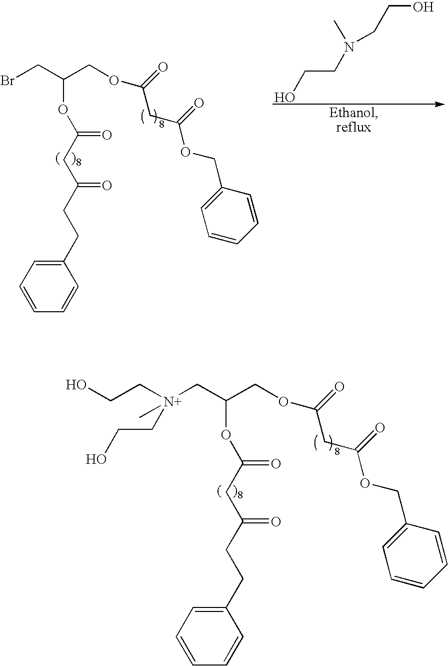

2,3-Bis(12-(benzyloxy)-12-oxododecanoyloxy)-1-bromopropan: To solution of 3-bromoethanol (1 mmol), dodecanoicacid benzyl ester (1 mmol) and DMAP (catalytic amount) in DCM (20 mL) was added DCC (1.1 mmol). After the addition, the solution stirred for 18 h. The reaction mixture was then filtered to remove the insoluble DCU. Concentration of the filtrate followed by chromatography (Hexane/EtOAc 8:2) afforded the product as white powder.

2,3-Bis(12-(benzyloxy)-12-oxododecanoyloxy)-N,N-bis(2-hydroxyethyl)-N-methylpropan-1-ammonium To a solution of Malonic acid benzyl ester 1-benzyloxycarbonylacetoxymethyl-2-bromo-ethyl Ester (1 mmol), diethanol methylamine (1.2 mmol) in ethanol was reflux for 48 h. The reaction mixture was then evaporated, followed by chromatography (AcOEt/Methanol 8:2) afforded the product as yellow powder.

### Example 13

### Preparation of 12-(benzyloxy)-N-(12-benzyloxy)-12-oxododecyl)-N,N-dimethyl-12-oxododecane-1-ammonium

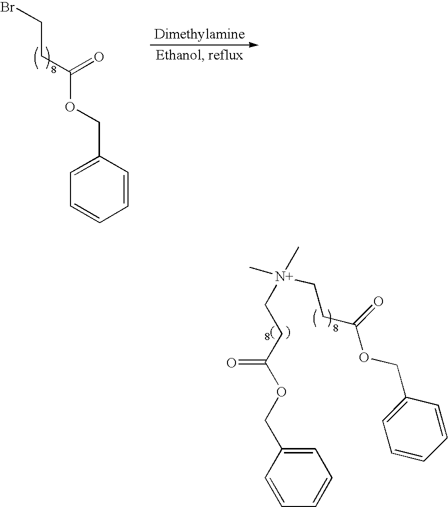

Benzyl 12-bromododecanoate: To solution of 1-bromododecanoic acid (1 mmol), benzyl alcohol (1.1 mmol) and DMAP (catalytic amount) in DCM (20 mL) was added DCC (1.1 mmol). After the addition, the solution stirred for 18 h. The reaction mixture was then filtered to remove the insoluble DCU. Concentration of the filtrate followed by chromatography (Hexane/EtOAc 9:1) afforded the product.

12-(benzyloxy)-N-(12-benzyloxy)-12-oxododecyl)-N,N-dimethyl-12-oxododecane-1-ammonium: To a solution of 3-benzyl 12-bromododecanoate (1 mmol), dimethylamine (1.2 mmol) in ethanol was reflux for 48 h. The reaction mixture was then evaporated, and the residue was recristalized in ethanol to afforded the product as white powder.

### Example 14

Exclusion assay (adapted from; A. J. Geall, I. S. Blagbrough, Journal of Pharmaceutical and Biomedical Analysis 22 (2000) 849-859)

Five μg (5 μl of 1 mg/mL solution) of DNA and varying amount of amphiphiles (dependent on the Amphiphile/DNA ratio required) were diluted to 1000 μL with buffer (2 mM HEPES, 150 mM NaCl, 10 μm EDTA, pH 7.4). The solutions were mixed on a bench top vortex and incubated for 60 minutes at ambient temperature. Each solution was then diluted to 3 mL with buffer (2 mM HEPES, 150 mM NaCl, 10 μm EDTA, pH 7.4). Immediately prior to the analysis, 3 μL of Eth Br solution (0.6 mg/mL, 1.3 mM, effectively present in excess) was added, the sample was mixed on a bench top vortex, and the fluorescence measured. The fluorescence was expressed as the percentage of the maximum fluorescence signal when EthBr was bound to the DNA in the absence of amphiphile. Assays were run in triplicate. Next an esterase was added to the solution which cleaved the ester linkages to afford the anionic compound, releasing the DNA from the amphiphile and enabling the EthBr to intercalate in the DNA. This experimental result demonstrates that a functional synthetic vector can bind and release DNA in the presence of an esterase.

### Example 15

Transfection assays were performed using the well established beta-galactosidase transfection assay. In these experiments the beta-galactosidase gene is transfected into cells. Next, the expressed enzyme then cleaves a chemiluminescent reporter that is detected. The assays are conducted with Chinese hamster ovary (CHO) cells following a standard lipid transfection procedure. The procedure is performed on varying concentrations of lipid and DNA in triplicate in 96 well plates.

### DNA Binding Affinities

Binding studies were carried out by competitive displacement fluorimetric assay with DNA-bound ethidium bromide. This assay involves the addition of aliquots of the compound to a 3 mL solution of EthBr (1.3 μM) and calf thymus DNA (3 μM) in buffer (100 mM NaCl, 100 mM Tris, pH 7.4) with the decrease of fluorescence (λ_{exc}=546 nm, λₑₘ=600 nm; 1 cm path length glass cuvette, slit width 3 nm) recorded after 5 min equilibrium time following each addition.

### Cell Culture and Transfection Experiment

Chinese hamster ovarian cells (CHO, ATCC, Manassas, Va.) were cultured in complete F12K media (ATCC) containing 10% fetal calf serum (Sigma) and 1% penicillin and streptomycin (500 IU/ml and 5000 μg/ml, respectively, Mediatech, Herndon, Va.) at 37° C. in 5% CO₂ with humidity. When the CHO cells reached about 90% confluency, they were split into 48-well plates with a 1:4 ratio using a standard trypsin-based technique. Transfections were performed 24 hours later by modification of previously published methods. Briefly, plasmid DNA coding for a reporter gene, β-galactosidase (β-gal, p Vax-LacZ1, Invitrogen) was first mixed with lipids in potassium phosphate buffer (PBS) at room temperature. Depending on the experimental design, the ratio of DNA and amphiphile, the pH of the buffer used, and incubation time was varied. The mixture was incubated for a minimum 15 min at room temperature before adding to the cells. The amount of DNA used was the same as used in naked DNA control and positive control (commercially available transfection reagents). After incubation at 37° C. and 5% CO₂ for 2 hours, medium containing the mixtures was gently removed and fresh growth medium was added. Transfection efficiencies were assessed 24 h to 48 h post transfection depending on the experimental design. Negative controls were constructed with 1.0 mL of serum-free F12 K medium and naked DNA controls were using 1.0 mL of serum-free F12 K medium with 10.0 μl (1 μg) of reporter gene. Positive controls were performed according to the manufacturer's protocol. Briefly 2.0 μl of Transfast® transfection reagent (1 mg/ml) (Promega, Madison, Wis.) was mixed with 10.0 μl (1 μg) of reporter gene in 1.0 mL of serum free F12 K medium for 15 min at room temperature before transfecting cells. Negative controls where constructed with 1.0 mL of serum-free F12 K medium and naked DNA controls were using 1.0 mL of serum-free F12 K medium with 10.0 μL (1 μg) of reporter gene.

### Reporter Gene Transfection Efficiency Assay

Reporter gene (β-gal) assay was performed with a β-galactosidase enzyme assay system (Promega, Madison, Wis.) following the manufacturer protocol. Briefly cells were first lysed using M-PER buffer (Pierce, Rockford, Ill.) and enzyme activities were determined. A standard curve was constructed for each experiment using dilutions of purified β-gal protein. The β-gal activities from experimental samples were determined by comparison to the standard curve (enzyme activity vs. enzyme concentration). Efficiency of each transfection was calculated as β-gal activity normalized to total protein. The peptide-based amphiphiles such as 1,2-di-tetradecanoyl-3-Lys-Trp-Lys-rac-glycerol show high transfection capability. Likewise, compositions containing 1,2-di-dodecanedioyl benzyl ester-3-phospho ethanolamine-rac-glycerol with DOTAP show high transfection capability.

### Example 16

### Cytotoxicity

Cytotoxicity was assessed using both a formazan-based proliferation assay (CellTiter 96 AQueous One Solution Cell Proliferation Assay kit, Promega) and a total protein-based assay (Pierce). Briefly, CHO cells were seeded onto a multi-well microtiter plate with an appropriate density, depending on the size of the well (e.g., 1×10⁴ cells per well in a 96-well plate). After 48 h, MTS substrate was added to each well and the plate and incubated for 4 h at 37° C. in a humidified, 5% CO₂ incubator. The amount of soluble formazan produced by cellular reduction of the substrates MTS was recorded at 490 nm using a multi-well plate reader. For the total protein-based proliferation assay, cells were lysed at the same time when transfection efficiency was assayed. A 5 μL of lysates were transferred to a separate multi-well plate. Total protein contents were assessed using the Coomassie Blue protein kit (Pierce, Rockford, Ill.) following the manufacturer protocol. Negative and positive controls were non-treated cells and commercial lipids treated cells, respectively. The proliferation results were expressed as percentages of non-treated cells. The peptide-based amphiphiles such as 1,2-di-tetradecanoyl-3-Lys-Trp-Lys-rac-glycerol 1,2-di-dodecanedioyl benzyl ester-3-phospho ethanolamine-rac-gylcerol shown minimal cell cytotoxicity.

### Example 17

### Gene Transfection Efficiency in a Cell Population

Once the CHO cells were transfected with the reporter gene (β-gal) and the 1,2-di-tetradecanoyl-3-Lys-Trp-Lys-rac-glycerol reagent, we visualized the cells using optical microscopy. Importantly, more than 70% of the cells had been transfected as compared to less than 40% when using other transfection reagents.

### Example 18

### SiRNA Delivery

Trypsine adherent cells and dilute in normal growth medium to 1×10⁵ cells per ml. Dilute the 1,2-di-tetradecanoyl-3-Lys-Trp-Lys-rac-glycerol reagent in serum free medium, incubate at room temperature for 10 min. Dilute RNA in serum free medium. Mix the diluted RNA with the transfectant reagent, incubate for 10 min at room temperature and dispense into a culture plate. Depending on the experimental design, the ration of lipid, siRNA, pH, and incubation time was varied. Overlay cell suspension onto the transfection mixture. Incubate at 37° C. and 5% CO₂. Assay for gene knockdown were assessed after 48 h depending on the experimental protocol.

The gene knockdown assay performed was KDalert™ GAPDH Assay (Ambion) following the manufacturer protocol. Briefly, 48 hr after siRNA transfection, aspirate the culture medium from transfected cells. Add 200 μl KDalert Lysis Buffer to each sample well. Incubate at 4° C. for 20 min to lyse the cells. Pipet the cell lysate up and down 4-5 times to homogenize the lysate. Transfer 10 μl of each lysate or GAPDH Enzyme dilution (including the GAPDH Working Stock) to the wells of a clean 96 well plate. Working quickly, add 90 μl of KDalert Master Mix to each sample using a multi-channel pipettor to dispense the KDalert Master Mix quickly. Measure the increase in fluorescence of the samples at room temp. Using the reagent above we saw greater than 80% knockdown of the protein.

### Incorporation by Reference

All of the U.S. patents and U.S. published patent applications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

1. A compound represented by Formula I: 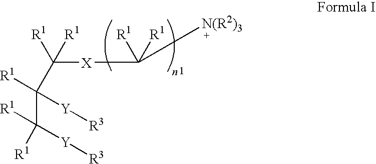
wherein
X represents 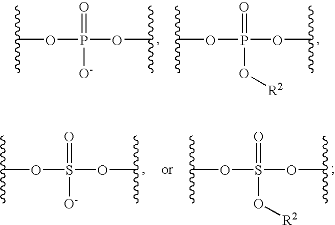
R¹ represents independently for each occurrence H, alkyl, or halogen;
R² represents independently for each occurrence H, alkyl, alkenylalkyl, aryl, or aralkyl;
R³ represents independently for each occurrence alkyl, alkenylalkyl, aryl, aralkyl, 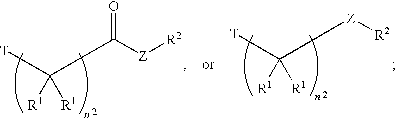
n¹ and n² represent independently for each occurrence an integer from 1-50;
Y and Z represent independently for each occurrence O or —N(R²)—; and
T represents independently for each occurrence —C(R²)₂—, or —C(═O)—.

2-6. (canceled)

7. A compound represented by Formula VII: 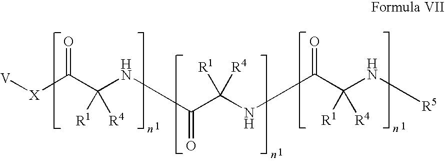
wherein
X represents O, —N(R²)—, —C(═O)—, —C(═O)N(R²)—, —OC(═O)N(R²)—, —N(R²)C(═O)—, or —O—C(═O)—;
V represents 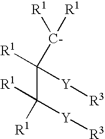
or an optionally substituted saturated or unsaturated cyclopentaphenanthrene ring;
R¹ represents independently for each occurrence H, alkyl, or halogen;
R² represents independently for each occurrence H, alkyl, alkenylalkyl, aryl, or aralkyl;
R³ represents independently for each occurrence alkyl, alkenylalkyl, aryl, aralkyl, 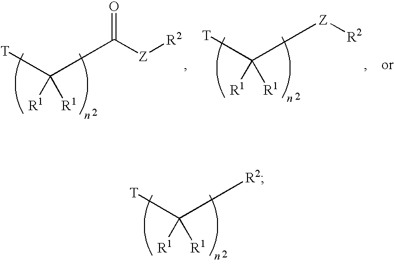
R⁴ represents independently for each occurrence an amino acid side chain;
R⁵ represents independently for each occurrence H, alkyl, alkenylalkyl, aryl, aralkyl, or —C(═O)N(R²)—;
n¹ and n² represent independently for each occurrence an integer from 1-50;
Y and Z represent independently for each occurrence O, —N(R²)—, —O—C(═O)—O—, or O—(C═O)—N(R²)—; and
T represents independently for each occurrence —C(R²)₂—, or —C(═O)—.

8-10. (canceled)

11. A method of delivering a nucleic acid to a cell, comprising the step of contacting a cell with a mixture comprising a nucleic acid; and a compound claim 1 or 7.

12. The method of claim 11, wherein said compound is tethered to a surface.

13. The method of claim 11, wherein said nucleic acid is selected from the group consisting of DNA, RNA, plasmid, siRNA, duplex oligonucleotide, single-strand oligonucleotide, triplex oligonucleotide, PNA, and mRNA.

14. The method of claim 11, wherein said mixture further comprises DPPC, PEGylated DPPC, DMPC, DOPE, DLPC, DSPC, DOPC, DMPE, DPPE, DMPA-Na, DMRPC, DLRPC, DARPC; catonic, anionic, or zwitterionic amphiphile; fatty acid, cholesterol, flourescencetly labeled phospholipid, ether lipid, or sphingolipid; or a combination thereof.

15. The method of claim 11, wherein said cell is a animal cell or plant cell.

16. The method of claim 11, wherein said cell is a mammalian cell.

17. The method of claim 11, wherein said cell is a primate cell.

18. The method of claim 11, wherein said cell is a human cell or insect cell.

19. The method of claim 11, wherein said cell is a human cell.

20. The method of claim 11, wherein said cell is an embryonic cell or stem cell.

21. The method of claim 11, wherein said cell is contacted in vivo.
